(19) United States
(12) Patent Application Publication
(10) Pub. No.: US 2021/0236403 A1
(43) Pub. Date: Aug. 5, 2021

(54) COSMETIC, PERSONAL CARE, AND CLEANING PRODUCTS

(71) Applicant: Jeffrey Dean Lindsay, Appleton, WI (US)

(72) Inventor: Jeffrey Dean Lindsay, Appleton, WI (US)

(73) Assignee: Planet Lindsay, LLC, Appleton, WI (US)

(21) Appl. No.: 17/238,102

(22) Filed: Apr. 22, 2021

Related U.S. Application Data
(63) Continuation-in-part of application No. 17/068,806, filed on Oct. 12, 2020.
Application No. 17/068,806 is a continuation-in-part of application No. 16/926,514, filed on Jul. 10, 2020.
Application No. 16/926,514 is a continuation-in-part of application No. PCT/US2020/055429, filed on Oct. 13, 2020.
(60) Provisional application No. 62/881,212, filed on Jul. 31, 2019.
(60) Provisional application No. 62/872,697, filed on Jul. 10, 2019.
(60) Provisional application No. 62/931,213, filed on Nov. 5, 2019.
(60) Provisional application No. 62/994,810, filed on Mar. 25, 2020.
(60) Provisional application No. 62/914,552, filed on Oct. 13, 2019.
(60) Provisional application No. 63/014,100, filed on Apr. 22, 2020.
(60) Provisional application No. 63/055,305, filed on Jul. 22, 2020.
(60) Provisional application No. 63/066,426, filed on Aug. 17, 2020.
(60) Provisional application No. 63/137,705, filed on Jan. 14, 2021.

(51) Int. Cl. A61K 8/44; A61K 8/67; A61K 8/92; A61K 8/36; A61K 8/34; A61K 8/891; A61K 8/73; A61K 8/24; A61K 8/41; A61K 8/46; A61Q 11/00; A61Q 15/00

(52) U.S. Cl. CPC A61K 8/447; A61K 2800/874; A61K 8/673; A61K 8/922; A61K 8/361; A61K 8/345; A61K 8/891; A61K 8/732; A61K 8/24; A61K 8/416; A61K 8/466; A61Q 11/00; A61Q 15/00; A61K 2800/30; A61K 2800/805; A61K 8/676

(57) ABSTRACT

Products and methods are disclosed relating cosmetic products, personal care products, and cleaning products comprising N-acetyl cysteine. Products for personal care, household use, and other purposes are described with compounds that reduce the odor of N-acetyl cysteine. Skin-care serums with N-acetyl cysteine and Vitamin C are described, as are a variety of other cosmetic and personal care products.

### CLAIM TO PRIORITY

This application claims priority to U.S. patent application Ser. No. 17/068,806, filed Oct. 12, 2020, which claims priority to U.S. Ser. No. 16/926,514, filed Jul. 10, 2020, which in turn claims priority to U.S. Ser. No. 62/881,212, filed Jul. 31, 2019; U.S. Ser. No. 62/872,697, filed Jul. 10, 2019; U.S. Ser. No. 62/994,810, filed Mar. 25, 2020; U.S. patent Appl. Ser. No. 62/931,213, filed Nov. 5, 2019; and U.S. Ser. No. 62/914,552, filed Oct. 13, 2019. This application also claims priority to PCT/US2020/055429, filed Oct. 13, 2020; U.S. Ser. No. 63/014,100, filed Apr. 22, 2020; U.S. Ser. No. 63/055,305, filed Jul. 22, 2020; U.S. Ser. No. 63/066,426, filed Aug. 17, 2020; U.S. Ser. No. 63/137,705, filed Jan. 14, 2021.

### BACKGROUND

### Field of the Invention

This invention pertains to products, formulations, and methods for personal care and cleaning, particularly cosmetics, oral care, and other health care products.

### Description of Related Art

N-acetyl cysteine (hereafter NAC) is a natural compound that has been used, for example, to treat cystic fibrosis and treat overdoses of acetaminophen. It is sometimes used as a nutriceutical. Unfortunately, it suffers from an unpleasant sulfurous odor (and taste), and perhaps for this reason seems to have been largely neglected in the marketplace for personal care and household products. One step in advancing its use was the discovery that biofilms can infect clothing such as athletic wear and that NAC can display synergy with various enzymes in attacking biofilm in clothing, thus providing a useful new tool that can help overcome the problem persistent odor (see U.S. patent application Ser. No. 16/926,514, “Methods and Compositions for Reducing Persistent Odor in Clothing and Mitigating Biofilms,” filed Jul. 10, 2020; and U.S. patent application Ser. No. 17/068,806, “Methods and Compositions for Reducing Persistent Odor in Clothing and Mitigating Biofilms on Various Materials,” filed Oct. 12, 2020 and PCT/US2020/055429, “Methods and Compositions for Reducing Odor and Biofilm,” filed Oct. 13, 2020).

Enhanced control of the sulfurous odor is needed not only for laundering and cleaning with NAC compositions, but also for the rich potential NAC products appear to have in cosmetics and personal care. Such potential may be due, in part, to the antimicrobial and antioxidant properties of NAC, its relationship to biological agents such as glutathione, and its ability to act in synergy with a variety compounds to undermine biofilms, which may play previously underappreciated roles in aspects of skin care, personal care, wound and infection care, household cleaning, etc.

Regarding odor control, many people seek to avoid the embarrassment of body odor associated with perspiration. Rather than relying on fragrance to mask odor or on metal-containing compounds such as aluminum and zirconium salts to plug up sweat pores, alternative approaches have been developed that can reduce odor by adjusting the skin microbiome, the array of microbes that live on the skin. Two patents, U.S. Pat. No. 9,566,223, “Antiperspirants and Deodorants,” issued Feb. 14, 2017, and U.S. Pat. No. 8,992,898, “Antiperspirants and Deodorants,” issued Mar. 31, 2015, describe the use of alpha hydroxy acids, particularly mandelic acid, in combination with caffeine for control of the skin microbiome as a natural deodorant that can dramatically reduce odor without requiring the use of aluminum or zirconium salts, and a third, U.S. Pat. No. 9,668,948, “Products and Methods for Reducing Malodor from the Pudendum,” issued Jun. 6, 2017 describes compositions with mandelic acid for controlling odor from the pudendum. Efforts to use carboxylic acids in significant concentrations have faced difficulty, sometimes because a low pH range can cause instability in the preparation due to incompatibility of many emulsifiers and other agents with low pH (e.g., from about 3 to 4.5 or from 3 to 4 or from 3.2 to 3.9, etc.), or result in skin irritation from a heterogeneous mixture with pockets of elevated acid content that can cause small regions of irritated skin. One challenge is creating a cosmetic product such as a solid deodorant stick at low pH (e.g., 2.5 to 4.3 or 2.5 to 4.1) with low water content, high acid content, and yet have good stability and no evidence of concentrated pockets of acid or crystals of acidic material that can be felt by the skin. In many products, it can be desirable to have a relatively small aqueous phase such as less than 30% by weight of the aqueous phase, or less than 25% water, more specifically less than 20%, 15%, 10%, 9%, 8%, 5%, 4% and 3% water, such as from 1% to 20% water or 1% to 15% water or 1% to 12% water, 1% to 9% water, 1% to 5% water, or 0.5% to 3% water (unless otherwise specified, percentages refer to mass). This may be a desirable goal for reasons such as providing good tactile and aesthetic properties, as well as for product stability, reduced risk of moisture loss, more successful manufacturing due to a smaller water phase in making an emulsion, etc. However, with a small aqueous phase, if one were to see to provide adequate levels of an acidic material such as N-acetyl cysteine or a carboxylic acid such as mandelic acid for odor control, it would seem to require high levels of solids in the heated aqueous phase that could, upon cooling, result in precipitation of the solids and the formation of grit with undesirable tactile properties or pockets of precipitated solids that might be irritating. Fortunately, advances are described herein that yield low pH solids in cosmetic and personal care products that can avert one or more of the problems mentioned above.

Further, new advances are disclosed herein regarding compositions that effectively reduce or substantially eliminate the sulfurous odor of NAC compositions, making such compositions more suitable for consumer use. Further, we have discovered that successful, high-performance acidic deodorant sticks and other products can be made at low pH and with good tactile properties using the approaches described herein. We have also discovered that cosmetic products comprising NAC and related compounds can be produced with excellent properties, such as masks, lotions, creams, and serums for skin care, cleaning, acne control and antimicrobial purposes, etc.

### SUMMARY

For NAC-containing products in fields such a personal care, cleaning, etc., we have discovered that aqueous solutions of NAC, as well as various cosmetic formulations comprising NAC such as emulsions, serums, creams, and solid sticks that normally might have notable sulfurous odor for effective concentrations of NAC can have the odor substantially reduced by adding certain additives such as phosphate or polyphosphate compounds (e.g., phosphate salts, salts of metaphosphate, trimetaphosphate, and/or hexametaphosphate, particularly sodium hexametaphosphate or SHMP), betaine compounds such as coco betaine, sultaine compounds such as hydroxysultaines (e.g., cocoamidopropyl hydroxysultaine), and EDTA. In emulsions, sticks, etc., NAC can be combined with NAC-odor control agents in an aqueous phase that can then be combined with an oil phase or silicone phase (including silicone and oil phases). While the effectiveness of any NAC-odor control agent may depend on pH, etc., we have found sodium hexametaphosphate, for example, to be versatile in its performance under various conditions, often leading other candidates in its odor suppression ability relative to NAC odor.

Thus, in one aspect, a personal care or cleaning preparation is provided comprising at least 0.5% N-acetyl cysteine and an agent effective at reducing the odor of N-acetyl cysteine selected from phosphate salts, polyphosphate compounds, betaine compounds, sultaine compounds, and EDTA. In one aspect. the mass of phosphate salts and polyphosphate compounds is at least 20%, 25%, 30%, 35%, 40%, or 50% of the mass of N-acetyl cysteine. Generally, “personal care compounds” as used here may exclude products used for injection or inhalation by medical professionals, or also those used for ingestion, but rather refer to compounds for topical use on the body or for application to solid surfaces such as in household cleaning of items such as bathroom and kitchen surfaces, fabrics and textiles, or regions where biofilm may be present, including care of facial skin, underarms, the scalp, nails, etc. In a related aspect, a personal care product may comprise 0.5% to 5% N-acetyl cysteine, at least 10% water, an odor control agent effective at reducing the odor of N-acetyl cysteine selected from phosphate salts, polyphosphate compounds, betaine compounds, sultaine compounds, and EDTA, wherein the mass ratio of the odor control agent relative to N-acetyl cysteine is at least 20%.

In a related aspect, a cosmetic or cleaning preparation may comprise at least 0.5% N-acetyl cysteine, at least 1% of a diol or polyol, and at least 0.3% to 6% of an agent effective at reducing the odor of N-acetyl cysteine. The preparation may be in the form of a solid such as a solid stick for application to the body or other surfaces, held in a suitable container, further comprising 10% to 80% of a hydrophobic base comprising lipids, optionally from 5% to 40% silicone materials or a total of at least 40% lipids and silicone materials, and may have less than 20% water such as from 1% to 8% water, with an effective pH of 2.5 to 4, 2 to 4.5, or 2.5 to 3.8, and may contain 1% to 25% starch such as from 4% to 20% starch or at least 4% starch. Alternatively, the solid may comprise than 8% water and from 5% to 40% silicone material and from 15% to 70% lipids. In one aspect, the solid material may comprise an acidic material selected from Vitamin C and derivatives thereof and alpha hydroxy acids, wherein the acidic material plus the N-acetyl cysteine comprises from 3% to 16%, 4% to 23% or 2% to 10% of the preparation, and wherein the preparation comprises at least 40% of a hydrophobic material selected from lipids and silicone materials, and comprises from 0.5% to 10% emulsifiers and from 1% to 25% of a solvent that is liquid at 25° C. selected from water, alcohols, diols, and polyols, wherein the acidic material is soluble in the solvent, such that upon cooling, the solid is substantially free from tangible grit formed from precipitated acidic material, and the preparation is a solid at 25° C. having an effective pH from 2 to 4.5.

In one aspect, a skin-care serum is provided comprising from 0.2% to 6% 0.5% to 5%, or 0.6% to 7% N-acetyl cysteine, from 5% to 20% Vitamin C or derivative thereof, and from 0.2% to 3% panthenol or derivatives thereof, having a pH from 2.4 to 4.3, 2 to 4.5, 2.5 to 4.5, 2.5 to 4.2, or 2.5 to 3.8, optionally being substantially free of acrylamide compounds and having less than 15% lipids.

In one aspect, the preparation may comprise antimicrobial agents such as the cationic steroidal antimicrobial (CSA) compounds described by Paul Savage and D. Leung in U.S. Pat. No. 7,754,705, “Cationic steroid antimicrobial compositions and methods of use,” issued Jul. 13, 2010; Carl Genberg and Paul Savage, U.S. Pat. No. 9,603,859, “Methods and products for increasing the rate of healing of tissue wounds,” issued Mar. 28, 2017; and Carl Genberg, C. S. Beus, and Paul B. Savage, United States Patent Application 20150374719, “Methods for Treating Fungal Infections,” issued Dec. 31, 2015; all of which are hereby incorporated by reference. Any of the compounds described therein such as CSA-13, CSA-25, CSA-54, CSA-90, CSA-92, CSA-190, CSA 191, and CSA 1921 and the like may be combined with the compositions described herein. For example, CSA compounds may be present in an aqueous solution at a concentration of from 0.01% to 1%, such as from 0.01% to 0.5%, or from 0.02% to 0.4% by weight. Such compounds are commercially available from Purishield Life Sciences, LLC (Walnut Creek, Calif.) under the Purishield®, Purifect® or Ceragyn® brands, typically provided at a concentration of 0.4% CSAs.

Cleaning Products with NAC. A related discovery is that NAC can be combined with enzymes and/or surfactants to weaken biofilm in a variety of settings. Without wishing to be bound by theory, it appears that NAC acts as if it softens or loosens the extracellular matrix of many biofilms, thereby increasing the ability of other agents such as enzymes, surfactants, or antimicrobial agents to attack the biofilm and its bacteria. Cleaning benefits have been seen in solutions of NAC and enzymes applied to sinks, showers, toilets, etc. A useful aqueous NAC solution may comprise from 0.3% to 25% NAC, such as from 0.5% to 10%, from 0.5% to 6%, or from 0.5% to 4% such as from 0.5% to 2.5%, with 0.5% to 15% or 1% to 8% enzymes such as protease, lipase, cellulase, pectinase, etc., optionally combined with 1% to 20% surfactants (e.g., anionic, cationic, nonionic), optionally 0.5% to 10% salts such as sodium citrate or lactate, optionally 0.5% to 3% panthenol or derivatives thereof, at a pH from 2 to 11, such as from 3 to 9, 4 to 9, 4 to 7, etc. Such cleaning products may be applied to a surface with biofilm by spray, wipe, sponge application, pouring, etc., and once the surface is wetted, may be allowed to sit for at least 1 minute such as from 3 to 30 minutes or longer if desired, followed by wiping or scrubbing to clear away biofilm material. A melamine foam cleaning product may be used. The surface can then be rinsed and, if desired, treated with other agents such as bleach or antimicrobials.

In some aspects, NAC is combined with other antimicrobial agents such as from 0.02% or 0.1% to 1.5% or 3% dihydro-resveratrol, which may offer unusual synergy in terms of also supplementing the antioxidant function of NAC. Other antimicrobial agents may include at least 0.02% cationic steroidal antimicrobial agents, etc. Likewise, the fermentation products of Tricholoma matsutake and Hericium erinaceum, high in L-ergothioneine, or other compounds also comprising ergothioneine may be considered. Other sources include Mycobacterium smegmatis and certain fungi, such as Neurospora crassa, as well as various beans, etc. without Wishing to be bound by theory, it is believed that ergothioneine, being an amino acid with a thiol group, may function synergistically with NAC for its cosmetic and biofilm benefits. Other “biofilm softening” agents and/or skin softening or skin permeability enhancing agents may be added, including panthenol, for increased efficacy such as, potential increased weakening of biofilm in pores of the skin or other surfaces.

The NAC solution may be applied to surfaces suspected to have biofilm material or odor problems or bacterial infection, and may be allowed to sit where applied for an effective time such as from 1 minute to 24 hours, or from 2 minutes to 8 hours, at least two minutes, at least 10 minutes, at least 20 minutes, or from 10 minutes to 8 hours (unless otherwise specified, these times may overlap with the time other agents are also present that may be added before or after application of the NAC solution, such that a 30 minute dwell time may be achieved by spraying or pouring NAC solution onto an article, and then 5 minutes later also applying an enzymatic mixture, and then allowing the article to sit for another 25 minutes before laundering or washing or further diluting the applied NAC solution and enzymatic mixture, but in some aspects, these times can refer to the time the NAC remains substantially undiluted after application).

An enzyme and/or surfactant solution (a cleaning solution) may further be applied to the region treated with NAC solution, either in a pretreatment prior to laundering or washing by adding a mix that may reside on the treated region for an effective period of time similar to the effective times mentioned for NAC solution or by directly cleaning or laundering with cleaning agents such as laundry detergents comprising enzymes and/or surfactants, wherein the cleaning agents may be directly applied to the regions treated with or to be treated with NAC solution and may be allowed to reside for a period of time (e.g., at least 5 or 10 minutes, 5 minutes to 2 hours, 2 minutes to 30 minutes, etc.) before removal or dilution with the added water of the laundering process. In such two-step operations, the NAC solution may be added first followed by a cleaning solution comprising at least one of enzymes, surfactants, and bacterial spores, or the NAC solution may be added after treatment with the cleaning solution, or the two may be applied at substantially the same time. In such two-step processes, the time between application of the first and second solutions may be less than one minute such as from 5 seconds to 1 minute, one minute or more, two minutes or more, from 2 to 30 minutes, etc. The pH of the NAC solution may be adjusted such that it does not hinder the efficacy of enzymes or bacterial spores. For many laundry detergents, a NAC solution pH may be from 4 to 10, such as from 5 to 9, 5.5 to 10, and 6 to 9. The cleaning solution may optionally comprise bacterial spores such as Subtilis bacillus spores and other spores.

While NAC can interfere with enzyme activity in several ways, we have found that by properly adjusting concentration and pH, NAC can be combined with laundering enzymes with excellent results, including enhanced stability of enzymes over time. For example, a NAC solution can be prepared with from 0.2% to 6% NAC in a solution having from 0.5% to 10% or from 1% to 5% enzymes (solids basis) such as a mixture of lipase, protease, cellulase, mannanase, and amylase such as mixtures marketed for laundry detergents by Novozymes, 5 to 20% surfactant, 1 to 10% salts, 40% or more water, etc., and optional bacterial spores. Bacterial spores tend to be most effective when given at least 2 hours of dwell time in the presence of foodstuffs that may be present on the article being treated, and can produce a variety of enzymes that can attack foodstuffs, including biofilm in the presence of NAC.

A related discovery is that UV light can be used to visualize the presence of biofilm in clothing, particularly for synthetic fibers. The use of optical whiteners in laundry detergents or the direct application of solutions of such whiteners (e.g., Calcafluor White) can result in optical whiteners preferentially attaching to biofilm material, allowing fluorescence in UV light to reveal where biofilm has accumulated. This can guide detection of biofilm for more efficient selection of regions to treat with the products and methods described herein.

We have also found that cosmetic formulations high in NAC can be made in the form of solid or semi-solid sticks for use in odor control or other objectives, even at low pH, a condition which defeats many prior approaches to making deodorant sticks. We have also found that useful creams, lotions, pastes, solutions, oral care products, etc. can be manufactured with NAC that may be of use in various topical and personal care applications. These advances are detailed more fully in US patent application Ser. Nos. 63/014,100, filed Apr. 22, 2020; 63/055,305, filed Jul. 22, 2020; and 63/066,426, filed Aug. 17, 2020. NAC-related products for biofilm control are detailed in U.S. patent application Ser. No. 16/926,514, “Methods and Compositions for Reducing Persistent Odor in Clothing and Mitigating Biofilms,” filed Jul. 10, 2020, and U.S. patent application Ser. No. 17/068,806, “Methods and Compositions for Reducing Persistent Odor in Clothing and Mitigating Biofilms on Various Materials,” filed Oct. 12, 2020.

Applicant has found that successful acidic antiperspirant sticks can be made in compositions comprising lipids or lipids and silicone compounds by combining an oil, silicone, or oil-silicone phase with a relatively viscous acidic phase comprising one or more acidic components such as mandelic acid or other alpha-hydroxy acids and N-acetyl cysteine, wherein a the viscous acidic phase has a viscosity substantially greater than that of water, such as at least 5 times higher (e.g., at least 5 centiStokes or cst). The acidic thickener such as an aqueous starch mixture, a polyol such as propane diol, gums or water-swellable polymers or minerals dispersed in water, etc., is present in the aqueous phase, making it substantially more viscous than water. Such a thickened acidic aqueous phase can be combined with a heated oil phase or oil-silicone phase in the presence of an emulsifying-effective amount of a dermatologically-acceptable emulsifier and/or a gelling-effective amount of a dermatologically-acceptable gelling agent, after which optional agents such as dry powders (e.g., starch, silica materials, silicone powders such as polymethylsilsequioxane, etc.) can be combined with the emulsion or blend of an aqueous phase and an oil or oil-silicone phase, along with other finishing agents such as preservatives, fragrances, other silicone liquids, volatile materials such as volatile silicones, etc. In general, all ingredients should be safe for the quantities used for products intended for use on human skin. Some compositions described herein may also be useful in a variety of other contexts such as for pet care, for odor control on textiles or other surfaces, for cleaning, for reducing microbial growth such as the growth of fungus, mildew, mold, yeast, bacteria, viruses, etc., and thus may have effective amounts of some ingredients that might not be ideal for use on human skin, and should then be provided with suitable instructions and/or warnings to guide users to apply the product properly or to understand what applications are improper.

In some aspects, the acidic stick has from 10% to 50% lipids, from 10% to 40% silicones, from 5% to 25% starches or starch derivatives, from 0.5% to 10% acidic agents such as mandelic acid and N-acetyl cysteine (NAC), and may have less than 20%, 15%, or 10% water. The stick may be formed by providing the acidic components in a thickened aqueous phase having a viscosity of at least 5 cps at 25° C. which is heated and blended into an oil phase or oil-silicone phase or silicone phase, followed by blending other components, such as powders or finishing agents.

Thus, in one aspect, a novel acidic stick comprise a solid or semi-solid waxy phase comprising one or more waxes and/or other lipids, a starch or starch derivative, a thickener, and at least 1% of an alpha-hydroxy acid (e.g., mandelic acid, citric acid, glycolic acid, lactic acid, malic acid, tartaric acid, etc.) associated with the thickener, wherein the alpha-hydroxy acid is substantially uniformly dispersed in the stick. The acidic stick may have from 0.2% to 20% water (e.g., from 3% to 15%), 1% to 9% alpha hydroxy acid, 5% to 35% silicone compounds, 10% to 40% lipids, 0.5% to 10% emulsifiers or gelling agents, and 2% to 20% starch or starch derivatives. It may also comprise from 0.5% to 10% of a polyol and optional antiperspirants.

We have found that successful acidic deodorant sticks can be made in relatively low-moisture formulas with silicones and lipids or substantially anhydrous formulations, achieving high levels of acidic ingredients such as mandelic acid and/or N-acetyl cysteine, without creating compositions that can are perceived as gritty. In particular, we have found that certain techniques such as significantly elevating the viscosity of the aqueous phase, in combination with carefully selected ingredients and other innovative formulation methods, can overcome multiple problems that hindered the development of a successful deodorant stick with high mandelic acid content. Through the approaches and formulations described herein, we have found that high levels of mandelic acid and other solids can be present in a minor aqueous/polar phase that, when combined with an oil/non-polar phase (including silicone or oil-silicone phases), can subsequently be cooled to room temperature without leading to the formation of perceptible grit and without evidence of skin irritation from nonuniformity in the distribution of acidic components. In some aspects, there may be no perceptible indication of alpha hydroxy acid precipitation at all, and if particles do precipitate, they may be so fine as to provide virtually no tactile clue of their existence. The resulting solid can have a smooth feel that can be applied comfortably and can be used with successful odor control without undue risk of skin irritation.

Surprisingly the approaches described herein seem to be able to solve multiple problems at once, including one or more of: a) difficulties in forming a stable dispersion involving oil and water phases at low pH, b) the problem of skin irritation from large pockets of alpha hydroxy acid in the cooled stick, c) the problem of poor texture due to a gritty feed from precipitated solids that were once in or largely in the aqueous phase, and d) the difficulty of having the acidic components sufficiently accessible to the skin to be able to modify the skin microbiome and/or effectively reduce malodor from certain bacteria. Note, however, that the ability of some aspects to overcome more than one obstacle or to provide more than one benefit should not be taken as a requirement that all aspects as claimed must necessarily solve the same plurality of problems of provide the same plurality of benefits.

Further, we have discovered that acidic sticks, creams, masks, and serums can be made acidic not only with alpha-carboxylic acids such as mandelic acid or lactic acid or other acids such as Vitamin C, but may have other positive effects on the skin and skin microbiome using N-acetyl cysteine, which may help hinder growth of biofilms and undesirable bacteria while also having other skin health benefits.

Surprisingly, we have found that by significantly elevating the viscosity of the aqueous phase, in combination with carefully selected ingredients and innovative formulation methods, we can overcome multiple problems that hindered the development of a successful deodorant stick at low pH. Surprisingly, through the approaches and formulations described herein, we have found that high levels of mandelic acid and other solids can be present in a minor aqueous/polar phase that, when combined with an oil/non-polar phase (including silicone or oil-silicone phases), can subsequently cool without forming perceptible grit and without evidence of skin irritation from nonuniformity in the distribution of the acidic components. The resulting solid can have a luscious, smooth feel that can be applied comfortably and can be used with successful odor control without undue risk of skin irritation.

We have therefore found that personal care deodorant and antiperspirant compositions comprising effective levels of alpha hydroxy acids such as mandelic acid and/or other acidic materials can be formulated in a solid stick for convenient application to the underarms or other regions of the body. Such sticks can also comprise caffeine and may be substantially aluminum free and zirconium free or may comprise significant amount of aluminum and/or zirconium compounds, such as at least 3% by weight, at least 5%, 8%, 10%, 12%, or 13%, such as from 3% to 30%, 3% to 25%, 3% to 20%, 5% to 24%, etc. In some aspects, the composition is prepared by combining one or more acidic materials such as mandelic acid in a solvent to create an “acid paste” having a viscosity substantially greater than water such as at least 5, 50, 100, or 200 times greater than water. The acid paste may comprise water, water and one or more polar solvents, or a polar organic solvent other than water, with an thickener such as a starch, a gum, minerals such as laponite, polymers such as polyacrylate and other acrylate polymers or copolymers (e.g., acrylates/C10-30 alkyl acrylate crosspolymer, crosslinked copolymers, such as Carbopol® Aqua SF-1 or Polyacrylate-14 marketed by Lubrizol Corp.) or carbomer (crosslinked homopolymers of acrylic acid, e.g., Carbomer 980) or polyquaternium compounds (e.g., Polyquaternium 4, 7, 11, 47, or any of the compounds discussed in “Polyquaternium,” Wikipedia, https://en.wikipedia.org/wiki/Polyquaternium), high-viscosity polar solvents, gelatin, or other agents that swell in water or other solvents. (In some aspects, though, it or other products herein may be substantially free or jave less than 2%, 1%, 0.5, or 0.2% of polyquaternium compounds, or acrylates, acryla-mides, or crosslinked polymers.) The resulting viscous acidic mixture is, as defined herein, an “acid paste” that can be combined with a non-aqueous phase (an oil and/or a silicone phase) to form an emulsion or other mixture that can be cooled to form a solid stick, optionally after adding ingredients such as fragrances, powders (e.g., starch, silica, silicone materials, other solids), liquids (e.g., esters, alcohols, or silicone liquids such as an alkyl silicone liquid), etc. The acid paste when added to the other ingredients, prior to evaporation of water, may comprise 1% to 35% of the mixture, such as from 3% to 25%, 3% to 20%, 3% to 15%, and 5% to 17%.

For example, we have found that an aqueous solution of mandelic acid or other soluble acidic materials can be used, such as a solution comprising from 2% to 40% total of mandelic acid and/or NAC in water at a suitable temperature, or water combined with other polar solvents such as propanediol, glycerin, ethanol, propylene carbonate, or other alcohols, glycols, or esters, or in some aspects, in a solution of such polar organic solvents that may be substantially free of water. In some cases, the solution may be supersaturated or substantially saturated.

The viscosity of the acid solution can be elevated to be substantially greater than that of water using a thickener such as a gum, a starch (corn starch, tapioca starch, potato starch, cassava, arrowroot starch, chemically modified starches such as modified food starch, cold-water soluble starches, and the like), a polymer such as a polyacrylate or copolymers thereof or known superabsorbent polymers, hydroxymethyl cellulose or other water soluble cellulosic derivatives, water-swellable polyurethanes such as those described in WO2004029125A1, polyethylene glycol (either liquids such as PEG 400 or aqueous solutions of solids such as polyethylene glycol 3350) and other polymeric polyols, etc. Thickener levels relative to the solvent mass may range from, for example, 0.1 to 15 weight percent, such as at least 0.3, 0.5, 1, 2, 3, 4, 5, or 6 weight percent, up to one of any suitable integer from 2 to 15 weight percent, from 2 to 10 wt %, from 2 to 6 wt %, etc. When starch is used, the starch and solvent is then heated until the starch grains swell (gelatinize) and cause the slurry to become thickened. In water-starch slurries, this may occur between about 50° C. and 80° C., for example, with many native starches tending to gel around 60° C. to 71° C. Rather than gelling with the addition of heat, a soluble starch may be used that is soluble in cold water. The resulting acidic starch paste has elevated viscosity and reduced opacity relative to the initial slurry. An appropriate amount of this slurry, which may be heated to temperature from 40° C. or 50° C. to 70° C. or 80° C., for example, and can then be combined with a molten waxy phase or oil-silicone phase to create a dispersion or emulsion that does not readily separate. Emulsifying waxes, other emulsifiers, or gel-producing agents such as those comprising hectorite particles or other minerals may be present but need not be used in some aspects.

The dispersion may be continually stirred or otherwise blended using rotary mixers, whisks, homogenizers, static mixers, etc., and if desired may be kept at an elevated temperature for a suitable time to promote evaporation of some of the solvent or especially a portion of the water if desired. The dispersion may then be blended with additional agents such as powdered starch or other powders including laponite, talc, hydroxyapatite and derivatives thereof, magnesium hydroxide, magnesium stearate, zinc stearate, zinc oxide, other zinc compounds, antiperspirant salts such as aluminum and zirconium salts, silica, sillylated silica or other solids, microspheres, and the like. In some aspects, however, the deodorant stick is substantially free of aluminum salts and/or substantially free of zirconium salts.

In another aspect, a water/oil emulsion or dispersion comprising mandelic acid associated with an aqueous solvent in an aqueous phase is heated to drive off a portion of the water such as at least 20%, at least 40%, or at least 60% of the water, with exemplary ranges of 20% to 90% or 30% to 80%, resulting in a highly uniform distribution of mandelic acid throughout a waxy phase. Continued stirring may be applied during heating as water is driven off. The peak temperature of the mixture in this process may be at least 75° C., 80° C., 90° C., 95° C., 99° C., 100° C., 110° C., 115°, or in general any whole number between 75° C. and 150° C., such as from 85° C. to 140° C. or 85° C. to 130° C. or from 90° C. to 125° C. The composition may remain above 75° C., 80° C., 85° C., 90° C., 95° C., 99° C., 100° C. or 110° C. for at least 1 minute or any whole number of minutes from 1 to 90, or for at least 2 minutes.

For example, mandelic acid may be dissolved in a mixture of water and an alcohol such as ethanol that may also comprise other soluble material or other solvents such as glycerin or propanediol, and this ethanol-water-acid mixture is combined with a mix of waxes, fatty acids, esters, butter, oils, and related lipids, optionally including at least one emulsifying wax or gel-producing agent. If no thickening agent is included, emulsifying waxes can be particularly helpful in promoting good mixing of the aqueous phase with the oil phase. As the combination is heated and stirred, the solids melt and begin to form a dispersion or emulsion with the ethanol-water-acid mix dispersed in the oil phase. As heating continues, water and ethanol may be progressively driven off, resulting in a waxy material with mandelic acid finely dispersed throughout. Prior to cooling, the melt may be combined with a starch such as arrowroot or tapioca starch to provide additional body and tactile properties, and may then be poured into a suitable container.

Thus, in one aspect, we have developed a personal care composition in the form of a deodorant stick for reducing at least one of perspiration and body odor, the stick comprising from 0.2% to 12% by weight of acidic materials particularly solids (when dry) distributed substantially uniformly throughout a solid or semi-solid waxy phase, such that the composition at room temperature is free of tangible or visible acid grains or crystals in a suitable carrier for application to the skin. In some aspects, the acid such as mandelic acid or NAC is associated with starch granules dispersed throughout the stick that may be swollen, never swollen, or swollen and at least partially dried after contact with the mandelic acid. In some aspects, the stick comprises two forms of starch, one that was previously gelatinized (swollen) in the presence of mandelic acid, and one that was added as unswollen particles to a molten waxy mix comprising previously the swollen starch particles associated with mandelic acid. Both the swollen and unswollen starch particles may independently comprise one or more starches such as corn starch, tapioca starch, pea starch, potato starch, cassava starch, and arrowroot starch.

In some aspects, silicone materials such as cyclopentasiloxane, dimethicone, silica silylate, silica dimethyl silylate, trimethylsiloxysilicate and trifluoropropyldimethyl/Trimethylsiloxysilicate and other siloxanes can be present and may be combined with waxes and oils to impart slip or other tactile or rheological properties or to improve delivery when rubbed against the skin. The silicone content may be from 1 to 50%, such as from 1% to 40%, 5% to 40%, 3% to 36%, 1% to 25%, 8% to 39%, or from 11% to 40%. Silicone-treated silica, silicone-coated particles, silicone microspheres, and other particles comprising silicone may be considered.

In some aspects, the composition may be substantially free of volatile silicone compounds such as volatile cyclic silicone oils, in particular cyclotrisiloxane, cyclotetrasiloxane, cyclopentasiloxane, and cyclohexasiloxane. Other volatile silicone oils that may be substantially excluded include linear or branched silicone oils including 2 to 10 siloxane units, such as hexamethyldisiloxane, octamethylt-risiloxane, and decamethyltetrasiloxane. In some aspects, the composition may be substantially free of volatile silicone oils or any silicone oils having a viscosity at 25° C. of 5 cst or less. The composition may also be substantially free of volatile nonsilicone oils, such as any one or more of isodecane, isoundecane, isododecane, isotridecane, isotetradecane, isopentadecane, and isohexadecane, or may be substantially free of C8-C20 isoparaffins or C8-C16 isoparaffins. The effective pH may be from 2 to 6, such as from 2.8 to 5.7, from 3 to 5.5, from 3.2 to 5.5, and from 3.5 to 5.4.

Without wishing to be bound by theory, one role of a low pH of the skin is to limit the growth of the bacteria that produce undesirable odors. Such bacteria can include Corynebacteria and Propionobacteria. A reduced pH may also create an environment that protects or maintains healthy microbial flora on the skin, thereby controlling the levels of less desirable bacteria that may produce unwanted odor.

We have also found that a variety of creams, pastes, lotions, and other cosmetic or personal care products can be made using NAC and other related compounds such as N-acetyl glucosamine, N-acetyl methionine, L-cysteine, and nacystelyn (NAL), a lysine salt of N-acetylcysteine (NAC) and salts of any of the above such as, for example, L-cysteine HCL monohydrate. For example, NAC or related compounds dissolved in a solvent such as propane diol, water, water and a thickener, propylene glycol, or other solvents and combinations thereof can be blended with a variety of known lotion products or formulations, optionally with additional agents such a sodium methyl cocoyl taurate, GLDA, EDTA, hydroxysultaine or betaine compounds, or various compounds that are effective in reducing the odor of NAC and/or the flavor of NAC, which can hinder consumer acceptance of many cosmetic and oral care products if NAC is added at a significant level (e.g., above 0.1%, 0.3%, 0.5%, 1% etc., such as from 0.1% to 10%, or any numerically feasible combination of lower and upper limits each selected from the series of numbers between 0.2% and 15% in increments of 0.2%, i.e, 0.2, 0.4, 0.6, . . . 14.8, 15.0, such as from 0.4% to 14.2% or 0.2% to 12%). In preparing a lotion, cream, paste, or other cosmetic or personal care product, the NAC or related compound is generally dissolved in an aqueous phase or hydrophilic phase that may comprise anhydrous solvents, which may comprise gelatinized starch, thickening polymers, gelatin, gums, high-viscosity polyols and other thickeners, and may then be blended in with an oil phase or a cream, lotion, paste, ointment, or other cosmetic or personal care agent. Odor control agents may be provided that interact with NAC to reduce its characteristic odor, or flavor control agents such as combinations of essential oils in sorbitol or other sugar alcohols, etc., may be added.

### Definitions

As used herein, a personal care product includes cosmetic products such as skin care creams, lotions and serums and other beauty and skin health aids; hair care products such as shampoos and scalp serums; sunscreens; lip balms; deodorants, antiperspirants, soaps, bodywashes, and agents to reduce or improve body odor and cleanliness; topical agents for controlling inflammation, acne, eczema, and other skin conditions; compounds for enhancing the appearance or health of nails including agents for reducing fungal infections of nails; oral care products such as mouthwash, toothpaste, dental floss, and the like; etc.

As used herein, “deodorant” refers to compositions that are commonly used to reduce unwanted body odors associated with perspiration and/or bacteria on the surface of the skin. “Deodorants” may reduce odor through a variety of means, and such means in the various embodiments of the present invention may include suppression of bacterial activity, antimicrobial mechanisms, chemical interference with odor generation mechanisms, removal or modification of feedstuff for odor-producing bacteria, masking of odors, absorption of odorous materials, and the like.

As used herein, “antiperspirant” refers to materials that help reduce perspiration on the skin, and are often also relied on to reduce odor that may be generated by bacteria acting upon sweat. Many common antiperspirant agents act by forming plugs that block the pores associated with sweat glands or otherwise reducing the flow of sweat. A deodorant may function as an antiperspirant but need not do so to be a deodorant. Antiperspirants recognized for use in the United States include aluminum chlorohydrate, aluminum chloride, aluminum zirconium trichlorohydrate, aluminum zirconium trichlorohydrex gly (a mixture of monomeric and polymeric Zr4+ and Al3+ complexes with hydroxide, chloride and glycine), aluminum zirconium tetrachlorohydrate, aluminum zirconium tetrachlorohydrex gly, aluminum chlorohydrex polyethylene glycol, aluminum chlorohydrex propylene glycol, aluminum dichlorohydrate, aluminum dichlorohydrex polyethylene glycol, aluminum sesquichlorohydrate, aluminum sesquichlorohydrex polyethylene glycol aluminum sesquichlorohydrex propylene glycol aluminum zirconium octachlorohydrate, aluminum zirconium octachlorohydrex gly, aluminum zirconium pentachlorohydrate, aluminum zirconium pentachlorohydrex gly, aluminum zirconium tetrachlorohydrate, and aluminum zirconium tetrachlorohydrex gly, compounds which typically are allowed at levels up to 20 or 25 weight percent.

As used herein, “derivatives of panthenol” may include pantothenic acid and salts thereof (e.g., the calcium, sodium, potassium salts, etc.), pantethine, pantetheine, salt (e.g, sodium or calcium salts) of pantetheine-S-sulfonate, and so forth. Panthenol is closely related to its derivative, pantothenic acid, and pantethine (bis-pantethine or co-enzyme pantethine), a dimeric form of pantetheine produced from pantothenic acid (vitamin B5) by addition of cysteamine. Most vitamin B5 supplements are in the form of calcium pantothenate. However, in one aspect, a composition may be substantially free of pantothenic acid while containing panthenol or derivatives thereof. Without wishing to be bound by theory, panthenol's efficacy against perma-odor and biofilms in infected fabrics may relate to the uptake of panthenol by microbes that need pantothenic acid, wherein the similarity to panthenol “fools” microbes into taking up panthenol as if it were a nutrient when it is not. Such a possibility in another context is proposed in G. F. Helaly et al., “Dexpanthenol and propolis extract in combination with local antibiotics for treatment of Staphylococcal and Pseudomonal wound infections,” Archives of Clinical Microbiology 2/4 (December 2010). If that mechanism is applicable Applicant's results, then panthenol or its derivatives substantially free of panthothenic acid may be especially useful. However, it may also be that panthenol (a.k.a. dexpanthenol) has a softening effect or other secondary effect on the biofilm of an infected fabric.

As used herein, “effective pH” refers to a measure of the pH of a solid or semi-solid deodorant material or related material when it is combined with distilled water. About 0.100 g (e.g., from 0.085 to 0.13 g) of the material is placed in a weighing dish and combined with a mass of distilled water equal to twice the mass of the matter being tested. The weighing dish should be the 7-ml volume plastic intermediate dish provided with the Smart Weight Gem50 jewelers balance (0-50 g, milligram precision digital scale). The material being measured is combined with water and then smeared in the dish by hand to contact the water thoroughly with the material, blending for about 10 seconds. After 15 more seconds, a pH paper strip is contact with the water phase to read the pH. The pH paper may be the Hydrion® 3.0 to 5.5 strip, the Hydrion® 0 to 6.0 strip, or the Lab Essentials™ Universal 1-14 pH Paper, relying on the paper with the smallest range that encompasses the pH of the material being measured. Note that with pH paper, as water wicks up the paper, its pH may change as acidic components are absorbed by or reacted with components in the paper, so the leading front of water wicking into the paper may display a more neutral pH than what is indicate in the main body of the wetted paper, so the pH near the leading edge of the wicking front should be disregarded. In some aspects, the deodorant stick materials disclosed herein may have effective pH values less than 7, such as from 2 to 6.5 or any other pH range discussed herein or with upper or lower limits selected from any pH value discussed herein or in other citations.

As used herein, a “solvent” for dissolving mandelic acid, caffeine, N-acetyl cysteine, or other solids includes water, aqueous solutions, or a variety of other suitable compounds alone or in combination with water or other solvents described herein, such as propylene carbonate, ethanol, propanol, butanol, 1-3 propanediol, 1-2 propanediol (also known as propylene glycol), 3-phenyl-1-propanol, (2,2-Dimethyl-1,3-dioxolan-4-yl)methanol (also known as Solketal, isopropylidene glycerol, or Augeo Multi Clean), glycerin, pentylene glycol, 2-methoxy-2-phenylethanol, and 2-phenylethanol. Such polar organic solvents may also be used without added water to create a low-water or substantially water-free acid paste. In some cases, a solvent can also be suitable as a thickener or part of a thickener composition.

As used herein, “acid paste” refers to an aqueous or polar/hydrophilic phase comprising mandelic acid that is combined with a non-aqueous/non-hydrophilic phase (e.g., an oil phase or silicone phase) in the methods for preparing an acidic deodorant stick disclosed herein. The “acid paste” is a relatively viscous aqueous solution or gel of an acid such as a carboxylic acid, particularly mandelic acid, and generally comprises water, at least one carboxylic acid such as mandelic acid, one or more thickeners and/or relatively viscous polar organic solvents, and optionally a base or buffering agents (e.g., salts such as sodium hydroxide or potassium hydroxide, bicarbonates, carbonates such as sodium carbonate, and the like) to bring the effective pH of the final product to a desired level. The acid paste may comprise caffeine or other agents that are more readily dissolved or dispersed in an aqueous or polar/hydrophilic phase than in an oil or oil-silicone phase.

While a dictionary definition of “paste” can be “a thick, wet substance used for sticking things together, or any soft, wet mixture of powder and liquid” (https://dictionary.cambridge.org/us/dictionary/english/paste), the term “paste” as used in the term “acid paste” (not necessarily for toothpaste or other known consumer products that are called “pastes”) was initially selected based on the successful use of pastes made with starch powder and water combined with mandelic acid and cooked to gelatinize or at least partially gelatinize the starch to form a viscous paste. Gelatinized starch such as arrowroot starch, corn starch, pea starch, etc., in water is typically described as a paste and is a suspension of swollen starch vesicles suspended in water, not actually a solution nor a colloid, though often mislabeled as such. However, the term “acid paste” as used herein can more generally to other thickened aqueous or hydrophilic preparations that are substantially more viscous than water, including aqueous preparations made with starches, flours, gums, superabsorbent or swellable polymers such as polyacrylates or acrylic cross polymers or cellulose derivatives, swellable minerals such as laponite, viscous fluids such as glycerin or propylene glycol, and the like.

Acid pastes are generally aqueous but need not have substantial water content. In some aspects, other hydrophilic solvents can be used. For example, we have demonstrated that 1-3 propane diol, glycerin, and propylene glycol can all be used to dissolve both mandelic acid and caffeine at useful concentrations for preparing a deodorant stick with useful levels of mandelic acid and optionally caffeine. For example, we have demonstrated that 5 ml of these compounds can successfully dissolve 0.5 g of mandelic acid and 0.2 g of caffeine, giving a simple “acid paste” with nearly 10% mandelic acid and 4% caffeine. Elevated temperature (generally above 60° C.) was required to dissolve all of the solids. If used as roughly ⅓ the mass of a deodorant stick with additional lipids, silicones, and other ingredients, the final product, would have over 3% mandelic acid and 1% caffeine and be essentially water free. Thus, the acid paste may comprise a polar organic solvent such as propane diol or propylene carbonate and optional water comprising mandelic acid, optionally additional thickening agents, optionally caffeine, optionally basic salts to adjust pH, and other agents as needed.

“Relatively more viscous than water” as used herein to describe the acid starch indicates that it is at least 5 times, 10 times, 50 times, 100 times, or 500 times more viscous than water. Alternatively, the measured viscosity may be at least 5, 10, 50, 100, or 500 centipoise (cps), such as from 5 to 100,000 cps, from 10 to 50,000 cps, from 100 to 25,000 cps, from 100 to 5,000 cps, from 50 to 5000 cps, etc. The viscosity can be measured at 25° C., or, alternatively, at 60° C. or 70° C., with a Brookfield viscometer operating at 20 RPM. For an acid paste comprising starch or other solids not yet dissolved, viscosity should be measured only after the acid paste has first been heated sufficiently (e.g., to at least 70° C. or 75° C.) to gelatinize the starch or to promote more complete dissolving of solids before bringing the acid paste to the chosen temperature for viscosity measurement. Unless otherwise specified, the temperature for viscosity measurement of the paste can be taken as 70° C., which can in many aspects also be a useful temperature for blending the aqueous phase with the oil phase and/or silicone phase, if present. Without wishing to be bound by theory, it is believed that the elevated viscosity of the aqueous phase relative to water assists in the blending of the aqueous phase with the relatively more viscous oil and/or silicone phases (including an oil phase already comprising silicone compounds), and/or promotes stability of the resulting emulsion or mixture. The viscosity of the acid paste (aqueous phase) may be measured using a Brookfield viscometer, model RVF, at 20 rpm, following directions at https://www.brookfieldengineering.com/-/media/ametekbrookfield/manuals/obsolete %20manuals/dial %20m85-150-p700.pdf?la=en.

As used herein, a “thickener” is an agent that can substantially increase the viscosity of a solution of mandelic acid in a solvent. Thickeners may include starches such as native starches, modified starches, cold-water soluble starches, and the like, including but not limited to corn starch, tapioca starch, potato starch, cassava starch, arrowroot starch, wheat flour, sago, cationic starches such as cationic corn starch, etc. Thickeners may also include gums such as xanthan gum, guar gum, Sclerotium gum, locust bean gum, acacia gum, konjac gels, alginin and its derivatives, namely, alginic acid, sodium alginate, potassium alginate, ammonium alginate, and calcium alginate, and the like. Polysaccharide gums and other polysaccharide thickeners may be used such as pullulan, pectin, agar, gelatin, and carrageenan (both kappa and iota forms) can be considered. Cellulose derivatives may also be used such as cellulose ether derivatives such as carboxymethyl cellulose, methyl cellulose, ethyl cellulose, ethylmethyl cellulose, hydroxyethyl cellulose, hydroxymethyl cellulose, hydroxyethylmethyl cellulose, hydroxypropyl cellulose, hydroxypropyl methyl cellulose, hydroxypropyl ethyl cellulose, hydroxyalkylcellulose polymers, and the like. Mineral agents may be used such as slurries of clay materials such as kaolin, hectorite, thickening waxes sold for cosmetic purposes, betonite, laponite, silica, alumina, attapulgite, montmorillonite, hydroxyapatite, talc, etc., and mixtures or derivatives thereof. Various polymers may also be used such as polyvinyl alcohol, polyacrylic compounds such as carbomer, polylactic acid, carboxomer polymers, various superabsorbent polymers, and the like. Viscous polyols such as those having a viscosity at least 5 times or at least 50 times that of water may be considered. In some aspects, however, the thickener itself may be substantially free of polyols, though polyols may be present as a solvent.

As used herein, “polyols,” also known as polyhydric alcohols, are defined as organic compounds having at least two hydroxyl groups per molecule. The general formula of the suitable polyols are: R(OH)ₙ where n is equal to or greater than 2 and R is generally C2-C10 alkyl or substituted alkyl group. Suitable polyols may include glycerin (also known as glycerol), propylene glycol (also known as 1,2-propanediol), 1,3-propanediol, 1,2-butanediol, 1,3-butanediol, 1,2-pentanediol, 1,5-pentanediol, isopentyldiol, 1,2-hexanediol, 1,6-hexanediol, diethylene glycol, diglycerin, dipropylene glycol, triethylene glycol, 1,2,3-hexanetriol, 1,2,6-hexanetriol, or combinations of the suitable polyols in any given ratio. 1,6-Hexanediol, also known as hexamethylene glycol, is a solid at room temperature (melting point: 42.8° C.) and may also be considered. In general, any alkyl diol having from 3 to 9 carbons and a viscosity at 20° C. of at least 20 mPa-s and more specifically any 1,n-alkanediols for n less than 9 may be considered such as methylpropanediol. Liquid alkyl triols may be considered such as butanetriol. Esters of alkyl glycols (mono- and diglicerides, for example) having up to 7 carbons esterified with carboxylic acids or fatty acids, the acid having up to 8 carbons, may be considered, including, for example, neopentyl glycol diheptanoate, provided the melting point of the ester is no higher than 80° C., 70° C., 60° C., 50° C., 40° C., 30° C., or 20° C. In some aspects, the thickener can act as a solvent. In some such aspects, the thickener, when heated to 70° C. in substantially pure form, can dissolve mandelic acid powder at a level of at least 20 g, 15 g, 10 g or 5 g per 100 g of thickener. Polyols, whether used as a thickener or a solvent or both, may be present at levels such as 0.3% to 25%, 0.3% to 20%, 1% to 25%, 0.5% to 15%, 0.5% to 10%, 0.2% to 6%, 0.5% to 5%, or 0.5% to 3%, or less than 3%.

As used herein, “lipids” include fatty acids or their derivatives such as those commonly used in cosmetics, including fats, oils, fatty alcohols, fatty esters, etc. Lipids may be either polar or non-polar lipids or combinations thereof. Example of polar and non-polar lipids and means for distinguishing the two are described in U.S. Pat. No. 9,084,734, “Peptide personal care compositions and methods for their use,” issued to K. D. Collier et al., Jul. 21, 2015. As used herein, lipids are regarded as “polar,” if their interfacial tension toward water is less than 30 mN/m.

As used herein, “emollients” are compounds that tend to lubricate the skin, increase the smoothness and suppleness of the skin, prevent or relieve dryness of the skin and/or protect the skin. Emollients are typically water-immiscible, oily or waxy materials and emollients can confer aesthetic properties to a topical composition. Emollients may include: 1) Straight and branched chain hydrocarbons having from about 7 to about 40 carbon atoms, such as mineral oils, dodecane, squalane, cholesterol, hydrogenated polyisobutylene, isohexadecane, isoeicosane, isooctahexacontane, isohexapentacontahectane, and the C7-C40 isoparaffins, which are C7-C40 branched hydrocarbons, as well as isopentacontaoctactane, petrolatum and mixtures thereof; etc.; 2) fatty esters (fatty acid esters) as described hereafter under “lipids,” 3) C1-C30 mono- and poly-esters of sugars and related materials. derived from a sugar or polyol moiety and one or more carboxylic acid moieties that may be liquid or solid at room temperature, such as glucose tetraoleate, the galactose tetraesters of oleic acid, the sorbitol tetraoleate, sucrose tetraoleate, sucrose pentaoleate, sucrose hexaoleate, sucrose heptaoleate, sucrose octaoleate, sorbitol hexaester, cottonseed oil or soybean oil fatty acid esters of sucrose, etc., 4) vegetable oils, as described hereafter; 5) soluble or colloidally-soluble moisturizing agents such as hyaluronic acid and chondroitin sulfate.

Lipids of use herein may include (1) fatty alcohols such as stearyl alcohol, oleyl alcohol, behenyl alcohol, isostearyl alcohol, cetyl alcohol, myrsityl alcohol, laurel alcohol, erucyl alcohol, palmitoleyl alcohol, arachidyl alcohol and other C12-C36 alcohols; (2) fatty or oil-like esters such as alcohol esters of C1-C30 carboxylic acids and of C2-C30 dicarboxylic acids including straight and branched chain materials as well as aromatic derivatives such as diisopropyl sebacate, diisopropyl adipate, isopropyl myristate, isopropyl palmitate, methyl palmitate, myristyl propionate, 2-ethylhexyl palmitate, isodecyl neopentanoate, di-2-ethylhexyl maleate, cetyl palmitate, myristyl myristate, stearyl stearate, isopropyl stearate, methyl stearate, cetyl stearate, behenyl behenrate, dioctyl maleate, dioctyl sebacate, diisopropyl adipate, cetyl octanoate, diisopropyl dilinoleate, or mono-. di- and tri-glycerides of C1-C30 carboxylic acids, e.g., caprilic/capric triglyceride, etc., alkylene glycol esters of C1-C30 carboxylic acids, e.g., ethylene glycol mono- and di-esters, and propylene glycol mono- and di-esters of C1-C30 carboxylic acids e.g., ethylene glycol distearate, etc.; (3) fatty acids such as stearic acid, palmitic acid, oleic acid, glycerin monostearic acid, glycerin distearic acid, glycerin monooleic acid, myristic acid, isopropyl myristic acid, isopropyl stearic acid, butyl stearic acid, dicapric acid, neopentyl glycol, other C12-C28 acids, and the like; and (4) fatty acid amides, described hereafter. Lipids may include a variety of oils such as vegetable oils and hydrogenated vegetable oils including avocado oil, safflower oil, grapeseed oil, coconut oil, cottonseed oil, menhaden oil, palm kernel oil, palm oil, rapeseed oil, linseed oil, rice bran oil, pine oil, nut oil, sesame oil, sunflower seed oil, partially and fully hydrogenated oils from the foregoing sources and mixtures thereof;

“Polar oils” include those from the group of lecithins and of fatty acid triglycerides, namely the triglycerol esters of saturated and/or unsaturated, branched and/or unbranched alkane carboxylic acids having a chain length of from 8 to 24, in particular 12 to 18, carbon atoms. In some embodiments, the fatty acid triglycerides are chosen from the group consisting of synthetic, semi-synthetic and natural oils (e.g., olive oil, sunflower oil, soya oil, groundnut oil, rapeseed oil, almond oil, palm oil, coconut oil, castor oil, wheatgerm oil, grapeseed oil, thistle oil, evening primrose oil, macadamia nut oil and the like). However, is it not intended that the present invention be limited to compositions that contain particular polar oils. Additional examples of polar oils that find use in the present invention include the group of esters of saturated and/or unsaturated, branched and/or unbranched alkane carboxylic acids having a chain length of from 3 to 30 carbon atoms and saturated and/or unsaturated, branched and/or unbranched alcohols having a chain length of from 3 to 30 carbon atoms, and from the group of esters of aromatic carboxylic acids and saturated and/or unsaturated, branched and/or unbranched alcohols having a chain length of from 3 to 30 carbon atoms. In some embodiments, such ester oils are chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, semi-synthetic and natural mixtures of such esters (e.g., jojoba oil).

As used herein, “wax-like materials” may include fatty acids, fatty alcohols, fatty acid esters and fatty acid amides. Such molecules may have from 8 to 30 carbon atoms, such as from 12 to 28, 12 to 24, 14 to 28, 16 to 28, or 16 to 24 carbon atoms. Illustrative wax-like materials include cetyl alcohol, palmitic acid, isopropyl palmitate, stearyl alcohol, lauryl alcohol, behenamide, the sucrose esters of tallow fatty acids, the mono- and di-fatty acid esters of polyethylene glycol and the like.

As used herein, “water-swellale polymers” are polymers than can swell substantially in water and therefore can contribute to a thickening effect of an aqueous solution, particularly at a pH below about 5 such as from 2.5 to 5. They may be water soluble but need not be. Hydrogel formers are examples of such polymers. Water swellable polymers may include known superabsorbent materials such as polyacrylates and co-polymers or cross-polymers thereof and various cellulose derivatives such as hydroxymethyl cellulose or other water soluble derivatives. Water-swellable polyurethanes as in WO2004029125A1, etc., may be considered.

As used herein, “silicone compounds” include the numerous silicone derivatives used in cosmetic chemistry and in other personal care applications, often for the smooth textural properties they may impart to an emulsion such as a cream or a solid. Such compounds may include siloxanes such as cyclopentasiloxane, dimethicones, alkyl dimethicones, silesequioxanes including powders such as polymethylsilsequioxane, various liquid silicones or silicone oils such as polydimethylsiloxane (dimethicone), polymethylphenylsiloxane (diphenyl-dimethicone), phenyltrimethicone, diphenylsiloxyphenyltrimethicone, amino-modified silicones, epoxy-modified silicones, polyether-modified silicones, alkyl-modified silicones, phenyldimethicone, cyclomethicone (octamethylcyclotetrasiloxane), hexamethylcyclotrisiloxane, poly(methylphenylsiloxane), cetyldimethicone, behenoxydimethicone and so forth, further including the silicone compounds mentioned in US Patent Application No. 20070196309A1 and U.S. Pat. No. 5,972,359, issued Oct. 26, 1999 to M. Sine et al.

Dimethicone copolyols and related dimethicone derivatives may be considered, particularly polyalkylene oxide modified dimethylpolysiloxanes. Dimethicone copolyols include the polyalkylene oxide modified dimethylpolysiloxanes described in U.S. Pat. Nos. 4,919,934, 4,122,029, 4,265,878, and 4,421,769.

The compounds mentioned above with derivatized organic side chains (e.g., polyethoxylated and/or polypropoxylated) may be considered, such as polysiloxane-polyalkyl-polyether copolymers such as cetyldimethicone copolyol (i.e., cetyldimethicone copolyol (and) polyglyceryl-4 isostearate (and) hexyl laurate).

One challenge in silicone usage, particularly with dimethicones and dimethicone derivatives or other silicone oils, is the challenge of getting a stable emulsion with silicone compounds blended with oil or water or especially oil and water mixtures, particularly for silicone levels about roughly 5% of the composition and perhaps especially at low pH. Some of our initial trials revealed that silicone levels above 5% or so could lead to syneresis (sweating) and other stability issues, but it is believed that the inventive use of a suitable thickener combined with the mandelic acid or other alpha-hydroxy acid (e.g., lactic acid, glycolic acid, etc.) can lead to enhanced stability for the mixture and better integration of elevated silicone levels. In some tests, we have successfully integrated over 30% silicone compounds into a mix also comprising lipids and an aqueous phase, which is believed to be unusual. Thus, in some embodiments, there is provided a stable composition comprising lipids (esters and waxes) at 5% to 70% by weight, significant silicone compounds (5% to 40% by weight), and a thickened aqueous phase carrying enough of one or more acids such as NAC or an alpha-hydroxy acid that is solid in its pure form at room temperature such as mandelic acid to provide at least 1% or at least 2% of the acid in the stick but less than 20% water in the stick or less than 12% or 11% water. Likewise, in some embodiments, a method is provided for making such a stick by combining the lipids and silicones in a molten phase, adding at least one of an emulsifier or gelling agent, blending in a thickened aqueous phase comprising the alpha-hydroxy acid to form a low pH molten mass, and optionally adding a powder prior to pouring the molten mass to form a deodorant stick having an effective pH from 2 to 6, or from 2 to 5.5, or from 2.5 to 5.

As used herein, a “semi-solid” refers to a combination of solid and liquid materials or a composite material with multiple phases or discrete components which does not readily flow under the force of gravity when a unit such as a 5-cm cube of the material rests on a flat surface at 20° C., but which can deform and flow under shear. When measured at a shear rate of 0.5 sec⁻¹, the viscosity may be at least 15,000 centistokes, such as from 15,000 to 10,000,000 centistokes, from 30,000 to 10,000,000 centistokes, from 50,000 to 5,000,000 centistokes, or from 80,000 to 5,000,000 centistokes. Commercial deodorant sticks, whether based on waxy material, aqueous gels, or silicone compounds, are commonly semi-solids.

As used herein, “emulsifying wax” refers to waxy materials that promote emulsification of an aqueous or polar phase with an oily, non-polar phase. Emulsifying waxes generally contain compounds derived from fatty acids such as fatty alcohols, esters, and other materials such a Polysorbate 60 or other emulsifiers. Emulsifying waxes may be plant derived, such as derivatives of palm oil, soy oil or olive oil. Examples include the combination of cetearyl alcohol and glyceryl stearate, such as Ritamulse SCG (sometimes known as Emulsimulse) from Rita Corp. (Crystal Lake, Ill.), which is a combination of glyceryl stearate, cetearyl alcohol, and sodium stearoyl lactylate; Emulsifying Wax NF (cetostearyl alcohol and polysorbate 60), and Polawax (cetearyl alcohol, PEG-150 stearate, polysorbate 60, and steareth-20). A useful plant-derived example is Milliard® All Natural Emulsifying Wax (Milliard Brands, Lakewood, N.J.) said to be derived from palm oil, with the INCI name of cetostearyl alcohol and polysorbate 80. Another natural emulsifying wax derived from olive oil is the combination of cetearyl olivate and sorbitan olivate. Emulsifying waxes are generally taught to be used at a level of 1% to 6% or 2 to 5% of the mass of the emulsion being made (e.g., the combination of the oil and aqueous phases).

Likewise, as used herein, “emulsifiers” for W/O emulsions may include but are not limited to the emulsifying waxes mentioned above as well as one or more of the following compounds: glyceryl stearate, sodium stearoyl lactylate, sorbitan olivate, cetearyl olivate, cetearyl glucoside, sodium cetearyl sulfate, lecithin, lanolin, microcrystalline wax (Cera microcristallina) in a mixture with paraffin oil (Paraffinum liquidum), ozokerite, hydrogenated castor oil, polyglyceryl-3 oleate, wool wax acid mixtures, wool wax alcohol mixtures, pentaerythrithyl isostearate, polyglyceryl-3 diisostearate, beeswax (Cera alba) and stearic acid, sodium dihydroxycetylphosphate in a mixture with isopropyl hydroxycetyl ether, methylglucose dioleate, methylglucose dioleate in a mixture with hydroxystearate and beeswax, mineral oil in a mixture with petrolatum and ozokerite and glyceryl oleate and lanolin alcohol, petrolatum in a mixture with ozokerite and hydrogenated castor oil and glyceryl isostearate and polyglyceyl-3 oleate, PEG-7 hydrogenated castor oil, ozokerite and hydrogenated castor oil, polyglyceryl-4 isostearate, polyglyceryl-4 isostearate in a mixture with cetyldimethicone copolyol and hexyl laurate, laurylmethicone copolyol, cetyldimethicone copolyol, acrylate/C10-C30-alkyl acrylate crosspolymer, Poloxamer 101, polyglyceryl-3 dioleate, glyceryl stearate in a mixture with ceteareth-20, ceteareth-25, ceteareth-6 in a mixture with stearyl alcohol, laureth-4 phosphate, stearic acid, propylene glycol stearate SE, PEG-25 hydrogenated castor oil, PEG-54 hydrogenated castor oil, PEG-6 caprylic/capric glycerides, glyceryl oleate in a mixture with propylene glycol, ceteth-2, ceteth-20, polysorbate 60, glyceryl stearate in a mixture with PEG-100 stearate, laureth-4, ceteareth-3, isostearyl glyceryl ether, cetylstearyl alcohol in a mixture with sodium cetylstearyl sulfate, laureth-23, steareth-2, glyceryl stearate in a mixture with PEG-30 stearate, PEG-40 stearate, glycol distearate, PEG-22 dodecyl glycol copolymer, polyglyceryl-2 PEG-4 stearate, ceteareth-20, steareth-20, isosteareth-20, PEG-45/dodecyl glycol copolymer, methoxy-PEG-22/dodecyl glycol copolymer, PEG-20 glyceryl stearate, PEG-8 beeswax, polyglyceryl-2 laurate, isostearyl diglyceryl succinate, stearamidopropyl PG dimonium chloride phosphate, glyceryl stearate SE, ceteth-20, triethyl citrate, ceteareth-12, glyceryl stearate citrate, cetyl phosphate, potassium cetyl phosphate, isosteareth-10, polyglyceryl-2 sesquiisostearate, ceteth-10, oleth-20, isoceteth-20, glyceryl stearate in a mixture with ceteareth-20, ceteareth-12, PEG-30 stearate, PEG-40 stearate, and PEG-100 stearate, as well as combinations such as cetearyl glucoside and sorbitan olivate, etc.

As used herein, “gelling agents” refers to compositions that are useful in forming gels with liquid silicones and/or lipids, particularly waxes and fatty alcohols. Such gelling agents often comprise hectorite or related minerals. For example, a variety of commercial gelling agents for use with cosmetics comprise hectorite blended with triglycerides or other lipids such as the combination of dicaprylyl carbonate with stearalkonium hectorite and propylene carbonate. A variety of gelling agents marketed as Bentone gels may be considered, such as those listed at https://www.essentialingredients.com/productdetail.aspx?CatID=0&FunID=0&Srch=b entone%20gel, including dimethicone (and) disteardimonium hectorite (and) triethyl citrate, or a blend of octyldodecanol, disteardimonium hectorite, and propylene carbonate, and the like. Other gelling agents used in the cosmetics industry include GelMaker® NAT from MakingCosmetics.com, a blend of sodium acrylate/sodium acryloyldimethyl taurate copolymer, C15-19 alkane, sodium acrylates/acryloyldimethyl taurate copolymer (and) mineral oil (and) Trideceth-6, and the like.

As used herein, the “waxy phase” consists of the non-aqueous or non-polar materials that are combined as part of the process for forming the deodorant stick described herein. It may comprise waxes, oils, fatty acids, and related oleophilic materials. Thus, the waxy phase may comprise:
waxes such as beeswax, white beeswax, candelilla wax, carnauba wax, microcrystalline wax, paraffin wax, hydrogenated castor oil, rice bran wax, berry wax, myrica fruit wax, laurel wax, castor wax (hydrogenated castor oil), sunflower wax, rose wax, orange wax, momosa wax, jasmine wax, polyethylene wax and other synthetic waxes such as Synkos (Kostor Keunen, Waterford, Conn.), etc.;
alcohols such as stearyl alcohol, behenyl alcohol, cetyl alcohol, cetearyl alcohol, isostearyl alcohol, lauryl alcohol, oleyl alcohol, caprylic alcohol, myristyl alcohol, polyols such as glycerin, glycols such as pentylene glycol, and the like;
fatty esters such as isopropyl myristate, isopropyl palmitate: Used in cosmetics both as a thickening agent and emollient, glyceryl stearate, tridecyl trimellitate, jojoba esters, pentaerythrityl tetraisostearate and other pentaerythrityl tetraesters, lauryl laurate, cetyl esters, PEG stearate, etc.;
oils such as Helianthus annus (sunflower) seed oil, soybean oil, almond oil, walnut oil, coconut oil, caprylic/capric triglicerides, MCT oil, jojoba oil, castor oil, avocado oil, etc.;
fatty acids such as stearic acid, palmitic acid, lauric acid, oleic acid, myristic acid, palmitoleic acid, etc.;
butters such as shea butter, Mangifera indica (mango) seed butter, etc.; and
soaps (metal salts of fatty acids) such as zinc stearate, magnesium stearate, magnesium myristate, sodium oleate, etc.

As used herein, “slip modifiers” are texturizing agents that create an increased sense of slip when a solidified product slides against human skin. Such agents may include, without limitation, caprylyl dimethicone, polymethylsilsesquioxane, aluminum silicate, silica, alumina, talc powder, boron nitride, microcrystalline cellulose, kaolin powder, magnesium aluminum silicate, magnesium silicate, magnesium trisilicate, oleamide, polytetrafluoroethylene, veegum, zinc laurate, zinc myristate, zinc palmitate, zinc resinate, and zinc stearate.

As used herein, “sunblock agents” are compounds effective in absorbing UV light to protect human or animal skin, such as oxybenzone, avobenzone, homosalate, octinoxate, octisalate, octocrylene, zinc oxide, and titanium dioxide.

### BRIEF DESCRIPTION OF THE DRAWINGS

FIG. 1 depicts a product comprising a stick of a material described herein.

FIG. 2 depicts a wipe system 26 for applying compositions according to certain aspects of the products and methods herein.

FIG. 3 is a flow chart showing one aspect of a method for making a stick composition for various products described herein.

FIG. 4 depicts a roll-on dispenser for applying materials described herein to various surfaces.

FIG. 5 depicts a container comprising first and second wells holding ingredients useful in cosmetic or cleaning applications, including NAC.

FIG. 6 depicts an assembly with containers holding various components of a cleaning system for pretreating and treating a surface.

FIG. 7 depicts an assembly for a two-part cleaning system comprising at least one biofilm attack agent.

FIG. 8 depicts a spray bottle containing a biofilm attack agent.

### DETAILED DESCRIPTION

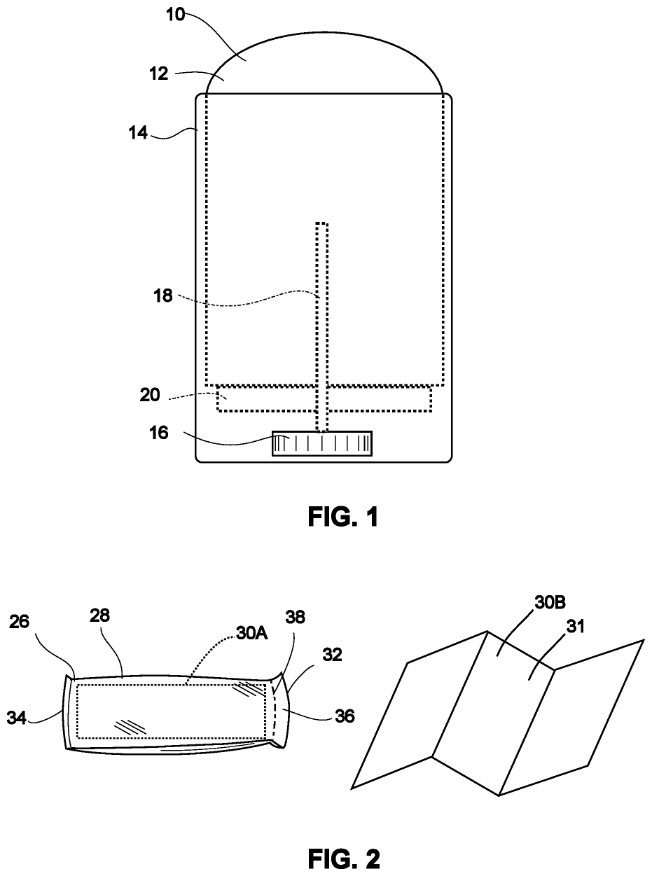

FIG. 1 depicts a product 10 comprising a stick 12 of a material such as a cosmetic material, personal care material, health care material, cleaning material, etc., such a cosmetic material including any one of a deodorant or antiperspirant composition, a lip balm, a sunblock, a wound treatment material, an antifungal or antibacterial composition, a soap or cleaning agent, a material for scrubbing on textiles or various hard or porous surfaces, etc. The stick 12 is contained within the walls of a barrel 44 with an upper opening from which the stick 42 may be dispensed (e.g., pushed out). In the lower portion of the product 10 there is disposed a turnable knob 16 attached to a threaded spindle 18 (threads not shown) that drives the motion of an associated platform 20 on which the stick 12 rests. (Any other position adjustment device may be used, include ratchet mechanisms, manually adjusted sliding tubes, various screw mechanisms, etc.) Thus, by turning the knob 16, the amount of the stick 12 rising above the walls of a container 14 can be adjusted to a selected height above the barrel 14. The stick 12 has an upper surface that is generally dome shaped for comfortable application to the body of the user. (A cap for the product 10 is not shown.)

The barrel 14 may be filled by pouring or injecting a slurry or melt into the barrel 14 and allowing the slurry to harden, or may be formed by packing solid or semi-solid material into the barrel 44 and then compressing it and/or heating it to form a suitable stick 12, which may be uniform or have a gradient in its chemical composition, including discrete regions of differing materials.

FIG. 2 depicts a wipe system 26 for applying compositions according to certain aspects of the products and methods herein. The wipe system 26 comprises an openable pouch 28, which may be a polymeric, foil, paper, or composite pouch, within which is a folded wipe 30A impregnated or coated with a quantity of any one of the NAC-containing compositions described herein, such as a skin care product, a wound treatment, an anti-infection agent, a cleaning product, and/or a biofilm treatment agent. The pouch 28 is shown to have a first end 34 and a second end 36 which may be opened, for example, by tearing the pouch along a tear line 38. An unfolded wipe 30B is also shown, having a surface 31 which may be preferentially treated with the NAC-containing compounds.

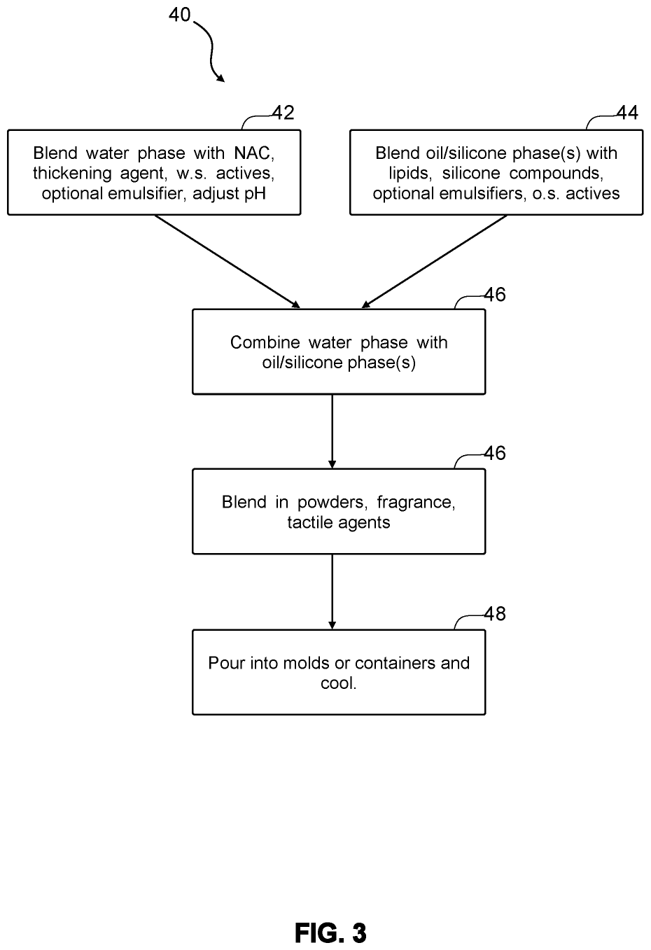

FIG. 3 is a flow chart showing one aspect of a method 40 for making a stick composition for various products described herein. In step 42, a water phase as prepared by blending water with NAC, one or more thickening agents, water soluble actives for the intended function (e.g., Vitamin C, mandelic acid, antimicrobial agents such as cationic steroidal antimicrobials, sunblock agents, anti-wrinkle agents, etc.), optional emulsifiers, pH control agents, thickeners, etc. In step 44, one or more non-aqueous phases such as oil and/or silicone phases are prepared, which may include various lipids, silicone compounds, optional emulsifiers, oil soluble actives, gelling agents, etc. The water phase from step 42 and the non-aqueous phases from step 44 are then combined in step 46, which may include operations such as heating both phases to desired temperatures, blending by mechanical stirring, sonification, use of a homogenizer (rotary, high pressure, ultrasonic), ultrasonification, static mixers, screw extruders, etc. After combination in step 46, step 47 comprising blending in of other ingredients such as powders, fragrance, tactile agents, colorants, heat-sensitive or shear-sensitive ingredients, etc. Following step 47, composition from step 46 is then poured into molds or containers and cooled in step 48.

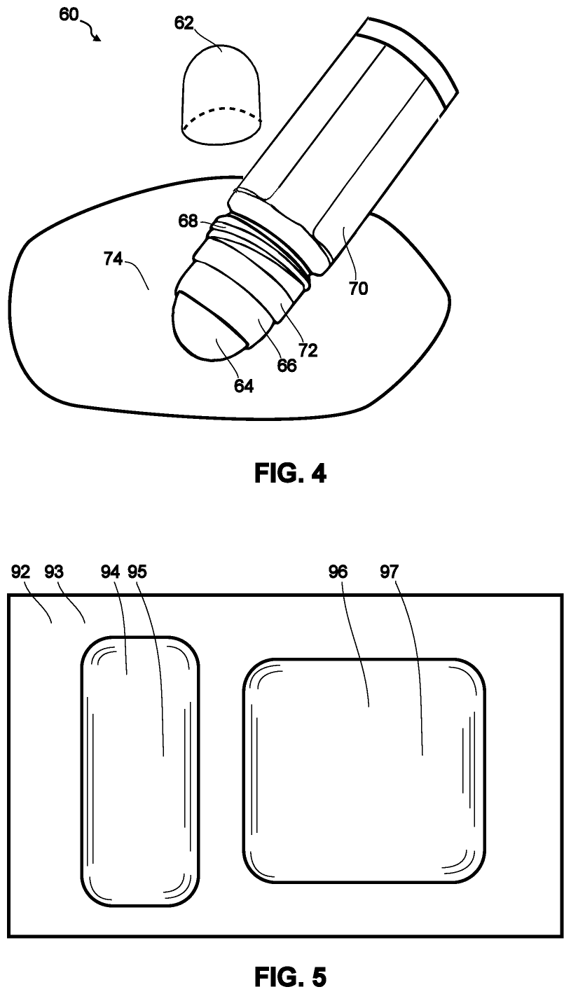

FIG. 4 depicts a roll-on dispenser 60 comprising a cap 62 and a dispenser body 70 containing either (1) an enzymatic mix with water and other agents comprising NAC, (2) N enzymatic mix coupled with a biofilm attack agent such as at least one of NAC, a flavanol solution, and a lysozyme inside (not shown), (3) a biofilm attack agent such as NAC without laundering enzymes in a suitable carrier such as a base of water and optionally a glycol, diol, or suitable solvent and surfactants or other agents, or a combination of any of the above, that can be dispensed using a roll-on ball 64, held in place with a roller body 66 which fits into the upper end 72 of the dispenser body 70. The cap 62 attaches to the dispenser body 70 by engaging threads 68 thereon when it is twisted in the proper direction. The roll-on dispenser 60 can be used to apply a solution to skin or other surfaces 74 such as solid surfaces for household cleaning or to textiles including clothing prior to laundering or directly onto clothing after laundering or between washes. The contents may be refilled by opening a bottom cap (not shown) or unscrewing the roller body 66 or other portion of the dispenser 60.

FIG. 5 depicts a container 92 comprising a first well 94 and a second well 96 which are surrounded by a containment surface 93 which may be a flat or contoured solid surface with a closure or lid (not shown). In one aspect, the container is intended for cosmetic use. In that case, the first well 94 comprises a first composition 95 such as a foundation, cleanser, or moisturizer that can be applied to the skin, while the second well 96 comprises a second composition 97 such as a cream, sunblock, anti-microbial agent, colored cosmetic, etc. The first and second compositions 95, 97 may be intended for different usage such as broad application for one and while the other may be intended for application to a subset of regions for touch-up or other purposes. In one aspect, the first composition is an anti-wrinkle agent or Vitamin C serum that comprises 0.1% or more NAC, while the second composition may be a powdery or mineral-based pigment, or, alternatively, a sunblock agent and/or moisturizer that may also comprise at least 0.1% NAC. The container may be small enough to serve as a single-dose unit, or may be suitable for many uses. Application of the first and second compositions 95, 97 may be done by common or different means such as by use of an applicator comprising foam, cotton, a textile, etc., direct use of fingers, use of a wipe or tissue (not shown), etc.

In an alternative aspect, the container 92 is intended to support cleaning functions, and the first composition 95 may then comprise a paste, solid powder, slurry, or solution comprising cleaning agents such as enzymes and detergents, while the second composition 97 may comprise NAC powder or solution, coupled with other agents such as panthenol. The first and second compositions 95, 97 are thus separated until ready to use, and then can both be combined with a quantity of water to create a cleaning mixture (not shown). In one aspect, the container 92 is water soluble such as a water soluble film such that placing the container in water causes the container and the first and second compositions 95, 97 to dissolve and become available for use in a cleaning solution (not shown). In one aspect, the second composition 97 may be a solid such as a powder or solid capsule comprising NAC and optionally other biofilm attack agents, antimicrobials, buffering agents, cleaning agents such as borax, boric acid, borax, sodium carbonate peroxyhydrate, etc., and first composition 95 may comprise enzymes, surfactants, bacterial spores, with suitable chelants, solvents, rheology modifiers, builders, and the like. In one aspect, the entire container 92 or just the opened and emptied contents of the first and second wells 94, 96 may be placed in a washing machine (for either laundry or dishes, etc.) and run in a wash cycle with or without other items placed therein to reduce biofilm matter in the washing machine (not shown). The mass of each segregated material may range from 0.5 g to 200 g or greater, as needed.

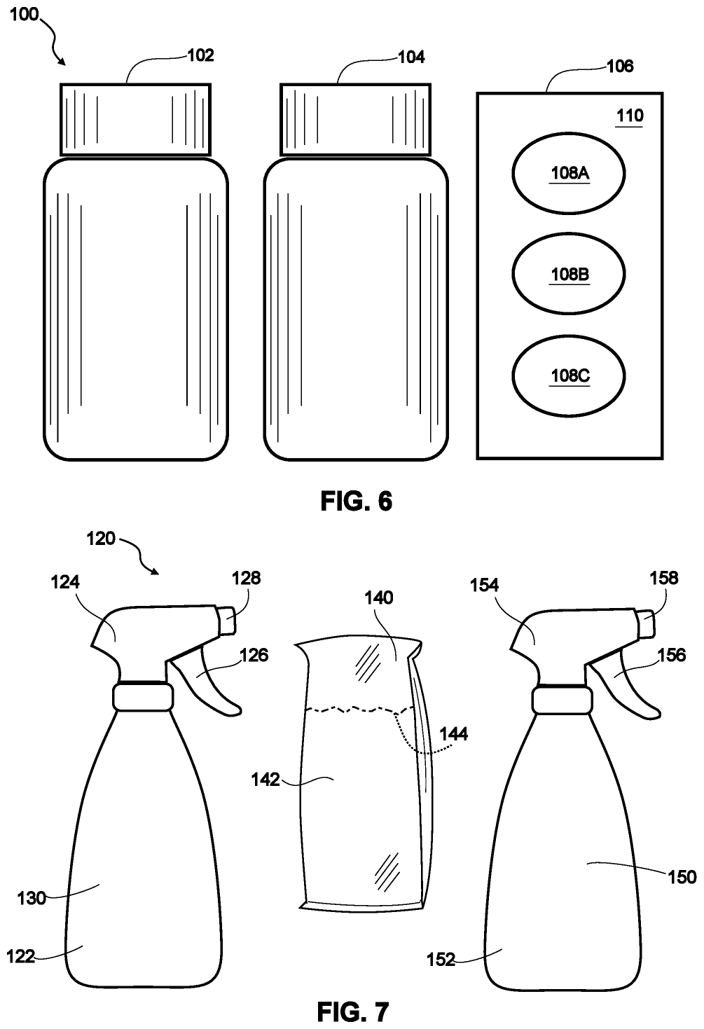

FIG. 6 depicts an assembly 100 comprising a first container 102 with a comprising an enzyme and/or detergent and/or bacterial spores for use as a pretreatment of a surface or item, and a second container 104 comprising a second cleaning agent which may also comprise one or more enzymes and detergents to be applied after the pretreatment. NAC may also be present in either or both of the containers 102, 104. The assembly may further comprise a third container 106 which may comprise a blister pack 110 or other packaging system for providing solids such as three tablets 108A, 108B, and 108C. The tablets 108A, 108B, 108C can be detached from the blister pack 110 and placed in a washing machine to run a sanitizing cycle, in which the antimicrobial ingredients of the tablets 108A, 108B, 108C can be effective in reducing microbes dwelling in the washing machine (not shown). Alternatively, the tablets 108A, 108B, 108C may be placed in a toilet tank or bowl to reduce biofilm growth and may provide controlled release of NAC and other anti-biofilm agents. The contents of the first and second containers 102, 104 may be used in a pretreatment and then cleaning treatment of solid surfaces in a bathroom or kitchen, for example, or of clothing items, etc.

FIG. 7 depicts an assembly 120 for a two-part cleaning system. A first container 122, a first cleaning spray, holds an enzymatic mix comprising one or more surfactants, optional bacteria or bacterial spores such as Bacillus subtilis, and a mixture of three or more classes of enzymes comprising protease and at least two of lipase, amylase, cellulase, mannanase, and pectinase having a total protein content of at least 1% such as from 1% to 15% or from 2% to 10% or from 1% to 8%. The first container 122 is in the form of a trigger spray bottle having a trigger spray mechanism 124 with a trigger 126 operable by one or more fingers and a spray nozzle 128 such as an adjustable nozzle with multiple settings (e.g., jet, spray, on, off) which may have a foaming screen over the nozzle to promote foam formation which in some cases may help reduce the risk of creating minute aerosol droplets of enzymes that might be inhaled. Also shown is a packet 140 containing a powder 144 that may comprise NAC and/or other biofilm attack agents. The packet 140 may be torn open and emptied into a second container 152 or, when made from a water soluble film, may simply be placed in the second container 152 with water, where it will dissolve and can be applied via the trigger spray mechanism 154 with its trigger 156 and nozzle 158 in liquid communication with the contents of the second container 152. The biofilm attack powder may comprise at least 4% or at least 10% NAC such as from 10% to 80% NAC or substantially pure NAC only. It may further comprise from 0.1% to 50% panthenol such as from 1% to 35%, 1% to 25%, 1% to 10% or 1% to 5% panthenol or derivatives thereof. It may further comprise agents to adjust pH, ionic strength, viscosity, wetting angle, rheology, aroma, etc., such as metal salts including magnesium hydroxide, magnesium oxide, magnesium sulfate or citrate, magnesium acetate or chloride, sodium chloride or citrate, sodium bicarbonate or carbonate, potassium chloride or hydroxide, etc., wherein the metal salts may comprise from 0.05% to 20% of the composition. In one aspect, the composition may comprise from 0.05 to 15% each or from 0.1% to 10% each or from 0.3% to 6% each of one or more magnesium salts and one or more sodium salts. The pH of the biofilm attack solution when diluted according to instructions may be from 2 to 10, such as an acidic formulation with a pH range of from 2.5 to 6.5, from 3 to 5.5, or from 3 to 4.8, or less than 4.5. Other pH ranges contemplated include from 3 to 9, 4 to 9, 5 to 9, 6 to 9 and 7 to 9, or from 3.5 to 8.5 or 4 to 9. In another version, the biofilm attack composition 144 comprises caffeine, such as from 0.5% to 12%, or from 1% to 5% caffeine. Caffeine may have an inhibitory effect on biofilm formation.

For aspects in which a solid material such as an NAC-containing mixture is combined with water to make a biofilm attack agent, the packaging may comprise loose powder in a container that is scooped or metered to be combined with water in a container. Alternatively, solid particles may be combined in a tablet that can be dissolved in water, including an effervescent tablet that with NAC and a carbon-dioxide releasing material such as sodium carbonate. In another aspect, the solid particles may be provided in capsules with water-soluble shells that can be dropped into water to form a biofilm attack solution. In a related aspect, capsule shells can be discarded rather than dissolved. “Sprinkle capsules” (not shown) may be used in which large capsules can be gripped and twisted to cause separation and release of contents. Such capsules are described in U.S. patent Ser. No. 10/610,490. They may be made of insoluble material such as polypropylene (PP), PET, high density poly ethylene material, metal, aluminum, and glass.

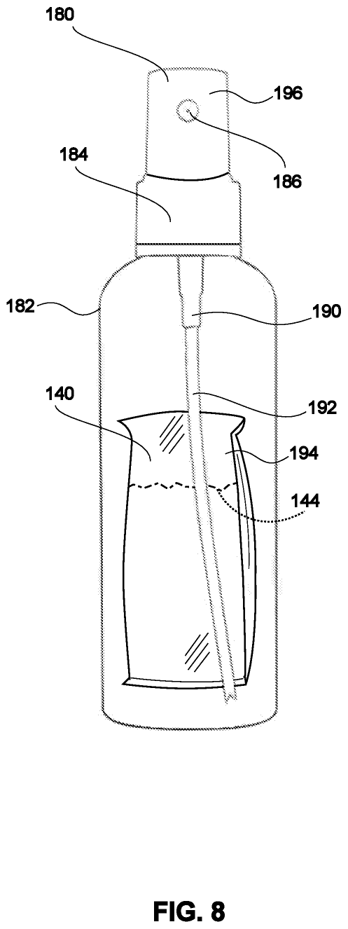

FIG. 8 depicts a spray bottle 180 containing a biofilm attack agent shown as a powder 144 inside a packet 140. The packet 140 is made from a water soluble film 194 such that when water is added to the spray bottle 180, the film 194 dissolves allowing the internal powder 144 to also dissolve and turn the water into a biofilm attack solution (not shown). The bottle 180 comprises a spray bottle body 182, a cap 184 having a nozzle 186 and a spray button 196, from which descends a dip tube connector 190 joined to a dip tube 192 descending into the interior of the spray bottle body 182, providing fluid communication between any fluid (not shown) inside the spray bottle body 182 with the spray nozzle 186, wherein depressing the spray button 196 causes pumping of the liquid through the dip tube 192 to the nozzle 186.

### Surfactants

Surfactants useful in cosmetic and other products include anionic, cationic, amphoteric, and non-ionic surfactants. Anionic surfactants may include, for example, sodium lauryl sulfate (SLS) and ammonium lauryl sulfate (ALS) or ethoxylated versions thereof, as well as sodium laureth sulfate (SLES), etc., and sulfonic acid surfactants such as taurates, isethionates, olefin sulfonates, sulfosuccinates, etc. Cationic surfactants may include quaternized ammonium compounds such as cetrimonium chloride and stearalkonium chloride, amines, alkylimidazolines, alkoxylated amines, etc. Amphoteric surfactants may include sodium lauriminodipropionate, disodium lauroamphodiacetate, etc. Non-ionic surfactants may include alcohols such as cetyl or stearyl alcohol, alkanolam ides, esters, and amine oxides such as cocamidopropylamine oxide and polysorbate esters.

### Enzymes

As used herein, “laundering enzymes” include those commonly used in laundry detergents, both liquid and granulated detergents, such as lipase, cellulase, mannanase, protease, pectinase, and amylase. These are often engineered to be active at an alkaline pH such as from 7 to 9.5 or 7.5 to 8.5 but may individually or collectively be adapted for optimum performance in other pH ranges such as from 3 to 12, 3 to 6, 4 to 7, 5 to 8, 6 to 8, 4.5 to 8.5, 5 to 7.6, 3.5 to 6.5, etc. Enzymes may be incorporated in a product such as a cleaning product at levels from 0.01% to 20% of active enzyme by weight, or from 1% to 15%, 2% to 12%, and the like.

### Skin Care Products and Serums

A variety of skin care serums can be produced using NAC. A serum may comprise 0.1% to 25%, 0.1% to 10%, 0.15% to 5%, or 0.5% to 4% NAC as an antioxidant and microbiome control agent, coupled with one or more of: a) other antioxidants such as natural plant extracts including bakuchiol, Swiss Alpine rose or retinol, coffee see extract, resveratrol or derivatives thereof such as dihydro-resveratrol, plant metabolites such as equol, flavanols, etc., antioxidant or antimicrobial extractives such as multi-fermentation products such as those of Tricholoma matsutake and/or Hericium erinaceum, or those comprising L-ergothioneine; b) peptides such as tripeptides, pentapeptides, etc., c) amino acids and derivatives thereof, such L-cysteine, hydrochlorides of amino acids such a cysteine or cysteine, d) NAC-odor control agents aids for reducing the odor of NAC, typically in water as a solvent, e) solvents such as glycerine, propane diol, ethyoxydiglycol, propylene glycol, etc., f) emulsifiers or stabilizers such as laureth-23, g) skin care agents such as hyaluronic acid, and so forth. Vitamin C serums are of particular interest, but face difficulty due to the tendency for Vitamin C to oxidize if the pH is too high or its difficulty in penetrating the skin. It is believed that the NAC in combination with panthenol and/or skin permeability enhancers such as propane diol may be able to enhance Vitamin C serums through NAC's antioxidant and pH-lowering capacity, and serums based on NAC in combination with Vitamin C appear to have proven effective in initial testing in terms of the effect on wrinkles, though more extensive testing will be helpful. In some aspects, such serums are substantially free of acrylamide compounds such as polyacrylamide and particularly crosslinked polyacrylamide, which can break down into acrylamide, a carcinogen.

Serums may be substantially free of silicone compounds or lipids, or may have relatively small amounts such as less than 10%, 9%, 7%, 5%, 3%, 2% or 1% combined of lipids and silicones, or of lipids alone or silicones alone. Serums may comprise at least 40%, 50%, 60%, 70% or 80% water, and may comprise from 0.5% to 5% of an NAC odor control agent, which alternatively may have a mass ratio relative to NAC of 0.15 to 3, 0.2 to 2, 0.2 to 1, 0.25 to 1.5, 0.2 to 1.3, or from 0.3 to 1.1.

A wide variety of skin care formulations can be considered with NAC to enhance the skin microbiome, to reduce oxidative damage, or to improve skin health. NAC can not only be useful in enhancing the interaction of Vitamin C with the skin, but is believed to have potential synergy with other agents directed to strengthening the skin, reducing inflammation, reducing oxidation or aging, and reducing wrinkles. Skin care ingredients that may be combined with NAC include:
Ginseng extract and extracts of related species.
Cistanche, the Chinese herbal medicine (suosuo dayun in Mandarin), from Cistanche deserticola (the primary source of Cistanche) or Cistanche Herba and related species from the family Orobanchaceae), including Cistanche ambigua, Cistanche phelypaea, Cistanche salsa, Cistanche sinensis, and Cistanche tubulosa. Cistanche contains echinacoside, a useful glycoside, as does the plant Echinacea, extracts of which may also be combined with NAC. Cistanche also contains the glycoside verbascoside, said to be a protein kinase C inhibitor. Either or both of these glycosides from cistanche or other sources may be present in a skin care formulation at a concentration of from 0.01% to 3% total non-celluolosic and non-chitinous glycosides or from 0.01% to 1% for at least one of verbascoside and echinacoside. Skin penetration enhancers such as panthenol may also be present. The pH may be adjusted from 3 to 8 or from 3 to 6 or from 2.5 to 5 when glycosides are used.
Plant extracts such as Cistanche or those from ginseng, Calendula, Siberian carrot, or yeast extracts that increase gene expression of VE-cadherin or integrin α5, as described in European Patent Application EP3711769, “VE-CADHERIN EXPRESSION PROMOTING AGENT AND/OR INTEGRIN 5 EXPRESSION PROMOTING AGENT,” published by K. Kajiya et al., Sep. 23, 2020.
Soy-derivatives such as equol or isoflavones such as daidzein (glycoside: daidzin) and genistein (glycoside: genistin).

Glycosides in general may include partial hydrolysates and semi-synthetic derivatives of starch, cellulose, chitin and alginates such as for instance hydroxypropyl starch, dextrins, hydroxyethyl cellulose, carboxymethyl cellulose, methyl cellulose, chitosan or alginic acid hydrolysates. CM-glucan is a carboxymethylated polysaccharide of yeast with photoprotective features. Emulsion stabilizers form when starch is esterified with phosphoric acid. Chitosan is a partial hydrolysate of chitin and used as a hair conditioner, among others. Saponins, glycosides of steroids and triterpens, may be used, including glycyrrhizin (a glycoside of glycyrrhetic), ruscin found in butcher's broom extract, steroidal saponins found in kigelia extracts (sausage tree). Aescin, a mixture of saponins of chestnut, may be used as well as saponins from horsetail extract. Such saponins may help stabilize the superficial capillary blood vessels and tighten the surrounding connective tissue, and may be useful in cosmetic products for skin care, including products reducing wrinkles including those around the eyes, products for the rosacea, couperosis, edema, etc.

### Oral Care Products

For biofilm control, combinations of NAC with other biofilm attack agents or enhancers can be effective, such as the combination of NAC with EGCG and/or panthenol and/or lysozymes or other suitable enzymes, optionally coupled with odor control agents to mitigate the sulfurous odor that can characterize NAC solutions. In particular, without wishing to be bound by theory, we have found benefits with a variety of compounds that may be able to interact with the sulfur groups of NAC or its oxidation products. Agents that may be useful in reducing the negative perception of odor and/or taste may include compounds with phosphonium ions such as hexametaphosphate salts, sultaine derivatives such as hydroxysultaines; betaine compounds such as cocoa betaine; quaternary ammonium compounds; etc.

Films that can be dissolved in the mouth for oral care can also be formulated with NAC optionally coupled with a NAC-odor control agent such as sodium hexametaphosphate (SHMP), and with flavoring agents that most effectively cover the taste and/or odor of NAC such as citrus extract, including sweet orange or other orange extract, lemon-lime, lime, mint, etc. The same applies to toothpaste formulations, which may use SHMP or other NAC-odor control agents at concentrations similar to those of NAC, such that the NAC-odor control agent/NAC ratio on a weight basis is from 0.3 to 3, 0.5 to 2, or 0.6 to 1.6. For example, a toothpaste, mouthwash, or film may comprise from 0.1% to 2% NAC, and the NAC-odor control agent may be within ±50% of the NAC concentration.

### Cleaning Products

It has been discovered that biofilm on a variety of surfaces, including clothing, textiles, and hard surfaces such as those in bathrooms and kitchens, can be weakened, reduced, or largely with the assisting biofilm attack agents such as N-acetyl cysteine in combination with other agents such as enzymes and/or surfactants. For mitigating “perms-odor” in clothing, especially in synthetic fabrics and sports apparel, biofilm attack agents coupled with enzymes can be applied for an effective period of time, followed laundering with a laundry detergent, treatment with other soaps and detergents, or simply rinsing with water. Biofilm attack agents such as N-acetyl cysteine or N-acetyl cysteine combined with panthenol or derivatives thereof may be provided in a solution, either with enzymes or in a separate container, or provided at least partially in solid form such as a capsule, a powder, a tablet, a stick, etc., to be dissolved in a solution before, after or during application to an item.

In describing various versions and aspects of the methods and products disclosed herein, it should be understood that the elements, steps, features, etc. of any version or aspect are combinable with any other version or aspect or collection of versions and aspects unless stated otherwise or clearly unsuitable.

Thus, in one aspect, a method is provided for treating a solid material with biofilm attack agents in combination with cleaning agents such as enzymes and/or surfactants. The solid material can be a fibrous material including textiles, items of clothing, woven and nonwoven materials or combinations thereof, etc., or hard surfaces such as surfaces of a toilet, sink, floor, shower stall, drains, faucets, refriger-ators, light switches, walls, medical equipment, water fountains, food and drink dispensers, cutting boards, cutlery and utensils in a kitchen, wherein the material is suspected of having microbial biofilm matter in one or more regions that may be associated with persistent odor or other symptoms, or used in an environment or application at risk of developing biofilm and/or persistent odor, the method comprising applying an enzymatic composition to the one or more regions of the solid material, providing suitable time for the enzymatic mixture to attack biofilm, and then washing the textile item, wherein the enzymatic composition comprises: (a) water, (b) from 5% to 60% of a surfactant, (c) from 1% to 20% of an enzyme mixture comprising at least two of lysozyme, proteinase, amylase, mannanase, lipase, pectinase, DNAse and cellulase; and (d) from 0.1% to 10% of N-acetyl cysteine. The enzymatic composition in some aspects is packaged with indicia instructing a user to wait at least 5, 10, 15, or 30 minutes between applying the enzymatic composition and washing the textile, wherein washing generally comprises washing in water with a laundry detergent but may comprise rinsing without use of further detergents.

### Methods of Manufacture

In some embodiments, a deodorant stick is made using a hot pour process in which a hot melt comprising waxes or other meltable compounds is combined and poured into a product container, where the holt met is then allowed to cool and solidify. The hot pour process may involve a bottom fill, in which the hot melt is poured into an open container from the open top and first begins accumulating at the bottom of the container, with the pour finished when the rising level of the hot melt reaches the top of the container. It may also involve a top fill, in which the hot melts is poured through an opening in the bottom of the container, first accumulating at the top of the container in an inverted position, with the pour being completed when the hot melt level rises to what will be the bottom of the container used by a consumer. Any other filling method can also be used, including the injection of highly viscous or even partially solidified material at a temperature controlled to be near or slightly above the solidification temperature as described, for example, by P. S. Allan et al. in U.S. Pat. No. 6,338,840, “Process and apparatus for the production of a deodorant or antiperspirant composition,” issued Oct. 6, 1999, a process that may reduce problems such as shrinking of the solid as it cools from an essentially completely molten hot state and reduce the time required to cool. Allan et al. also describe a method with plurality of inlets into a screw extruder with a flow rate matched with the flow rate of a mobile composition dispensed by a dispenser into containers or moulds (stick barrels). Also of interest is U.S. Pat. No. 4,688,609, “System including nozzle for injecting molten product into deodorant stick containers,” issued Aug. 25, 1987 to M. Diaz., who teaches a system for automatically dispensing metered amounts of viscous product in assembly-line fashion into each of a series of containers, wherein the temperature of the product is maintained within a preselected range along its path of flow.

Manufacture may involve filling deodorant barrels (molds, canisters, tubes, etc.) with mobile or fluid material prepared in batches or prepared continuously, or prepared in a combination of continuous and batch processing such as blending a continuously produced water phase or powder phase with a molten phase produced in batches as the molten phase flows into a mixer prior to be combined with the continuously produced phase prior to pouring.

Mixing of individual or combined phases can be done in any known matter such as using rotating mixer blades in a tank or other kinetic mixers, static mixers, shakers, etc. For example, a mixer blade rotating at about 50 to about 300 RPM may be used while combining an aqueous phase with an oil or oil-silicone phase. The mixer blade speed may be increased or decreased over time as needed, such as increasing to a speed of 400 RPM or greater, such as 600 RPM, as described by B. A. R. Williams et al. in US Patent Application 20120160365, published Jun. 28, 2012. Rotating blades, wires (like a rotating whisk), impellers, etc., may be used. Temperatures also may be varied, such as using distinct temperatures to prepare distinct phases and then possibly adjusting temperature and optionally mixing conditions as phases are combined, including when adding solids such as powders (e.g., starches, silicone materials, antiperspirants, etc.). Thus, in one embodiment, the aqueous phase may be prepared at a temperature of, say, from 40° C. to 80° C., 50° C. to 95° C., or from 55° C. to 75° C., while the oil phase may be prepared at a temperature of, say, from 60° C. to 100° C., 65° C. to 95° C., or 70° C. to 90° C., and then combined with the aqueous phase and brought to an intermediate temperature such as a temperature within 10° C., 5° C., or 3° C. of the mean of the oil phase and water phase temperatures, or a temperature within 5° C. or 10° C. of the oil phase temperature. After or during such mixing, a solid phase such as powder comprising starch, minerals, antiperspirants, silicone powders, pigments, zeolites, or other materials may be blended into the emulsion to give a combination at a temperature that may be from 60° C. to 95° C. and may be lower or greater than the temperature of the combined oil and water phases. The final pour temperature as pouring begins may be greater than the solidification temperature by 3° C. to 40° C., 3° C. to 30° C., 3° C. to 20° C., 3° C. to 15° C., or 3° C. to 10° C., or at least 3° C., 5° C., or 10° C.

### Dispensers, Containers, and Packaging

Any known containers or dispensers may be used for solid stick forms of the preparations described herein, such as popular commercial dispensers with barrels that may be oval or tubular, typically with a plastic body having an internal chamber with a discharge opening, an internal spindle with screw threads that engage a moveable platform for moving a solid body of deodorant or antiperspirant inside the chamber in response to the motion of a rotatable toothed wheel located outside the chamber that is connected to the spindle to permit rotation of the spindle and thereby drive the moveable platform in a piston-like manner to dispense the deodorant or antiperspirant material. The dispenser also may include at least one actuating button located on the dispenser, having a pusher element for rotating the toothed wheel, and at least one pusher deflector for deflecting the pusher element out of engagement with the toothed wheel, as described in U.S. Pat. No. 6,592,278, “Cream and deodorant dispenser,” issued Jul. 15, 2003 to U. Holthaus. As described by Holthaus, the pusher deflector permits the material to be pushed back into the dispenser after use by disengaging the pusher element with the toothed wheel. In some embodiments, the barrel is made from paper fibers such as laminated paper, wax or polymer-coated paper tubing, or molded pulp.

Packaging or labels on individual containers may comprise indicia indicating, for example, that a cosmetic product is to be applied to the skin such as facial skin for cosmetic purposes, or to regions with wrinkles, etc., and/or to skin free of open wounds or lesions or other signs of serious injury. For cleaning products, indicia may direct the application to solid surfaces, textiles, etc., with directions for the length of time the product should be left on the surface prior to further cleaning.

### Further Details

Other ingredients may be present in the acidic sticks described herein, including Vitamin C derivatives such as sodium ascorbyl phosphate, magnesium ascorbyl phosphate, or other ascorbyl phosphate salts, and the Vitamin C compounds and other ingredients listed in U.S. RE38623, “Stabilization of ascorbic acid, ascorbic acid derivatives and/or extracts containing ascorbic acid for topical use,” issued Oct. 12, 2004 to S. Hernandez et al.; US Appl. No. US20070172436A1, published Jan. 23, 2006 by J. Zhang; and US Patent Application No. 20180071205, “Stable Vitamin C System,” published Mar. 15, 2018 by J. Disalvo. (Note that Vitamin C is actually not a carboxylic acid.) U.S. Pat. Nos. 4,647,672 and 5,149,829 describe stable, 2-polyphosphorylated species of L-ascorbic acid and its stereoisomers. The ascorbate 2-polyphosphate esters and other esters of Vitamin C, such as ascorbyl palmitate, ascorbyl dipalmitate, ascorbyl stearate, and other fatty acid or fatty alcohol esters, etc., may be considered for use herein.

In some embodiments, urea may also be present as a solubility enhancer, but in other embodiments, the product is substantially free of urea as well as urea derivatives such as mono-, di-, tri-, and tetra-subsituted urea compounds, and may also be substantially free of other diamides or of other amides in general, particularly those that are not affirmatively described herein as ingredient candidates.

Products described herein may also comprise various derivatives from fungi and lichens including usnic acid (e.g., from 0.01% to 1.5%), but may also be substantially free of usnic acid and/or substantially free of fungal and/or lichen extracts or derivatives.

Preservatives may be added if desired, or the product may be substantially preservative free. Preservatives may include phenoxyethanol, benzoic acid or salts thereof such as sodium benzoate, tris(N-hydroxyethyl) hexahydrotriazine, etc. In some embodiments, however, no other preservatives are needed because of the antimicrobial nature of NAC and other ingredients

In some embodiments, zeolites, particularly antimicrobial zeolites, may be presents at levels such as from 0.1% to 10%, following concepts discussed in U.S. Pat. No. 5,723,110, “Deodorant cosmetic composition superior in resistance to discoloration and dispersion,” issued Mar. 3, 1998 to T. Yamamoto et al.

### Examples

### Odor Testing and Hand Sanitizers

Solution Y1 was made by combining 0.72 g of sodium hexametaphosphate (SHMP) powder with 2.45 g of NAC powder and 15 ml of water. Solution Y2 was made according to the same formula but with 0.73 g of sodium citrate replacing the SHMP. After sitting covered overnight, the odors were compared. Y2 exhibited a strong sulfurous odor, while the odor of Y1 was greatly reduced.

A solution was made with 98 g water, 4.26 g NAC, and 2.955 g NaHCO₃ at a pH of 4.0. This was split equally between two jars labeled O1 and O2. In O2, 1.00 g of SHMP was added, giving a pH of 4.3. Testing from two parties confirmed that the odor in O2 was significantly less than in O1. Further results are discussed below.

Solution Y1 was then combined with two hand sanitizers to evaluate product odor and remaining odor on the hands. In one example, 23.5 g of Everyone™ Hand Sanitizer Spray, lavender and aloe, was combined with 1.00 g of solution Y1. In spite of the significant NAC content, the product had no discernible sulfurous or NAC-like odor, neither in the spray bottle or on the hands after spraying or after allowing the spray to dry. This route has the potential to deliver the benefits of NAC to the skin without noticeable malodor. It is also believed that the modified formulation with NAC and SHMP helps reduce dryness of the skin from the use of alcohol-based sanitizers.

In another example, 1.00 g of solution Y1 was combined with 4.2 g of a common alcohol-gel hand sanitizer. While the viscosity of the gel was significantly reduced, the result was still reasonably viscous and pleasing to use. While the gel lacked any noteworthy fragrance, with the large amount of added NAC there was still no noticeable sulfur smell. SHMP as an NAC-odor control agent may be especially suited for hand sanitizers and related personal care products comprising alcohols.

### Cleaning Examples

Several enzymatic solutions were made, some of which have been described in detail in U.S. Ser. No. 16/926,514, U.S. Ser. No. 17/068,806, and in PCT/US20/55429. First was an enzymatic blend labeled E1, comprised a buffered solution of Novozymes enzymes for laundry detergent in a buffered solution with surfactants and bacterial spores from J-Zyme™ AB-20X NFC distributed by J Tech Sales (Boca Raton, Fla.), said to employ spores from Nozozymes. The solution comprised about 20% J-Zyme which is said to have about 1.1×10⁹ CFU/ml of bacterial spores. This consisted substantially of water, a probiotic bacteria blend believed to comprise Bacillus subtilis spores; enzymes from Novozymes including protease, amylase, pectate lyase, mannanase, 2 types of cellulase, and lipase; alkypolyglucoside from sugar feedstock, sodium citrate, sodium bicarbonate, 1,3 propanediol from natural feedstock, probiotic bacteria blend, and preservative (0.1% of a blend of methylchloroisothiazolinone and methylisothiazolinone). Total enzyme concentration was about 2% by weight. Optimum activity is at a pH of about 7-8.

E2: A blend similar to E1 but without lipase and with the addition of a gentle quat, soyaethyl morpholinium ethosulfate. Ingredients included naturally derived surfactants (from sugar), probiotic bacteria, an enzyme blend containing protease, amylase, pectate lyase, mannanase and cellulases (no lipase); a solvent system made from naturally derived glycerin that also served as an odor control agent, and soyethyl morpholinium ethosulfate. The concentration of the quat was about 0.5% and the enzyme concentration was about 2%.

E3: a blend made from a mix of enzymes, with a total of 5% enzymes comprising pectinase, amylase, mannanase, protease, lipase and cellulase. The solution comprised 20% glucopon-like surfactant from 100% biobased alkyl polyglycosides, sodium citrate and sodium bicarbonate for buffering to a pH in the 7-8 range, propanediol, a mix of bacterial spores approved for bio-enzymatic cleaning from a 10× concentrate comprising Bacillus subtilis spores, a solvent system derived from naturally derived glycerin and as an odor control agent, and a suitable preservative known to be compatible with the bacterial spore mix.

E4C: This blend is a 4:1 concentrate intended upon dilution to give a solution similar to E3, but with slightly reduced surfactant levels. Upon 4:1 dilution, the concentrated E4C solution was diluted to normal strength and dubbed E4D.

E6C is another 4:1 concentrate intended upon dilution to give a solution similar to E3, but with slightly reduced surfactant levels to facilitate the concentrate form and less lipase (0.5% added beyond what was present in the primary blend of Novozymes Leviti Integrate laundering enzymes). This concentrated enzyme blend has about 15% liquid enzyme mixtures comprising pectinase, amylase, mannanase, protease, lipase and cellulase (the liquid enzyme mixtures themselves are estimated to have roughly 40 to 60% protein), about 30% surfactants comprising biobased alkyl polyglycosides, salts such as sodium citrate and sodium bicarbonate, propanediol, a mix of bacterial spores approved for bio-enzymatic cleaning from a 10× concentrate, a solvent system derived from naturally derived glycerin, and a suitable preservative known to be compatible with the bacterial spore mix. Upon dilution (3 parts water to 1 part E6C) the result is E6D (“D” indicates “diluted”).

E6D2: Diluted E6D but only diluted 50% with water. E7C was the same as E6C but with only 0.25% added lipase instead of 0.5%. E8C is the same as E6C but with 0.75% added lipase instead of 0.5%.

PANNAC: 33 ml of 3.6% NAC at pH 4.9 combined with 0.643 g of panthenol powder. PANNAC2: 3.6 g of NAC and 1.65 g of panthenol powder were combined in 108 ml of water, with 2.1 g of NaHCO₃ added to reach a pH of 4.7.

Example C1. A toilet in the house of a large family had recurring biofilm, both with a red color bacteria at the water line in the bowl and apparently a black mold or mildew under the water line. The water level was lowered and the left side of the toilet bowl was wetted with sprays of the PANNAC solution, about 0.6 g applied, allowed to sit for 5 minutes, and then E6D was sprayed across both sides of the toilet bowl and again allowed to sit for about 5 minutes. One fourth of the toilet bowl area (representing roughly the regions associated with a clock at 12, 1, and 9 o'clock) were scrubbed with a melamine foam pad (Magic Eraser®) and the entire toilet bowl was scrubbed with a conventional bristle toilet brush. Ten days later, when the biofilm material would normally have long been entrenched again, the toilet seemed relatively clear, but a few days after that, signs of the biofilm recurred, with extensive biofilm on the right side of the toilet bowl that had been treated with the enzymatic cleaner alone, while the left side was largely free of biofilm except some at the 9 o'clock position (which had been scrubbed with melamine foam). The results suggest that the PANNAC material may have assisted in undermining the biofilm. Several days later, though, the biofilm was back across most of the toilet bowl. In a second treatment, the bowl was drained and NAC powder was sprinkled over the biofilm material. Then dishwashing detergent was applied to the left side and enzymatic toilet cleaner to the right side. After 5 minutes, the bowl was scrubbed with melamine foam, including under the toilet rim where biofilm could be seen, and rinsed.

Example C2. A bathroom sink showed signs of a dark biofilm on the metal ring around the drain. Scrubbing with a coarse pad or melamine pad had little effect. One spray of PANNAC solution was applied to the left side of the ring and 3 sprays of E6D (one spray is about 0.15 g) were applied to the entire ring and allowed to sit for 5 minutes. A melamine pad was then applied to the ring and the biofilm came off readily. It is believed that the PANNAC material had spread around the ring prior to scrubbing. A second sink in the bathroom was resistant to cleaning a similar biofilm ring with a melamine foam pad alone. Some progress occurred after applying E6D for 10 minutes, but more complete cleaning occurred after a second treatment of E4D with a 2 minutes wait before using the melamine pad again.

In further testing with clothing, an artificial sweat mixture, AS-B, described in the related biofilm patent application U.S. Ser. No. 16/926,514, U.S. Ser. No. 17/068,806, and in PCT/US20/55429, was applied to the pits of a black synthetic fiber Walter Hagen shirt that already showed evidence of some biofilm matter under UV light in the pits. The artificial sweat spray was applied to a region near the existing biofilm in hopes of expanding the biofilm zone. The treated shirt was allowed to brew overnight, wrapped in plastic and kept at about 90° F. After washing and viewing in UV light, the biofilm region in a pit had expanded. The process of washing, drying in a tumble dryer, then applying artificial sweat, allowing it to brew overnight, and washing again with a detergent comprising optical brighteners was twice more with slight expansion of the biofilm region.

### Stability and Efficacy of NAC+Enzymes

Testing with combined enzymes and NAC began with a combination of 0.725 g panthenol, 2175 g NAC, 3.4 g water, and taking 1.32 g of the resulting solution and blending it into 22 g of E7C and further adding 0.5 g NaHCO₃ and 2.03 g EBD. This mix is LPN1. Then 2.00 g panthenol, 6.02 g NAC, 6.5 g water and 3.0 g NaHCO₃ were combined and 6.5 g water. After heating and loss of carbon dioxide formed, the mass was 17.5 with 34.4% NAC and the solution was labeled as PNC3. 3.0 g of PNC3 were then combined with 22 g of E7C giving about 1% NAC in the composition, labeled as LPN2. Then 21.6 g of E6C was combined with 2.93 g PNC3 and labeled as LPN3. These mixes would be compared to the original E6C and E7C solutions during extended thermal stability testing at 40° C. for 1, 2, and 3 months. Testing of enzyme activity was done for lipase, protease, and amylase and revealed that LNP and LNP2 had excellent stability, while LPN3 eventually became highly viscous, possibly due to NAC-enzyme interactions at too high a concentration

Measurement of enzyme efficacy was done using pre-stained fabrics purchased from Testfabrics, Inc. (West Pittston, Del.), TestFabrics.com. Protease efficacy was tested using the fabric CFT C-S-101, a cotton fabric stained with dried bovine blood. Amylase was tested using fabric CFT C-S-29, a cotton fabric stained with an orange-colored tapioca starch. Lipase efficacy was tested using fabric CFT P-02, a polyester fabric stained with olive oil and carbon black. In general, testing involved preparing regular sections of the stained cloth, 1 inches by 2 inches, and applying an amount of the enzyme to moisten the cloth and allow it to sit for a fixed period of time at room temperature, followed by immersion of the cloth sections into water for another fixed period of time, and then blotting and allowing the samples to dry, after which the color intensity was measured with a colorimeter. The more effective the enzyme, the more of the stain was removed and the brighter/whiter the fabric sample. The colorimeter was an AMTAST Model AMT599 AMTAST Colorimeter (8MM) sold by Amtast (Lakeland, Fla.). In making measurements, the meter was moved in a regular pattern over cloth samples to make a series of at least 3 measurements that were averaged. Measurements were reported in the L-a-b system, with the lightness value, L, being of most use in the measurements.

For the blood stained cloth, it was necessary to first “set” the blood stain by immersion in hot water to reduce the ability of the blood to diffuse away in water without enzymatic attack. A constant temperature hot water bath was used, set to 65.4° C., and each test sample was first immersed for 5 seconds and then withdrawn and immediately dipped into room temperature water for 5 minutes, then blotted.

About 0.7 g (0.6-0.8 g) of enzyme solution was applied to each sample, typically enough to wick into the sample and wet all or nearly all of the cloth. For the first series of tests after 30 days of aging at 40° C., each sample after application of the enzyme solution was allowed to sit for 15 minutes at room temperature (about 22° C.), and was then immersed into a clean tub of water at 21° C. to soak for 10 minutes. The sample was then withdrawn after a gentle shake under the water and allowed to dry and then L-a-b measurements were recorded on multiple spots to obtain a representative average.

Then lipase testing was done in the same manner with the olive oil stained cloth and the orange stained cloth, following the same protocol except with no need for a heat treatment.

Results from initial protease testing of the blood stained cloth follow in Table 1, where solution 4 is the control, water; 5 is fresh E6D that was not thermally aged and has been fully diluted (3 parts water to 1 part E6C); 6 is aged LPN3, the solution of NAC and E6C; 7 is aged LPN2, the mixture of NAC and E7C; 8 is aged E6C; 9 is aged E7C, and 10 is aged LPN1, a mix of NAC and E7C and EBD. Note that the thermal aging was done to concentrates, but for the testing, a portion of the concentrate was diluted to normal strength (1 part concentrate to 3 parts water) prior to application to the stained cloth. For these early results, the thermal treatment was at a higher temperature than the standard protocol used hereafter and with somewhat less uniformity, resulting in stain that was somewhat harder to remove. A surprising outcome in Table 1 is that LPN2 resulted in a higher brightness gain and thus better removal of blood due to protease activity than the fresh or aged enzymatic mixes themselves.

TABLE 1
Protease testing for aged (30 days, 40° C.) and fresh enzymatic mixes.

| Solution: | 4 | 5 | 6 | 7 | 8 | 9 | 10 |
|---|---|---|---|---|---|---|---|
| Mean L* | 48.66 | 64.41 | 64.14 | 66.18 | 61.55 | 60.78 | 59.62 |
| Mean a* | 15.28 | 8.99 | 8.54 | 8.31 | 9.76 | 9.90 | 9.33 |
| Mean a* | 12.55 | 11.37 | 10.50 | 11.09 | 10.67 | 10.94 | 10.67 |
| Sample st. dev. | 0.42 | 0.55 | 0.58 | 0.50 | 1.10 | 0.22 | 0.78 |
| Conf. int., L* | 0.42 | 0.44 | 0.47 | 0.40 | 0.81 | 0.18 | 0.58 |
| N (# of msrmts) | 4 | 6 | 6 | 6 | 7 | 6 | 7 |

Further protease test results with the proper heat set procedure are shown in Table 2, where Solution 13 is fresh E6D, 14 is aged E6C, 15 is aged E7C, 16 is aged LPN3, 17 is aged LPN1, and 18 is aged LPN2. Again LPN2 outperforms the equally aged enzymatic mixes, and here the other NAC-containing mixtures also perform well. The ability of NAC combined with an enzyme to improve its performance rather than degrade it is truly surprising, and suggests that NAC-protein mixtures, when properly formulated, may have the potential to have improved thermal stability relative to similar compositions without NAC, a highly unexpected finding. Thus, there may be potential for improving the shelf stability or overall performance of a variety of enzyme products, including stain removers, prespotters, laundry detergents, enzymatic cleaners, and other products through the use of a suitable amount of NAC.

TABLE 2
Protease testing for aged (30 days, 40° C.) and fresh enzymatic mixes.

| Solution | 13 | 14 | 15 | 16 | 17 | 18 |
|---|---|---|---|---|---|---|
| Mean L* | 73.59 | 68.49 | 67.33 | 68.54 | 67.62 | 69.10 |
| Sample st. dev. | 0.23 | 0.98 | 0.40 | 0.82 | 1.50 | 0.65 |
| Conf. int., L | 0.23 | 0.96 | 0.39 | 0.80 | 1.47 | 0.64 |
| N | 4 | 4 | 4 | 4 | 4 | 4 |

Similar results were obtained with testing for lipase and amylase efficacy after 30 days, and similar results were also found after 60 days of testing for all 3 enzymes tests based on the removal of stains from prestained fabrics. For testing of lipase based on the olive oil+carbon black stained cloth, results after 30 days of thermal aging were:

TABLE 3
Lipase testing for aged (30 days, 40° C.) and fresh enzymatic mixes.

| Solution | Mean L* | Conf. int. |
|---|---|---|
| Control, water | 56.83 | 0.4 |
| Fresh E6D | 60.67 | 0.4 |
| Aged E7D | 59.8 | 0.36 |
| LPN1, aged | 59.74 | 0.25 |
| Fresh E6D | 60.63 | 0.27 |
| Aged E7D | 60.45 | 0.63 |
| LPN1, aged | 59.55 | 0.21 |
| Aged E6D | 59.82 | 0.55 |
| LPN3, aged | 60.02 | 0.23 |
| LPN2, aged | 60.61 | 1.18 |
| Untreated | 56.59 | 0.28 |

Amylase testing was done on 1.5 in by 2 in sections of cloth. Comparing the efficacy of the aged enzymes to that of the E6D enzyme that had not been thermally, all aged enzymatic mixes experienced a drop in performance as shown in Table 3:

TABLE 3
Loss in amylase efficacy for aged enzyme mixes relative to
unaged E6D.

| LPN1 | LPN2 | LPN3 | E6C | E7C |
|---|---|---|---|---|
| 7.59% | 4.62% | 5.45% | 16.52% | 14.24% |

Thus while the thermal aging decreases amylase efficacy in all samples, those with NAC fared better than those without, again a surprising finding pointing to the potential of NAC-protein combinations for enhanced thermal stability or increased shelf life for low concentrations of NAC (e.g., about 6% or less, 5% or less, 4% or less, 3% or less, or 2% or less, such as from 0.03% to 3% or 0.05% to 1.5%) in a variety of enzymes and perhaps other protein systems in industry and life sciences.

In any case, the possibility clearly exists that NAC can be prepared in solution with laundry enzymes without decreasing their efficacy, and in some cases, may even improve enzyme performance. Thus, it is believed that a variety of the solutions proposed herein, including one- and two-step laundry treatment systems, enhanced laundry detergents, enzymatic cleaners and biofilm removal aids for solid surfaces such as walls, bathroom fixtures, kitchens, etc., may have excellent shelf stability for reasons that go beyond NAC's antimicrobial capabilities.

### Cosmetic Examples

The ingredients mentioned below were generally drawn from the following:
Acrylate copolymer, CAS #129702-02-09, acrylate octylacrylamide copolymer: Making Cosmetics, Inc., Redmond, Wash.
almond oil: Formulator Sample Shop, Iron Station, N.C.
aluminum chlorohydrate powder: Formulator Sample Shop, Iron Station, N.C.
Aminosyl™ L30: sodium lauroyl sarcosinate, Jarchem Industries, Newark, N.J.
Aminosyl™ SCG: sodium cocoyl glutamate, Jarchem Industries, Newark, N.J.
arrowroot starch: Bob's Red Mill, Milwaukie, Oreg.
beeswax: Sky Organics, Delray Beach, FLUORESCENT
behenyl alcohol: Formulator Sample Shop, Iron Station, N.C.
Bentone Gel GTCC V (a gel-making compound comprising pre-dispersed modified hectorite clay in an oleophilic base; INCI name is caprylicc triglyceride and stearalkonium hectorite and propylene carbonate): Elementis, East Windsor, N.J.
C26-28 alkyl dimethicone (Botanisil AD-1): DDChemco, Chatsworth, Calif.
cacao butter: Cacao Butter Wafers, Terrasoul Foods, Fort Worth, Tex.
caffeine: 200 mg capsules, Bulk Supplements, Henderson, Nev.
candelilla wax: TKB Trading, Oakland, Calif.
caprylyl dimethicone (Botanisil CPM-10): DDChemco, Chatsworth, Calif.
caprylic capric triglycerides MCT: Formulator Sample Shop, Iron Station, N.C.
castor wax (hydrogenated castor oil): Health and Beauty Oils Center, Ebay.com
cetyl alcohol: Formulator Sample Shop, Iron Station, N.C.
cocoamidopropyl hydroxysultaine: Making Cosmetics, Inc., Redmond, Wash. (a liquid surfactant used in some baby shampoos due to its gentleness)
coconut oil: LouAna Coconut Oil, Ventura Foods, Brea, Calif.
corn starch: Hodgson Mill, Effingham, Ill.
CreamMaker® Behenyl: Making Cosmetics, Snoqualmie, Wash.
cyclopentasiloxane: TKB Trading, Oakland, Calif.
dimethicone and dimethicone/vinyl dimethicone copolymer (Lotioncrafter EL61): Lotioncrafter, Eastsound, Wash.
DM6 dimethicone (dimethicone with a viscosity of 6 cst): Lotioncrafter, Eastsound, Wash.
DM350 dimethicone (dimethicone with a viscosity of 350 cst): Lotioncrafter, Eastsound, Wash.
diphenyl siloxy phenyltrimethicone (Lotioncrafter LC1550): Lotioncrafter, Eastsound, Wash.
ECOMulse™ (an emulsifier made from glyceryl stearate, cetostearyl alcohol and sodium stearoyl lactylate): Lotioncrafter, Eastsound, Wash.
Ecosil (Fision® EcoSil from Tri-K, a naturally derived blend of hydrogenated ethylhexyl olivate and hydrogenated olive oil unsaponifiables): Formulator Sample Shop, Iron Station, N.C.
Ectoin: IDA Skincare Products Lab, China
emulsifying wax: Milliard® Emulsifying Wax (cetostearyl alcohol and polysorbate 60), Milliard Brands, Lakewood, N.J. (unless otherwise specified, “emulsifying wax” refers to this product)
ethoxydiglycol: Making Cosmetics, Snoqualmie, Wash.
ethylhexyl palmiate: Making Cosmetics, Snoqualmie, Wash.
FSS Sensolv (isoamyl laurate): Formulator Sample Shop, Iron Station, N.C.
GLDA: Tetrasodium Glutamate Diacetate, Jarchem Industries, Newark, N.J.
Hyaluronic acid: SMLW (super-low mol. wt., 8,000-15,000 Daltons) hyaluronic acid, Making Cosmetics, Snoqualmie, Wash.
hydroxpropylcocoate PEG-8 dimethicone (Botanisil TE-3): DDChemco, Chatsworth, Calif.
jojoba oil: Shaoxi Fanglio Studio (Plant Lab), China
Kostol PGP (an emulsifying wax comprising polyglyceryl-3 stearate, beheneth-5): Koster Keunen, Watertown, Conn.
laponite powder: Laponite XL21, BYK USA, Inc., Gonzales, Tex.
lauryl laurate: TKB Trading, Oakland, Calif.
Leucidal SF Max preservative, Formulator Sample Shop, Iron Station, N.C.
lip stick base: TKB Trading, Oakland, Calif. (a mixture of castor seed oil, cetyl stearyl alcohol, olive fruit oil, beeswax, hydrogenated castor oil, glycine soybean lipids, lauryl laurate, carnauba wax, candelilla wax)
magnesium myristate: TKB Trading, Oakland, Calif.
magnesium stearate: MarkNature, location unknown
Olivem 1000 (cetearyl olivate+sorbitan olivate): Hallstar Beauty (Chicago, Ill.).
mandelic acid powder: Pure Health Botanicals, St. Charles, Ill.
panthenol (vitamin B5): L′eternal World LLC, Aurora, Ohio
palmitic acid 98%, Acme-Hardesty, Blue Bell, Pa.
PEG-8 beeswax (esterification of the free fatty acids of beeswax with polyethylene glycol): Koster Keunen, Watertown, Conn.
polyethylene wax: Making Cosmetics, Snoqualmie, Wash.
polymethylsilsesquioxane (Botanisl SP-360): DDChemco, Chatsworth, Calif.
1-3 propanediol (hereafter simply pronanediol): Formulator Sample Shop, Iron Station, N.C.
propylene glycol: Earthborn Elements, American Fork, Utah
shea butter (unrefined): TKB Trading, Oakland, Calif.
Softisan: FSS Softisan 378, Formulator Sample Shop, Iron Station, N.C. (this material is a lanolin-like material made from a blend of triglycerides based on saturated even-numbered, unbranched natural fatty acids of vegetable origin)
soluble starch, product IS28205, CAS #9005-84-9, Aldon Corp., Avon, N.Y.
stearyl alcohol: Alcohol 1989 N F Pastilles, Acme-Hardesty, Blue Bell, Pa.
sunflower wax: Making Cosmetics, Snoqualmie, Wash.
Synkos 2050 wax: Koster Keunen, Waterford, Conn.
tapioca starch: Erawan Marketing Co., Bangkok, Thailand
TKB gelmaker CC: TKB Trading, Oakland, Calif. (this material is a blend of dicaprylyl carbonate, stearalkonium hectorite and propylene carbonate, and is used to create gels)
Vitamin C, buffered powder: Pure Encapsulations (Sudbury, Mass.)
Vitamin C, pure powder: Resurrection Beauty, Holmen, Wis.
water is distilled water unless otherwise specified
xanthan gum: Carrington Farms, Closter, N.J.
zinc stearate: TKB Trading, Oakland, Calif.

Any of these ingredients or those described elsewhere herein can be considered for addition in suitable quantities to anything considered herein.

Unless otherwise stated, the cosmetic stick compositions described below were made in a double boiler constructed by using a muffin baking pan with 4×3 muffin wells and external dimensions at the rim of about 13.9 inches×10.6 inches and a well depth of about 1.2 inches and a well diameter of about 2.75 inches. The muffin pan could fit snugly within a large Wilton® baking pan having internal dimensions near the top of the slightly tapered pan of about 14.4 inches×10.8 inches×2 inches. During formulation work, the baking pan was placed on a gas stove covering two burners, then filled with water to a depth that allowed the muffin pan to float. The burners could then be turned on to bring the water to a suitable temperature for melting wax and other components in one or more of the wells. The muffin pan came with a detached well that had not been welded to the main pan, a manufacturing defect that provided additional convenience since the loose well could serve as a convenient weighing pan and could, when needed, be placed directly inside one of the other wells to melt and mix ingredients, after which the contents could be weighed if desired to see, for example, how much moisture may have evaporated. The open well port allowed an easy way to add water conveniently or to preheat utensils such as whisks or spoons.

Illustrative runs made with significant antiperspirant content are shown in runs P1-P4 below and later in runs P5-P9. For runs P1-P4, ingredients added to the oil-silicone phase are shown in Table 1A, including the “water phase add (addition),” which states how much of the acid paste for each run was combined with the oil-silicone phase. The respective acid paste composition is shown in Table 1B. The overall composition of the final stick is shown in Table 1C.

TABLE 1A
Several antiperspirant runs with oil-silicone phases.

| | Run: | | | |
|---|---|---|---|---|
| Oil phase + silicone phase | P1 | P2 | P3 | P4 |
| aluminum chlorohydrate | 5.5 | 4.2 | 5.03 | 3.994 |
| arrowroot starch | 2 | 3.56 | 3.56 | 4.15 |
| behenyl alcohol | | | 1.03 | |
| Bentone Gel GTCC V | 1.608 | 1.77 | | |
| C26-28 alkyl dimethicone | 1.302 | 1.56 | 1.06 | 1.13 |
| cacao butter | | | | 0.45 |
| caprylyl dimethicone | 1.949 | 0.12 | 1.55 | 0.90 |
| CreamMaker ® Behenyl | | | | 0.69 |
| DM6 dimethicone | 6.03 | 6.63 | 3.93 | 2.28 |
| DM350 dimethicone | 0.85 | 0.94 | 1.72 | 1.00 |
| Ecosil | | | 1.446 | |
| Gelmaker CC | | | 1.15 | 0.67 |
| isoamyl laurate (Sensolv) | 4.083 | 4.326 | 1.737 | 2.489 |
| Jojoba oil | | | 1.526 | 0.455 |
| Kostol PGP | 0.216 | 0.409 | 0.518 | |
| panthenol | 0.251 | | 0.15 | 0.217 |
| polyethylene wax | | | | 2.143 |
| polymethylsilsesquioxane | 2.896 | 2.547 | 2.12 | 2.54 |
| (SP-360) | | | | |
| squalane | | | 0.26 | 0.15 |
| stearyl alcohol | 2.5 | 1.3 | 3.23 | 2.076 |
| Synkos 2050 wax | 8 | 7.59 | 5.261 | 3.686 |
| Acid paste addition: | 7 | 3.95 | 7.17 | 6.05 |
| Water correction (est. evap. loss) | 0 | | 0.1 | 0.05 |
| Total mass (g): | 44.19 | 38.91 | 42.34 | 35.02 |

TABLE 1B
Acid paste compositions corresponding to the “acid paste
addition” listed in Table 1A.

| Water Phase | | | | |
|---|---|---|---|---|
| Composition | AP21 | AP29 | AP30 | AP31 |
| Water | | 10.25 | 2.46 | 0.30 |
| Tapioca Starch | | | 0.00 | |
| Mandelic Acid | 8.89 | 6.90 | 1.65 | 2.80 |
| NAC | | 3.34 | 0.80 | 2.00 |
| Glycerin | | | 0.00 | |
| Propane(1,3)diol | 20.28 | 4.14 | 0.99 | 5.30 |
| Propylene glycol | | | 0.00 | |
| Caffeine | 0.00 | | 0.00 | |
| NaOH | 0.00 | 0.26 | 0.06 | |
| Alum. Chlorohydrate | | 3.90 | 0.93 | |
| Allantoin | | | 0.37 | |
| GLDA | | | 0.13 | |
| Cocoamido Hyd. | | | 1.59 | 0.47 |
| Sultaine | | | | |
| Initial mass: | 29.18 | 28.79 | 8.99 | 10.87 |

TABLE 1C
Overall percentage of ingredients in final sticks.

| Summary of Key | | | | |
|---|---|---|---|---|
| Components: | P1 | P2 | P3 | P4 |
| Aluminum chlorohydrate %: | 12.4% | 12.2% | 13.6% | 11.4% |
| Caffeine %: | 0.00% | 0.00% | 0.00% | 0.00% |
| Mandelic acid %: | 4.83% | 2.43% | 3.12% | 4.45% |
| Silicone compounds %: | 29.5% | 30.3% | 24.5% | 22.4% |
| Synthetitc wax %: | 18.1% | 19.5% | 12.4% | 16.6% |
| Starch %: | 4.53% | 9.15% | 8.41% | 11.8% |
| Water %: | 0.00% | 3.61% | 4.39% | 0.33% |
| Glycerin %: | 0.00% | 0.00% | 0.00% | 0.00% |
| Esters (with triglycerides) %: | 9.2% | 11.1% | 11.7% | 10.1% |
| Fatty alcohols & acids %: | 5.66% | 3.34% | 10.1% | 5.9% |
| NAC: | 0.00% | 1.18% | 1.51% | 3.18% |
| Diol/glycol %: | 11.0% | 1.46% | 1.87% | 8.42% |
| NaOH: | 0.00% | 0.09% | 0.12% | 0.00% |
| Gelling agents, emulsifiers: | 4.13% | 5.60% | 3.94% | 3.88% |
| Panthenol: | 0.57% | 0.00% | 0.35% | 0.62% |
| Allantoin | | 0.00% | 0.70% | 0.00% |
| GLDA | | 0.00% | 0.24% | 0.00% |
| Hydroxysultaine | | | 3.00% | 0.75% |
| Total: | 100% | 100% | 100% | 100% |
| pH: | 3.9 | 3.8 | 3.5 | 3.7 |

After pouring into round deodorant molds, it was observed that the solid sticks had a good feel and a uniform texture. Sticks P1 and P2 were tested on human underarm skin without irritation and no sense of grittiness. The hardness of stick AP2 was measured using an AMS 59032 E-280 Pocket Penetrometer, measured by increasing the applied pressure slowly as the tip engaged the wax until there was a sudden breakthrough and then reading the peak pressure indicated by a movable rubber ring. The units are in kg/sq. cm or tons/sq foot (1 kg/sq cm=1.02 tons/sq foot). A hardness of 1.25 was recorded.

Observations on Reducing the Odor of NAC. These sticks were tested in human use to various degrees. Stick P4 had a noticeable unpleasant smell, apparently due to NAC perhaps particularly in combination with lavender. Stick P3 had no readily detected odor from the NAC, even though one of the more malodorous versions of NAC found on the market was used. Without wishing to be bound by theory, it is proposed that the cocoimidopropyl hydroxysultaine interacted with the NAC, possibly via its amido group, to capture sulfurous impurities or reduce oxidation of NAC to reduce the production of sulfurous odorants over time. After storage for several weeks there was no apparent sulfur smell in the product.

The reason cocoimidopropyl hydroxysultaine was employed in this NAC-containing deodorant was that in trials of odor suppression conducted in Appleton, Wisconsin in support of this investigation, a variety of household products were combined with a relatively strong-smelling NAC solution at 5% concentration, and the mixtures were then examined for odor response. A mixture with an HONEST™ brand baby shampoo containing a large percentage of cocoimidopropyl hydroxysultaine was observed to have an unusually low odor of NAC odor, and thus it was hypothesized that cocoimidopropyl hydroxysultaine and other sultaine compounds may be effective in reducing NAC odor. The particular mixture had 21 ml of water, 1.08 g of NAC, and 2.3 g of Honest Shampoo and Body Wash. Cocamidopropyl hydroxsultaine was the first ingredient after water. It also contained a significant amount of sodium methyl cocoyl taurate. Pure cocoimidopropyl hydroxysultaine was then obtained for runs using it as an ingredient in a deodorant stick. It was also tested at 2% concentration in a 6% NAC solution and appeared to have low NAC odor immediately and still after several days.

Other compounds with amidoalkyl groups of quaternary ammonia groups or both may also be considered for such functions, including lauramidopropyl betaine, cocamidopropyl betaine, other betaine compounds and other sultaine compounds such as lauramidopropyl hydroxysultaine, oleamidopropyl hydroxysultaine, tallowamidopropyl hydroxysultaine, erucamidopropyl hydroxysultaine, lauryl hydroxysultaine, etc. Sodium methyl cocoyl taurate, especially when dispersed in a water phase or in an oil/water emulsion, also may have a helpful role in suppressing NAC odor, and was also tested in some runs described below. In one test, 0.5 g of sodium methyl cocoyl taurate was dispersed in about 20 ml of water comprising 3 g of dissolved NAC and 1 g of cocoamidopropyl hydroxsultaine. After dispersion, the mixture had some bubbles over the solution surface, but these gradually dissipated. Odor reduction of the mix was not as effective as with EDTA, however, and, without wishing to be bound by theory, it may be that the taurate interfered with the cocoamidopropyl hydroxsultaine in suppressing the odor.

Similar tests of various agents blended into NAC solutions at concentrations of 3 to 10% suggest that the following may also be useful in reducing odor, again possibly because of interaction with nitrogen groups in the compounds: GLDA, panthenol, ectoin, sarcosinates such as sodium lauroyl sarcosinate, EDTA, and urea. In some tests, it appeared that calcium ions or compounds such as calcium phosphate could be helpful. For example, 1.0 g of EDTA was combined with 1 g of NAC in 50 ml of water and exhibited low odor. After several weeks, the solution had evaporated to give concentrated NAC solution with significant crystal formation, and still in this concentrated form the NAC odor appeared greatly reduced. Combinations of EDTA, GLDA, sultaines, betaines, taurates, ectoin, sarcosiates, and the like may result in enhanced odor control, particularly as a function of pH. Of course, using higher-grade NAC such as pharmaceutical grade with low initial odor can also be helpful as a strategy in reducing odor in NAC-containing products. Odor can also be masked to some degree with suitable fragrances or essential oils, but there is a need for more effective means to reduce odor release or formation rather than masking it. In a further test, 8.12 g from stick P4 were removed and melted again, then combined with 0.111 g of sodium hexametaphosphate that had been dissolved in 0.329 ml of water, with 0.83 g cacao butter added to help compensate for the added water content. After thorough mixing, the mass was cooled and kept covered, then its odor compared to the original stick P4, showing good but not complete reduction.

TABLE 2A
Further antiperspirant runs with oil-silicone phases.

| | Run: | | | | |
|---|---|---|---|---|---|
| Oil phase + silicone phase | P5 | P6 | P7 | P8 | P9 |
| Almond oil | | | | 1.691 | |
| Aluminum chlorohydrate | 7.33 | 5.18 | 6.276 | 6.24 | 4.7 |
| AqStar M1 ® | 0.351 | | 0.821 | 0.255 | |
| Arrowroot starch | 5 | 3.54 | 3.5 | 1.62 | 4.03 |
| Bentone Gel GTCC V | | 0.90 | 0.90 | 0.80 | 1.20 |
| C26-28 alkyl dimethicone | 0.33 | | | 0.78 | 1.14 |
| Cacao butter | 2.25 | 1.50 | 1.51 | | 2.62 |
| Caprylyl dimethicone | 1.53 | 0.72 | 0.72 | 0.64 | 0.96 |
| DM6 dimethicone | 3.89 | 3.59 | 3.61 | 3.22 | 4.81 |
| DM350 dimethicone | 1.70 | 1.49 | 1.49 | 1.33 | 1.99 |
| Ethylhexyl palmiate | | | | 1.687 | |
| Gelmaker CC | 1.14 | | | | |
| GLDA | 0.66 | 0.56 | 0.29 | | 1.11 |
| Isoamyl laurate | 3.56 | 4.58 | 4.606 | 1.39 | 4.665 |
| Jojoba oil | 1.065 | 0.28 | 0.28 | 1.32 | |
| Kostol PGP | 0.419 | 0.33 | 0.335 | 0.356 | 0.343 |
| Olivem 1000 | | | | 1.988 | 0.833 |
| Panthenol | 0.279 | 0.198 | 0.224 | | |
| Pea starch | | | | 2.63 | |
| Polymethylsilsesquioxane | 4.00 | 2.345 | 2.795 | 2.19 | 2.84 |
| Sodium cocoyl glutamate | 1.276 | | | | |
| Sodium methyl cocoyl taurate | 1.59 | 3.2 | 3.412 | | |
| Squalane | 0.26 | | | | |
| Stearic acid | | 0.756 | | | |
| Stearyl alcohol | 4.06 | 2.55 | 3.323 | 3.66 | 2.436 |
| Synkos | 4.74 | 5.04 | 5.16 | 5.166 | |
| 2050 wax | | | | | |
| Water phase addition: | 4.57 | 5.00 | 4.16 | 5.17 | 7.51 |
| Water correction (est. evap. loss) | 0.1 | 0.1 | | | |
| Total mass (g): | 49.90 | 41.65 | 43.42 | 42.14 | 41.19 |

Good results, with reasonable stability and good texture of the solid material, were obtained in these runs except for Run P6, where upon addition of the powders at the end (starch, antiperspirant material and polymethylsilsesquioxane), there was large-scale agglomeration and instability, making it impractical to even fill a deodorant barrel. It was speculated that the presence of stearic acid and/or the absence of the cucurbituril emulsifier, AqDot's AqStar M1®, may have contributed to the instability. Run P9 was similar but instead of AqStar M1® had added Olivem 1000® emulsifier and also exhibited excellent stability and good tactile properties

TABLE 2B
Acid paste compositions corresponding to the
“acid paste addition” listed in Table 2A.

| Water Phase | | | | | |
|---|---|---|---|---|---|
| Composition | AP32 | AP32 | AP33 | AP34 | AP35 |
| Used in runs: | P5 | P6 | P7 | P8 | P9 |
| Water | 14.90 | 14.80 | 2.00 | 4.83 | |
| Mandelic Acid | 6.10 | 6.10 | 2.57 | 2.13 | 3.28 |
| NAC | 3.05 | 3.05 | 3.04 | 2.22 | 1.80 |
| Glycerin | | | | | 6.00 |
| Propane(1,3)diol | 8.20 | 8.20 | 8.96 | 2.36 | 2.80 |
| Caffeine | 1.00 | 1.00 | | | |
| NaOH | | | 0.612 | | |
| GLDA | | | | 7.11 | |
| Hyaluronic acid | 0.20 | 0.20 | | | |
| Soluble starch | 0.24 | 0.24 | | | |
| Acrylic copolymer | 0.01 | 0.01 | | | |
| Citric acid | | | 0.508 | | |
| Ectoine | | | 0.157 | | |
| N-acetyl glucosamine | | | | | 0.55 |
| Sodium lauroyl sarcosinate | | | | 2.18 | |
| Sodium cocoyl glutamate | | | | 1.15 | |
| Acrylate copolymer | | | | | 0.02 |
| Cocoamid. Hyd. Sultaine | 2.61 | 2.61 | | 1.17 | |
| Initial mass: | 36.30 | 36.20 | 17.84 | 23.15 | 14.45 |

TABLE 2C
Overall percentage of ingredients in final sticks.

| Summary of Key | | | | | |
|---|---|---|---|---|---|
| Components: | P5 | P6 | P7 | P8 | P9 |
| Aluminum | 14.66% | 12.41% | 14.46% | 14.81% | 11.41% |
| chlorohydrate %: | | | | | |
| Caffeine %: | 0.25% | 0.33% | 0.00% | 0.00% | 0.00% |
| Mandelic acid %: | 1.54% | 2.01% | 1.38% | 1.13% | 4.14% |
| Silicone | 22.9% | 19.5% | 19.8% | 19.4% | 28.5% |
| compounds %: | | | | | |
| Synthetitc wax %: | 9.5% | 12.1% | 11.9% | 12.3% | 0.0% |
| Starch %: | 10.06% | 8.56% | 8.06% | 10.08% | 9.78% |
| Water %: | 3.75% | 4.92% | 1.07% | 2.56% | 0.00% |
| Glycerin %: | 0.00% | 0.00% | 0.00% | 0.00% | 7.57% |
| Esters (with | 14.3% | 15.2% | 14.7% | 14.4% | 17.7% |
| triglycerides) %: | | | | | |
| Fatty alcohols | 8.1% | 7.9% | 7.7% | 8.7% | 5.9% |
| & acids %: | | | | | |
| NAC: | 0.77% | 1.01% | 1.63% | 1.18% | 2.27% |
| Diol/glycol %: | 2.06% | 2.70% | 4.81% | 1.25% | 3.53% |
| NaOH: | 0.00% | 0.00% | 0.33% | 0.00% | 0.00% |
| Gelling agents, | 3.81% | 2.94% | 4.82% | 8.07% | 5.77% |
| emulsifiers: | | | | | |
| Panthenol: | 0.56% | 0.47% | 0.52% | 0.00% | 0.00% |
| GLDA | 1.33% | 1.35% | 0.67% | 3.77% | 2.69% |
| Sultaines, taurates, | 6.39% | 8.53% | 7.86% | 2.38% | 0.69% |
| sarcosinates, | | | | | |
| glutamates, | | | | | |
| and glucosamines | | | | | |
| Other (hyaluronic | 0.05% | 0.07% | 0.27% | 0.00% | 0.03% |
| acid, acrylate | | | | | |
| copolymer, | | | | | |
| citric acid) | | | | | |
| Total: | 100% | 100% | 100% | 100% | 100% |
| pH | 4 | | | 4 | 3.7 |

This approach to acid stick production is built upon inventive work seeking to overcome the basic challenges of producing an acidic solid stick. The related experimental work for that initial phase of developing the inventive product as claimed herein is shown in the examples below, illustrating some of the scope of the novel approach to creating acidic sticks.

In many runs prior to run 100, separate oil and silicone phases were prepared and heated, and after heating to 70° C. to 85° C., depending on the particular mix of compounds, were then combined in a single well in the double boiler system and mixed by hand with a whisk or whisk and spoon, together or in succession. Then the acid paste/water phase mixture was added and blended using a whisk or combination of spoon and whisk, followed by addition of starch and possible other powdered materials such as polydimethylsilsequioxane. At that point final ingredients could be added such as essential oils and/or caprylyl dimethicone, though in later embodiments (after run 106) caprylyl dimethicone was blended into the silicone and oil phase prior to mixing with the water phase. After blending in of the starch and other powdered ingredients and the final ingredients, if any, the hot mixture was immediately poured into a deodorant mold, using various molds such as repurposed commercial deodorant containers, 15 ml oval shaped molds, 2.2 ounce round plastic molds, and clear acrylic cylindrical Juvitus® brand (Culver City, Calif.) 1-ounce molds.

Starting with run 106, all silicone compounds including caprylyl dimethicone, if present, were combined and heated with the oil phase. Starting with run 98, the Bentone gel and dimethicones or other silicone liquids were combined in a large batch, large enough for over 3 runs, and then blended with an immersion mixer before adding to the oil phase and other ingredients, and the mixture was then heated and stirred/whisked together prior to the addition of a heated acid paste, followed by arrowroot starch and polymiethylsilsequioxane powder, when present.

In Tables 3A through 5C below, ingredients blended into the combined oil and silicone phase are shown in Tables 3A, 4A, and 5A, including oils, waxes, and esters including triglicerides, emulsifiers, silicone compounds, and powders that were blended into the mix. The water phase ingredients (also sometimes called the acid paste) are show in Tables 3B, 4B, and 5B, and the amount of the respective water phase/acid paste blended with the oil and silicone phase is listed as the entry for “Water phase add. (addition)” toward the end of each of Tables 3A, 4A, and 5A, which each have slight differences in the collective group of ingredients used. With the combination of the oil and silicone phase, the water phase, and other final ingredients (arrowroot starch, optionally caprylyl dimethicone, optionally polymethylsilsequioxane and fragrances in some early runs), the calculated net composition by ingredient categories is shown in each of Tables 3C, 4C, and 5C. In some cases, as estimate for water loss during mixing is provided which is based on measured mass losses for water during its blending into a hot oil phase, based on rough measurements made as a heated mass was blended over time, using the removable well as an easy-to-weigh container in some experiments, and considering the duration of time at elevated temperature prior to pouring and cooling. The estimated water loss during processing is in Tables 3A, 4A, and 5A below “Water Phase Addition.”

Not all runs are shown, sometimes because they involved peripheral experimental work outside the scope of this disclosure, or occasionally involves experimental mistakes (e.g., adding excessive starch) or other problems. Many early runs focused on simply demonstrating the possibility of making a stable and non-gritty deodorant at all with high mandelic acid content and employed combinations with existing commercial products that often resulted in problems with texture, stability, etc. For example, combinations of the acid paste with existing deodorants high in alkaline materials such as sodium bicarbonate or magnesium hydroxide resulted in frothing, instability, or other setbacks or could not reach desired pH levels without excessive and wasteful additional mandelic acid. Some of these are reported but not all.

TABLE 3A
Series of runs with oil, silicone, and water.

| Run: | 74 | 75 | 76 | 77 | 78 | 79 | 80 | 81 | 83 | 84 | 85 |
|---|---|---|---|---|---|---|---|---|---|---|---|
| Oil + silicone phase | | | | | | | | | | | |
| Arrowroot starch | | | 2.82 | 2.71 | 2.81 | 8.68 | 3 | 5.3 | | 7.38 | 7.31 |
| Beeswax | 1.834 | | | | | | | | | | |
| Behenyl alcohol | | | | 1.281 | | 4.078 | 2.4 | | | | |
| Behenyl CreamMaker | | | | 0.523 | | | | | 0.51 | 0.607 | 0.607 |
| Bentone Gel GTCC V | 3.011 | 2.51 | 3.041 | 2.01 | 0.8 | 2.05 | 2.2 | 2.45 | 1.123 | 1.676 | 1.676 |
| C26-28 alkyl DM | 1.21 | 4.907 | 4.628 | 0.429 | | 1.093 | 0.62 | 3.2 | 1.572 | 2.514 | 2.514 |
| Cacao butter | 0.929 | 4.225 | 7.74 | | | | | | | | |
| Caffeine (oil/sil. phase) | | | | 0.2 | | | 0.2 | 0.2 | 0.2 | 0.2 | 0.1 |
| Candelilla wax | 1.021 | 1.206 | 0.743 | 0.538 | | 0.535 | 1.754 | 0.37 | | 0.505 | 0.505 |
| Caprylic/capric trigly. | | | | 5.128 | | | | | | | |
| Caprylyl dimethicone | | | | | | | | | 0.3 | | |
| Castor wax | | | | | | | | | | 0.723 | 0.73 |
| Cetyl alcohol | | | | 2.17 | 3.321 | 1.623 | | | | | |
| DM-v-DM copolymer | | | | | | | | | | | |
| DM6 dimethicone | | | | 0.241 | 0.376 | 0.25 | | 0.355 | 1.172 | 0.262 | |
| DM350 dimethicone | | 20.683 | 0.37 | 0.638 | 0.652 | 0.232 | 0.917 | 0.986 | 0.67 | 0.495 | 0.495 |
| DM4200 dimethicone | | | | | | | | 0.441 | | | |
| Diphenyl siloxy PTM | | | | 0.205 | 0.372 | 0.353 | 0.54 | 0.603 | 0.154 | 0.27 | 0.27 |
| Ecosil | | | 1.777 | | | 16.21 | 10.9 | 12.84 | | | |
| Ethylhexyl palnniate | | | 1.64 | 0.87 | | 0.223 | 0.669 | | 0.402 | | |
| Glycerin (oil phase) | | | 1.26 | 0.73 | 0.635 | 0.787 | 0.787 | 1.569 | | | |
| Hennisqualane | 2.99 | | | | | | | | | | |
| Hydroxpr.-cocoate | 0.841 | | | | | | | | | | |
| PEG-8 DM | | | | | | | | | | | |
| Isoamyl laurate | | | | | 4.958 | | 2.61 | | 4.26 | 7.07 | 7.08 |
| Kostol PGP | | | | | | 0.26 | 0.403 | 0.505 | 0.105 | 0.176 | 0.176 |
| Lauryl laurate | | | | 4.49 | 2.3 | 7.4 | 3.41 | 4.028 | | | |
| Magnesium stearate | | | | | | | | | 0.492 | | |
| Panthenol | | | | | 0.147 | 0.285 | 0.352 | 0.3 | 0.151 | 0.253 | 0.253 |
| Polynnethylsilsesquiox. | 3.011 | | 1.00 | | | | 2.93 | | | 1.85 | 1.59 |
| Silica dimethyl silylyl | | | | 0.78 | | 1.303 | 0.365 | | | | 0.48 |
| Squalane | | 0.904 | 0.83 | 0.53 | 0.383 | | | 0.586 | 0.809 | 0.399 | 1.048 |
| Stearyl alcohol | | | 1.69 | 1.008 | | 2.778 | 4.068 | 8 | 3.033 | 5.005 | 5.005 |
| Water phase add.: | 9.2 | 3.07 | 2.4 | 2.465 | 2.433 | 3.51 | 3 | 4 | 2.63 | 4.26 | 4.49 |
| Water loss (est.) | 0.2 | 0.1 | | 0.1 | 0.1 | 0.1 | | | | 0.1 | 0.1 |
| Total mass (g): | 23.85 | 37.41 | 29.94 | 26.85 | 19.09 | 51.55 | 41.13 | 45.73 | 17.09 | 34.04 | 34.23 |

Not listed are fragrances in some cases. For example, run 74 had 3 drops (0.076 g) of elemi essential oil added before pouring.

In run 74, the polymethylsilsequioxane powder (3.011 g) was added to a first oil-silicone phase with 1.83 g beeswax, 1.021 g candelilla wax, 0.929 g cacao butter, and 1.21 g C26-28 alkyl dimethicone, while an oil-gel phase was made from 0.841 g of hydroxypropyl-PEG-8 dimethicone, 3.011 g of Bentone gel, 2.999 g hemisqualane, and essential oil. Once heated and blended separately, the two were combined at 80° C. and then 9.2 g of Acid Paste 10 was gradually blended in with a whisk. This occurred in the removable well allowing weighing of the unit before, during, and after the blending process. Acid Paste 10 comprised glycerin as the thickener with about 15% glycerin and about 15% mandelic acid present in the aqueous phase. The final product had 24% water, 6% each of glycerin and mandelic acid, over 21% silicones, etc. The resulting product was too soft and not a viable candidate for a stick, possibly because of too high a water level for a silicone+oil+water+acid+thickener formulation. In this case, in retrospect it is proposed that a water level less than 23%, less than 20%, less than 18%, or less than 16%, 15%, 14%, 13%, 12%, 11%, 10% or 9%, such as from 2% to 20%, 3% to 20%, 4% to 18%, 5% to 23%, 1% to 12%, etc., may have been helpful in providing a more suitable, harder composition.

Several acid pastes were used for runs 75-85, as shown in Table 3B.

TABLE 3B
Water phase compositions used for “water phase” addition
listed in Table 3A for the runs listed.

| Water Phase ID | AP10 | AP10A | AP11 | AP12 |
|---|---|---|---|---|
| Used in runs: | 74 | 75 | 76-81 | 82-85 |
| Composition: | | | | |
| Water | 21.7 | 24.73 | 31 | 10.90 |
| Tapioca Starch | 0 | 0.44 | 1.35 | 0.47 |
| Mandelic Acid | 5.108 | 4.06 | 1.878 | 0.66 |
| NAC | 0 | 0.62 | 1.901 | 0.67 |
| Glycerin | 5.04 | 3.40 | 0 | 2.50 |
| Propane(1,3)diol | 0 | 0 | 0 | 1.10 |
| Propylene glycol | 0 | 0 | 0 | 1.52 |
| Caffeine | 0 | 0 | 0 | 0.00 |
| NaOH | 1.29 | 0.87 | 0.00 | 0.00 |
| Initial mass: | 33.14 | 34.11 | 36.13 | 17.82 |

TABLE 3C
Overall percentage of ingredients in final sticks.

| Run: | 74 | 75 | 76 | 77 | 78 | 79 | 80 | 81 | 83 | 84 | 85 |
|---|---|---|---|---|---|---|---|---|---|---|---|
| Oil + silicone phase | | | | | | | | | | | |
| Caffeine %: | 0.00% | 0.00% | 0.00% | 0.74% | 0.00% | 0.00% | 0.49% | 0.44% | 1.10% | 0.57% | 0.29% |
| Mandelic acid %: | 5.95% | 0.98% | 0.42% | 0.48% | 0.66% | 0.35% | 0.38% | 0.45% | 0.54% | 0.45% | 0.47% |
| Silicone %: | 21.2% | 68.4% | 20.0% | 8.54% | 7.33% | 6.27% | 13.1% | 12.2% | 27.3% | 17.8% | 17.6% |
| Synthetitc wax %: | 0.00% | 0.00% | 0.00% | 0.00% | 0.00% | 0.00% | 0.00% | 0.00% | 0.00% | 0.00% | 0.00% |
| Starch %: | 0.00% | 0.11% | 9.72% | 10.4% | 15.2% | 17.1% | 7.57% | 11.9% | 0.39% | 21.5% | 21.2% |
| Water %: | 24.4% | 5.68% | 6.88% | 7.51% | 10.4% | 5.65% | 6.26% | 7.50% | 8.84% | 7.19% | 7.55% |
| Glycerin %: | 5.87% | 0.82% | 4.21% | 2.72% | 3.33% | 1.53% | 1.91% | 3.43% | 2.03% | 1.72% | 1.80% |
| Esters, triglycerides %: | 28.4% | 16.9% | 42.5% | 43.1% | 40.0% | 47.3% | 47.0% | 39.0% | 30.1% | 25.0% | 26.7% |
| Fatty alcohols, acids %: | 0.00% | 0.00% | 5.64% | 16.6% | 17.4% | 16.5% | 15.7% | 17.5% | 16.7% | 14.4% | 14.3% |
| NAC: | 0.00% | 0.15% | 0.42% | 0.48% | 0.67% | 0.36% | 0.38% | 0.46% | 0.54% | 0.46% | 0.48% |
| Diol/glycol %: | 0.00% | 0.00% | 0.00% | 0.00% | 0.00% | 0.00% | 0.00% | 0.00% | 2.12% | 1.80% | 1.88% |
| NaOH: | 1.50% | 0.21% | 0.00% | 0.00% | 0.00% | 0.00% | 0.00% | 0.00% | 0.00% | 0.00% | 0.00% |
| Gels, emulsifiers: | 12.6% | 6.71% | 10.2% | 9.44% | 4.19% | 4.48% | 6.33% | 6.46% | 9.56% | 7.06% | 7.02% |
| Panthenol: | 0.00% | 0.00% | 0.00% | 0.00% | 0.77% | 0.55% | 0.86% | 0.66% | 0.83% | 0.73% | 0.72% |
| Other (MG + ZN salts): | | | | | | | | | | 1.41% | |
| Total: | 100% | 100% | 100% | 100% | 100% | 100% | 100% | 100% | 100% | 100% | 100% |
| pH | | 3.3 | 3.2 | | | | | | 3.0 | 4.5 | 3 |

Table 4A shows formulations for runs 93-101, with respective acid pastes shown in Table 4B and final stick compositions by category shown in Table 4C.

TABLE 4A
Series of runs with oil, silicone, and water.

| Run: | 93 | 94 | 95 | 96 | 97 | 98 | 99 | 100 | 101 |
|---|---|---|---|---|---|---|---|---|---|
| Oil phase + silicone | | | | | | | | | |
| Arrowroot starch | 2.54 | 3.8 | 2.51 | 4.31 | 3.71 | 3.82 | 3.82 | 3.71 | 3.55 |
| Bentone Gel GTCC V | 0.92 | 1.06 | 1.06 | 1.68 | 1.82 | 1.78 | 1.59 | 1.65 | 1.65 |
| C26-28 alkyl DM | 2.01 | 1.45 | 1.47 | 1.47 | 2.59 | 2.09 | 2.45 | 2.4 | 1.25 |
| Caprylyl dimethicone | 1.4 | 1.3 | 1.76 | 1.76 | 1.74 | 0 | 3.4 | 1.84 | 1.89 |
| DM6 dimethicone | 1.96 | 2.71 | 2.97 | 4.96 | 5.12 | 5.69 | 6.36 | 6.06 | 6.06 |
| DM350 dimethicone | 0.49 | 0.29 | 0.47 | 0.68 | 0.86 | 0.89 | 0.76 | 0.86 | 0.86 |
| Diphenyl siloxy PMT | 0.94 | | | | | | | | |
| Ecosil | 0.35 | | | | | 4.01 | | | |
| Ethylhexyl palnniate | 1.89 | | | | | | | | |
| Isoamyl laurate | 3.01 | 1.97 | 2.18 | 3.85 | 3.49 | | 4.12 | 4.23 | 4.35 |
| Kostol PGP | 0.28 | | 0.24 | 0.32 | 0.40 | 0.43 | 0.42 | 0.46 | 0.46 |
| Panthenol | 0.24 | 0.27 | 0.11 | 0.18 | 0.20 | 0.20 | 0.27 | 0.25 | 0.25 |
| Polyethylene wax | 3.73 | 5.10 | 4.68 | 7.97 | 8.03 | 7.06 | 7.5 | 8 | 8 |
| Polynnethylsilsesquioxane | 2.1 | 2.01 | 2.01 | 2.95 | 3.50 | 3.79 | 3.79 | 3.05 | 3.05 |
| Stearyl alcohol | 1.70 | 1.93 | 1.89 | 3.22 | 3.30 | 3.33 | 2.5 | 2.5 | 2.5 |
| Synkos 2050 wax | | | | | | | | | |
| Water phase add.: | 5.42 | 5.8 | 4.72 | 9.2 | 7.65 | 7.55 | 7.46 | 7.46 | 7.4 |
| Water loss (est.) | 0.2 | 0.2 | 0.2 | 0.5 | 0.4 | 0.4 | 0.4 | 0.3 | 0.3 |
| Total mass (g): | 28.8 | 27.5 | 25.9 | 42.1 | 42.0 | 40.2 | 44.0 | 42.2 | 41.0 |

TABLE 4B
Water phase compositions used for “water phase” addition listed in
Table 4A for the runs listed.

| Water Phase | | | | | | | |
|---|---|---|---|---|---|---|---|
| Composition | AP14 | AP15 | AP16 | AP17 | AP18 | AP18B | AP19 |
| Used in runs: | 93 | 94 | 95 | 96 | 97 | 98 | 99-101 |
| Water | 50 | 22.2 | 19.79 | 20.27 | 12.27 | 11.50 | 19.40 |
| Tapioca Starch | 3 | 0 | 1.53 | 1.41 | 0.90 | 0.90 | 1.44 |
| Mandelic Acid | 7.94 | 8 | 11.93 | 10.00 | 6.35 | 6.35 | 10.00 |
| NAC | 1.28 | 0 | 0.00 | 0.00 | 0.00 | 0.00 | 0.00 |
| Glycerin | 14 | 3.29 | 2.77 | 0.00 | 0.00 | 0.00 | 0.00 |
| Propane(1,3)diol | 2.75 | 0 | 0.00 | 1.62 | 2.42 | 2.42 | 2.53 |
| Propylene glycol | 2.04 | 0.00 | 0.00 | 0.00 | 0.00 | 0.00 | 0.00 |
| Caffeine | 1.2 | 3.2 | 4.09 | 4.00 | 2.54 | 2.54 | 3.80 |
| NaOH | 0 | 0 | 0.66 | 0.33 | 0.21 | 0.21 | 0.25 |
| Initial mass: | 82.21 | 36.69 | 40.77 | 37.63 | 24.68 | 23.91 | 37.42 |

As Acid Paste 19 was used in several runs with reheating prior to each use, additional water evaporated. The content of 19.40 g of water initially was estimated to be reduced, in effect (in terms of the overall original composition), to 19.10 g for runs 100 and 100. The resulting composition by percentage of the resulting sticks are shown in Table 4C:

TABLE 4C
Overall percentage of ingredients in final sticks.

| Run: | 93 | 94 | 95 | 96 | 97 | 98 | 99 | 100 | 101 |
|---|---|---|---|---|---|---|---|---|---|
| Summary of | | | | | | | | | |
| Key Components: | | | | | | | | | |
| Caffeine %: | 0.27% | 1.84% | 1.83% | 2.33% | 1.87% | 1.99% | 1.72% | 1.81% | 1.85% |
| Mandelic acid %: | 1.82% | 4.60% | 5.34% | 5.81% | 4.68% | 4.98% | 4.53% | 4.77% | 4.87% |
| Silicone compounds %: | 30.9% | 28.2% | 33.6% | 28.1% | 32.9% | 30.9% | 38.1% | 33.7% | 32.0% |
| Synthetic wax %: | 13.0% | 18.6% | 18.1% | 18.9% | 19.1% | 17.5% | 17.0% | 19.0% | 19.5% |
| Starch %: | 9.52% | 13.8% | 10.4% | 11.1% | 9.49% | 10.2% | 9.33% | 9.48% | 9.37% |
| Water %: | 10.8% | 12.0% | 8.08% | 10.6% | 8.10% | 8.03% | 7.87% | 8.39% | 8.56% |
| Glycerin %: | 3.21% | 1.89% | 1.24% | 0.00% | 0.00% | 0.00% | 0.00% | 0.00% | 0.00% |
| Esters, triglycerides %: | 18.2% | 7.17% | 8.43% | 9.15% | 8.31% | 9.97% | 9.35% | 10.0% | 10.6% |
| fatty alcohols & acids %: | 5.89% | 7.00% | 7.32% | 7.66% | 7.85% | 8.29% | 5.68% | 5.93% | 6.10% |
| NAC: | 0.29% | 0.00% | 0.00% | 0.00% | 0.00% | 0.00% | 0.00% | 0.00% | 0.00% |
| Diol/glycol %: | 1.10% | 0.00% | 0.00% | 0.94% | 1.78% | 1.90% | 1.15% | 1.21% | 1.23% |
| NaOH: | 0.00% | 0.00% | 0.30% | 0.19% | 0.15% | 0.16% | 0.11% | 0.12% | 0.12% |
| Gels, emulsifiers: | 4.17% | 3.86% | 5.02% | 4.76% | 5.29% | 5.50% | 4.58% | 5.01% | 5.15% |
| Panthenol: | 0.83% | 0.99% | 0.43% | 0.43% | 0.48% | 0.51% | 0.61% | 0.59% | 0.61% |
| Total: | 100% | 100% | 100% | 100% | 100% | 100% | 100% | 100% | 100% |
| PH | 2.5 | 25-2.9 | 2.9 | 2.9 | 3 | 2.5 | 2.8 | 2.7-2.9 | 3.2 |

Run 93 included some N-acetyl cysteine in the acid paste. Without wishing to be bound by theory, it is proposed and believed that the low pH of N-acetyl cysteine coupled with its potential antimicrobial or anti-biofilm capabilities may be compatible with the mechanisms of mandelic acid in enhancing the skin microbiome and thus may be a particularly useful ingredient for a deodorant, although in high concentrations it can provide a sulfurous odor. The 0.3% concentration in this sample did not lead to obvious sulfurous odors and appeared to be compatible with the formulation. Other earlier runs also showed that even higher concentrations of NAC could be successful and gave positive results in testing on human subjects, though the sulfurous smell of NAC was sometimes noted.

Run 101 was repeated but with 3 different pour temperatures, 78° C., 68° C., and 63° C., with substantially the same quantity poured into identical 2.2-ounce round deodorant molds and cooled to about 72° C. Hardness was measured using the AMS 59032 E-280 Pocket Penetrometer. A hardness of 0.6 was recorded for the pour at 78° C., 0.5 for 68° C., and 0.4 for 63° C.

Table 5A shows formulations for runs 102-110, with respective acid pastes shown in Table 5B and final stick compositions by category shown in Table 5C.

TABLE 5A
Series of runs with oil, silicone, and water.

| Run: | 102 | 103 | 104 | 105 | 106 | 108 | 109 | 110 |
|---|---|---|---|---|---|---|---|---|
| Oil phase + silicone | | | | | | | | |
| Arrowroot starch | 3.59 | 3.56 | 3.55 | 3.54 | 3.55 | 3.55 | 3.67 | 3.62 |
| Bentone Gel GTCC V | 1.16 | 1.64 | 1.64 | 1.64 | 1.61 | 1.62 | 1.64 | 1.66 |
| C26-28 alkyl DM | 0.95 | 1.25 | 1.25 | 1.25 | 1.25 | 1.25 | 1.25 | 1.24 |
| Caprylyl dimethicone | 1.89 | 1.89 | 1.89 | 1.84 | 1.932 | 1.88 | 1.96 | 1.94 |
| DM6 dimethicone | 4.25 | 6.00 | 6.00 | 6.01 | 6.05 | 6.10 | 6.17 | 6.27 |
| DM350 dimethicone | 0.60 | 0.86 | 0.86 | 0.86 | 0.86 | 0.86 | 0.88 | 0.90 |
| Isoamyl laurate | 4.3 | 4.35 | 4.38 | 4.38 | 4.37 | 4.37 | 4.36 | 4.54 |
| Kostol PGP | 0.31 | 0.46 | 0.46 | 0.46 | 0.46 | 0.46 | 0 | 0.47 |
| Panthenol | 0.26 | 0.28 | 0.28 | 0.28 | 0.29 | 0.27 | 0.27 | 0.27 |
| Polyethylene wax | 8.03 | 8.04 | 8.03 | | | | | |
| Polynnethylsilsesquiox. | 3.3 | 2.87 | 2.86 | 2.86 | 2.87 | 2.02 | 1.76 | 1.83 |
| Stearyl alcohol | 2.51 | 2.5 | 2.51 | 2.51 | 2.51 | 2.5 | 2.5 | 2.66 |
| Synkos 2050 wax | | | | 8.04 | 8.04 | 8.01 | 8.01 | 8.09 |
| Water phase add.: | 7 | 7.39 | 7.39 | 7.14 | 7 | 7.37 | 7.53 | 6.75 |
| Water loss (est.) | 0.3 | 0.2 | 0.2 | 0.2 | 1 | 0.1 | 0.1 | 0.1 |
| Total mass (g): | 37.8 | 40.9 | 40.9 | 40.6 | 39.8 | 40.2 | 39.9 | 40.1 |

TABLE 5B
Water phase compositions used for “water phase” addition
listed in Table 5A for the runs listed.

| Water Phase | | | | | |
|---|---|---|---|---|---|
| Composition | AP19 | AP20 | AP23 | AP24 | AP25 |
| Used in runs: | 102 | 103 | 108 | 109 | 110 |
| Water | 19 | 20 | 19 | 9.7 | 13 |
| Tapioca Starch | 1.44 | 1.43 | 1.44 | 1.56 | 0.68 |
| Mandelic Acid | 10 | 10 | 10 | 11.11 | 10 |
| Propane(1,3)diol | 2.53 | | 2.54 | 11 | 6.5 |
| Propylene glycol | 0 | 2.54 | | | |
| Caffeine | 3.8 | 3.8 | 3.73 | 3.8 | 3.8 |
| NaOH | 0.25 | 0.325 | 0.345 | 0.347 | |
| Initial mass: | 37.0 | 37.8 | 37.0 | 37.5 | 34.3 |

As Acid Paste 20 was used in several runs with reheating prior to each use, additional water evaporated. The content of 20.0 g of water initially was estimated to be reduced by evaporation, in effect, in terms of the overall original composition, to 19.5 g in Run 104, 19.2 g for run 105, and 18.9 g for run 106. The resulting composition by percentage of the resulting sticks are shown in Table 5C:

TABLE 5C
Overall percentage of ingredients in final sticks.

| Run: | 102 | 103 | 104 | 105 | 106 | 108 | 109 | 110 |
|---|---|---|---|---|---|---|---|---|
| Summary of | | | | | | | | |
| Key Components: | | | | | | | | |
| Caffeine %: | 1.90% | 1.82% | 1.84% | 1.81% | 1.82% | 1.85% | 1.91% | 1.86% |
| Mandelic acid %: | 5.00% | 4.79% | 4.85% | 4.75% | 4.80% | 4.96% | 5.59% | 4.90% |
| Silicone compounds %: | 29.0% | 31.5% | 31.4% | 31.6% | 32.6% | 30.2% | 30.1% | 30.3% |
| Synthetic wax %: | 21.2% | 19.7% | 19.6% | 19.8% | 20.2% | 19.9% | 20.1% | 20.2% |
| Starch %: | 10.2% | 9.39% | 9.37% | 9.39% | 9.61% | 9.55% | 9.99% | 9.35% |
| Water %: | 8.70% | 9.08% | 8.97% | 8.64% | 6.55% | 9.17% | 4.63% | 6.12% |
| Glycerin %: | 0.00% | 0.00% | 0.00% | 0.00% | 0.00% | 0.00% | 0.00% | 0.00% |
| Esters, triglycerides %: | 11.4% | 10.6% | 10.7% | 10.8% | 11.0% | 10.9% | 10.9% | 11.3% |
| Fatty alcohols & acids %: | 6.63% | 6.11% | 6.14% | 6.17% | 6.30% | 6.22% | 6.27% | 6.63% |
| Diol/glycol %: | 1.26% | 1.22% | 1.23% | 1.21% | 1.22% | 1.26% | 5.53% | 3.18% |
| NaOH: | 0.12% | 0.00% | 0.00% | 0.00% | 0.00% | 0.16% | 0.17% | 0.17% |
| Gelling agents, | 3.88% | 5.14% | 5.13% | 5.18% | 5.21% | 5.18% | 4.11% | 5.31% |
| emulsifiers: | | | | | | | | |
| Panthenol: | 0.68% | 0.69% | 0.68% | 0.70% | 0.72% | 0.67% | 0.68% | 0.67% |
| Total: | 100% | 100% | 100% | 100% | 100% | 100% | 100% | 100% |
| pH: | 2.7 | 2.5 | N/A | N/A | 2.5 | 2.7 | 2.5 | 2.5 |

The majority of the runs shown above resulted in sticks that solidified well with a range of textures suitable for a solid stick. Granules of mandelic acid could not be perceived if they were present. Rather, the sticks were smooth and generally seemed highly uniform. A number of products were tested on human volunteers with excellent performance, both in terms of application and non-irritation, but also in terms of odor control performance. Water levels between 3 and 20% or 5 and 15% appeared to be capable of providing surprisingly high concentrations of mandelic acid without the problem of graininess and irritation of the skin. Based on further experimental work in which large quantities of caffeine and mandelic acid were dissolved in various elevated viscosity fluids such as water and tapioca starch, glycerin, propanediol, and propylene glycol, it was observed that these liquids often can be easily saturated with the dissolved solids at elevated temperature (e.g., 70° C., 75° C., 80° C., 85° C., 90° C., 95° C., 100° C., 110° C. and 120° C.) and then, upon cooling to room temperature, while solids can precipitate, the precipitate tends to be very fine and often difficult for human skin to perceive the existence of a solid phase. While mandelic acid dissolved in water alone can readily give large crystals after cooling, the thickened fluids described herein seemed, without wishing to be bound by theory, to help control crystallization to reduce the formation of large grains, though fine feathery, needlelike particles believed to be caffeine crystals could be seen in a microscope. It is also possible that caffeine preferentially precipitated leaving high levels of mandelic acid in the liquid.

Run 56. A new Acid Paste was made using N-acetyl cysteine (NAC) instead of mandelic acid. 50 ml of water were combined with 4.40 g of corn starch and 5.0 g NAC. This was place in a 100-ml beaker and heated by microwave, stirring between brief bursts of power, to finally create a smooth, uniform paste. This is Acid Paste 8.

The oil phase was made from 7.50 g cacao butter, 2.00 g sunflower wax, 0.89 g coconut oil, 0.2 g caffeine, 1.58 g emulsifying wax, 5.03 g caprylic capric triglycerides MCT, 5.56 g stearyl alcohol, 0.66 g palmitic acid, 0.118 g magnesium myristate, and 0.297 g silica dimethyl silylate. This was blended with 2.17 g Acid Paste 8 and then with 3.00 g arrowroot starch, and then with 2 drops of elemi oil. The effective pH was about 3.5.

Run 57. The oil phase was made from 9.57 g cacao butter, 2.17 g candelilla wax, 1.39 g coconut oil, 0.3 g caffeine, 1.89 g emulsifying wax, 7.43 g caprylic capric triglycerides MCT, 5.15 g stearyl alcohol, and 1.8 g ethylhexyl palmitate. To the oil phase was added 2.06 g Acid Paste 8, 3.45 g arrowroot starch, 1 drop elemi oil and 2 drops lavender oil. After cooling, this was too soft, so it was remelted and combined with 2.00 g castor wax, 1.15 g behenyl alcohol, and 1.35 g tapioca starch.

Dimethicone Series: Three dimethicones were used with viscosities of 4200-4800 cst (DM4200), 350 cst (DM350), and 6 cst (DM6).

### Examples of Vitamin C Serums

7.39 g of 99.93% ascorbic acid powder (Resurrection Beauty, Holmen, Wis.) was combined with 0.45 g panthenol, 1.28 g NAC, 45 ml of water, 2.20 g of lactic 1.2 g NaHCO₃, showing a pH of 3.4. Of this, 39 g was put in a spray bottle and labeled as Serum A. 16.0 g was put into another bottle to which 0.1 g caffeine was added and 0.15 g hyaluronic acid (SLMW). This was Serum A1. Both products were used on the faces of two subjects for testing over a period of several weeks, with one subject reporting noticeable improvement relative to facial wrinkles. However, the application sometimes caused mild stinging or a noticeable film forming effect. An improved formulation was requested that would be gentler or smoother.

A new formulation was made with Phase A comprising 67.8 g water, 0.963 g NAC, 0.754 g panthenol, and 0.394 g sodium carbonate. The mixture was placed in a wide dish and 0.158 g hyaluronic acid (SLMW) were sprinkled onto the surface and allowed to slowly hydrate. Phase B was prepared with 20 g ethoxydiglycol, 14.9 g propanediol, 2.40 g glycerin, 0.77 g of Vitamin E oil from Spring Valley (Walmart, Bentonville, Ark.) 400 IU capsules, 0.44 g phenylpropanol, and 4.00 g laureth-23 from Lotioncrafter LLC. After Phase B was heated to about 60° C. and blended, it was stirred into Phase A. The appearance immediately became cloudy. 21 g of water and 0.95 g coco betaine were added to see if the cloudiness could be reduced, without success, but the creamy appearance, due to phenylpropanol, is not necessarily a negative. Then 6.03 g of buffered ascorbic acid from Pure Encapsulations (Sudbury, Mass.) was added with 9.6. g of ascorbic acid powder from Resurrection Beauty and further adjustments to pH, namely, 1.99 g citric acid, 16 g water, 1.74 g NAC, giving a pH of about 3. This was labeled Serum B1. It was noted that the odor of the mixture is very faint, raising the possibility that the betaine was assisting in odor control.

The odor control effect of coco betaine was tested by mixing 2 g of NACA in 60 g of water and dividing the solution evenly between two jars. One jar was given 1.5 g of added water while the other was given 1.5 g added coco betaine. Both were allowed to sit covered for a period of time and then tested for odor. The jar with the added betaine had distinctly less odor. Over an hour later, some sulfur odor was detected, so 0.5 g of cocoamidopropyl hydroxysultaine was also added, but this did not significantly improve odor control. (This was before noting the effect of SHMP.)

Serum C was made with 73.6 g water, 1.71 g NAC, and 0.326 g hyaluronic acid (SLMW) in phase A, with phase B having 1.15 g panthenol, 14.91 g ethoxydiglycol, 3.67 g propylene diglycol, 1.42 g cocoa betaine, 6.95 g propane diol, 0.62 g phenylpronol, 0.35 g Vitamin E oil, 0.08 g of HandSan Clean Lemon scent of Air-Scent International (Pittsburgh, Pa.), and 10.87 g ascorbic acid powder. After the two phases were blended, 0.68 g NaHCO₃ was added, giving a pH of 2.8.

Serum D was made with 81.9 g water, 0.88 g NAC and 0.25 g hyaluronic acid (SLMW) in Phase A. Phase B had 24.0 g ethyoxydiglycol, 0.9 g panthenol powder, 3.91 g propylene glycol, 2.8 g propane diol, 2.60 g laureth-23, 0.40 g Leucidal SF Max preservative, 0.04 g orange essential oil, 0.03 g frankincense essential oil, 9.79 g ascorbic acid powder, 1.34 g buffered ascorbic acid (Pure Encapsulations), with a pH of 3.26. Batch D was tested on the faces of two subjects daily for over one month, with positive results suggesting increased skin smoothness and apparent decrease in fine wrinkles. The serum seemed to absorb into skin faster than expected, meaning noticeably faster than water alone.

Serum D2 was made from 4.76 g of Serum D, with 0.44 g almond oil added, 0.15 g jojoba oil, and 0.29 g coco betaine, blended thoroughly. This creamy preparation felt smooth and comfortable when applied to the skin, like a lotion.

Serum E was made from 58.3 g water, 1.15 g NAC, 0.316 g hyaluronic acid (SLMW) in Phase A. Phase B had 0.31 g Vitamin E oil, 11.38 g propanediol, 2.14 g glycerin, 5.96 g ethoxydiglycol, 0.379 g panthenol, 0.57 g jojoba oil, 2.04 g cocobetaine, 1.20 g laureth-23, 0.046 g HandSan Clear Lemon scent, 0.20 g Leucidal preservative, 1.57 g coffee seed extract (Making Cosmetics, Inc.). After blending the two phases, 0.46 g lactic acid and 0.183 g phenyl propanol were added (the latter largely dissolved rather than forming a milky colloid). Then 8.93 g ascorbic acid powder and 1.303 g of buffered ascorbic acid powder were added, giving a pH of about 3.0. This serum had a pleasant tan color and a good skin feel. This was tested on a subject over a course of one week with a positive response in terms of skin feel and appearance.

The above formulations appear to have better tactile properties in use than the initial Serum A or A1, though all may be suitable, depending on the user.

A second batch of serums was made. For serum Z1, Phase A comprised 78.8 g of water and 0.607 g of hyaluronic acid (SLMW) gently sprinkled onto the surface of the water. After more than 30 minutes, the hyaluronic acid was fully dissolved and the mixture was stirred. Then 0.822 g panthenol was added in 10.8 g water with 1.241 g NAC, 1.120 g SHMP, and 0.21 g sodium carbonate and blended as Phase A. Phase B comprised 3.31 g of Olivem 1000, 19.3 g ethoxydiglycol, 4.00 g propanediol, 1.42 g Luicidal preservative, 4.61 g propylene glycol, 2.2 g glycerin, 0.15 g Vitamin E, 9 g PuriShield CSA solution. Phases A and B were mixed and combined with 10.53 g of buffered Vitamin C powder. The pH was 3.6, which was reduced to 3.3 by adding a small amount of lactic acid.

Serum Z2: Phase A had 63 g water, 1.336 g NAC, 0.851 g panthenol, 1.15 g SHMP, and 0.57 g hyaluronic acid (SLMW). Phase B had 1.126 g Olivem 1000, 1.23 g coco betaine, 7.94 g ethoxydiglycol, 5.69 g propanediol, 0.86 g phenylpropanol. Phases A and B were heated to about 50° C. and blended. The 9.83 g of pure Vitamin C powder was added plus 4.28 g of buffered Vitamin C powder. Some precipitate formed, possibly due to a reaction with the betaine or hyaluronic acid. The pH was 2.8. The pH was raised to 3.3 with 0.11 g sodium carbonate, and 76.7 g of decanted liquid was separated from the precipitate and some froth.

Serum Z3: Phase A had 74.6 g of water, 0.139 g ectoin, 1.585 g NAC, 0.423 g panthenol, 0.678 g trisodium phosphate, 22.89 g PuriShield CSA solution, 0.249 g sodium carbonate, and 0.558 g hyaluronic acid (SLMW). Phase B had 6.22 g aminosyl SCG, 0.93 g laureth-23, 0.17 g Vitamin E, 11.54 g propanediol, 6.00 g ethoxydiglycol, and 0.994 g phenylpropanol. Phases A and B were mixed and had a pH of 6.4 in a clear solution. Then 14.7 g of pure Vitamin C powder was added, giving a pH of 3.2. This serum was applied daily for about 2 weeks to two subjects who reported good skin feel and a penetration into the skin that appeared to be significantly faster than water alone, and without leaving a tacky residue, suggesting that the Vitamin C was penetrating into the skin. Without wishing to be bound by theory, it is believed that the NAC and panthenol in combination may be effective in increasing skin penetration, and thus may assist entry of Vitamin C into the skin, though further investigation is needed to better understand the effect.

Many other ingredients can be considered for serums and other skin care compounds. Water may often be the primary ingredient, followed by 5% to 30% polyol compounds such as one or more of the group comprising propanediol or other diols, glycerine, ethoxydiglycol, propylene glycol or other glycols, etc.; 1% to 25% acids or derivatives thereof such as one or more of the group comprising ascorbic acid, gluconic acids, gluconolactone, lactic acid, mandelic acid, glycolic acid, succinic acid, etc.; optionally 0.5% to 10% of one or more emollients such as butters, oils, esters, lipids, and fatty acids; NAC, typically from 0.1% to 3% or from 0.1% to 1.5%, or from 0.5% to 2%; optionally 0.5% to 8% or 0.5% to 4% of skin permeability enhancers such as panthenol or derivatives thereof, *****; 0.05% to 2% anti-wrinkle agents such as hyaluronic acid or sodium hyaluronate or other salts, particularly the low-molecular weight compounds with a molecular weight of from 1,500 to 150,000, 1,500 to 100,000, 2,000 to 60,000, and 2,000 to 30,000; 0.01% to 5% optional silicone compounds such as dimethicone; suitable emulsifiers when emollients and/or silicone compounds are present and emulsification is needed; optionally 0.1% to 4% or 0.2% to 3% of botanicals, plant extracts, and/or essential oils; optional biofilm control agents; optional preservatives; optional fragrance, etc.

Natural skin care agents such as botanicals, plant extracts, etc., may include Tremella fuciformis extract, Camellia sinensis leaf extract, Rubus idaeus (raspberry) extract, Cupressus sempervirens extract, melon extracts. Salvia officinalis leaf extract, etc.

### Oral Care Examples

Mouthwash M1 was made by combining 53.7 g of Goodsense® Antiseptic Mouth Rise (Geiss, Destin, and Dunn, Inc., Peachtree City, Ga.), “blue mint” flavor, with 0.64 g NAC, 0.91 x xylitol, 4.5 g water, and 0.47 h NaHCO₃. The original pH of 4.3 was now 6.0.

Mouthwash M2 was made by combining 47.5 g of aloe vera juice (filtered whole lead juice, preservative free) from Lily of the Desert (Denton, Tex.) with 2.55 g NAC, 0.10 g peppermint oil, 178 g water, 7.28 g xylitol, 5.67 g sorbitol, 4.91 g propanediol, 2.27 g glycerine, and 1.43 g NaHCO₃, having a pH of 4.6.

M3 was made from 4.74 g xylitol, 3.8 g NAC, 9.64 g sorbitol, 61.9 g aleo vera juice, 210 g water, 10.35 g propanediol, 8.13 g glycerin, and 0.1 g sweet orange essential oil (Healing Solutions, LLC, Phoenix, Ariz.),

M4 was made from 5.74 g sorbitol, 8.42 g xylitol, 1.89 g SHMP (sodium hexmetaphosphate), 2.08 g NAC, 67.1 g aloe vera juice, 0.2 g Vitamin E oil from a capsule, 11.1 g glycerin, 11.1 g glycerin, 5.2 g propanediol, 2.07 g coco betaine, 227.9 g water, and 1.29 g NaHCO₃ to give a pH of 4.9.

M5 was made by taking 40.2 g of Schmidt's® Wondermint mouthwash, comprising water, glycerin, xylitol, blue magnolia bark extract, goji berry extract, etc., and combining it with 50 g of mouthwash M4.

M6 was made from 104 g water, 3.3. g NAC, 0.899 g sodium citrate, 1.38 g SHMP, 1.21 g NaHCO₃, 2.03 g propanediol, 4.19 g xylitol, 32.62 g aloe vera juice and 1.2 g glycerin.

M7 was made from 50 ml of M2 with 1.9 g added NAC and saturated with zinc citrate (2.0 g was added, stirred, and then eventually the solute was decanted from the undissolved solids). Then 38 g aloe vera juice was added to the solute with 1.3 g NaHCO₃ to give a pH of 5.04.

M8 was made with 0.17 g ECGC (98% pure epigallocatechin gallate extracted from green tea), 1.13 g NAC, 42 g water, 0.47 g sodium citrate, 11.9 g aloe vera juice, 1 drop sweet orange essential oil, 3.46 g propane diol, 1.9 g xylitol, and 0.77 g sorbitol, 1.21 g glycerin. After adding 0.58 g NaHCO₃ to raise the pH to slightly over 6, a light pink color developed in the solution, believed to be due to EGCG dimerization. The taste also was somewhat bitter. Then 0.52 g NAC was further added to bring the pH down to 5.2, causing the pink color to fade away. The slight tartness seemed to also improve the taste.

Samples M1 through M8 were sealed in jars overnight and then each individually opened and evaluated by two testers checking for malodor from NAC. Both testers independently rated sample M4, a sample containing SHMP, as being nearly free of malodor. M3 was also favorably rated for the light orange smell seemed to mask NAC odor well. M7 also had only faint NAC odor. The strong mint of M1 and M5 covered much of the NAC odor, but the combination was not completely effective.

The mouthwash solutions were tasted after being stored overnight at room temperature. M1 had a strong mint flavor. M2 has a pleasant citrus flavor. M3 had a citrus flavor similar to grapefruit. M4 was unpleasant. M6 had a salty taste and some NAC flavor that could be detected. M7 had a neutral taste with no distinct flavor. M8 had an unpleasant taste and had developed a slightly purple tinge.

A 3% NAC solution in water was prepared and adjusted with NaOH to a pH of 4.05 and used to make several mixes to test odor control. In mix Z1, 0.533 g of EDTA was added to 50 ml of the 3% NAC solution. In mix Z2, 0.725 g of TSGD (tetrasodium glutamate diacetate) was added to 50 ml of 3% NAC solution. In mix Z3, 0.49 g of TSGD was combined with 50 ml of 3% NAC solution. In mix Z4, 0.583 g of Aminosyl SGC were combined with 50 ml of 3% NAC solution. After being sealed in a 200 ml vessel overnight and then opened and smelled, Z1 was rated at 1 on a scale of 0 to 5 for malodor, being nearly odor free, and Z2 was also rated favorably at 2. Z3 and Z4 had more apparent malodor, being rated at 4 and 5, respectively. Thus sodium cocoyl glutamate and EDTA may be relatively effective in reducing NAC malodor under the conditions studies. A higher amount of TSGD may have contributed to a slightly lower odor level, but the effect was not as strong as observed with some of the other compounds tested.

At this point, deodorant stick P4 containing NAC was opened after being stored for about 10 months. A distinct sulfurous odor was present. 8.12 g of the stick was combined with 0.83 g of cacao butter, 0.321 g water, and 0.111 g SHMP. These were heated in a double boiler and melted together, then mixed and cooled, and stored in a plastic bag. When smelled several hours later, the sulfurous odor was clearly reduced, again suggestive of the power of SHMP in reducing NAC odor.

### Creams, Ointments, Lotions, etc.

Several examples of creams, ointments, lotions, and other consumer products were also made with NAC and related compounds. For example, about 0.3 gram of NAC powder was blended into 5 ml of a diabetic skin care cream, Goicoechea® DiabetTX® Skin Lotion, manufactured by Genomma Lab USA (Houston, Tex.), with listed ingredients including, in order: Water, Glycine Soja (Soybean) Oil, Cetearyl Alcohol, Ceteareth-20, Imidazolidinyl Urea, Peroxidized Corn Oil, Hydrogenated Butylene/Ethylene/Styrene Copolymer, Propylene Glycol, Isopropyl Palmitate, Cyclomethicone, Dimethicone, Sodium PCA, Colloidal Oatmeal, Carbomer, Tocopheryl Acetate, Laminaria (Algae) Extract, Decolorized Aloe Barbadensis Leaf Juice, Mentha Piperita (Peppermint) Oil, Eucalyptus Globulus Leaf Extract, Hydrolyzed Opuntia Ficus Indica Flower Extract, Styrax Benzoin Resin Extract, Myristyl Myristate, Sodium Lactate, Menthol, Sodium Hydroxide, Methylchloroisothiazolinone, Methylisothiazolinone. The NAC dissolved readily into the cream without destabilizing the emulsion. It was then applied onto the skin of two subjects, one on a portion of wound from skin abrasion and also on healthy skin. In both cases there were no adverse effects and it appeared to have softening effect.

In another trial, an aqueous phase was prepared using 0.47 g NAC, 1.71 g propane diol, 0.60 g GLDA, and 1.28 g water. This was blended to dissolve the solids. Then 8.62 g of Eucerin® Advance Repair (a product of Beiersdorf, Hamburg, Germany) was put into a beaker and blended with 2.38 g of sodium methyl cocoyl taurate, and then 0.83 g of the aqueous phase was stirred in. The resulting mixture was less viscous than Eucerin® lotion alone but still appeared stable. It spread smoothly onto skin and had a pleasant but slightly greasy feel, perhaps because of the tactile properties of the taurate compound and the added propanediol. The ingredients of the Eucerin® Advanced Repair lotion are: water, glycerin, urea, cetearyl alcohol, glyceryl glucoside, cyclomethicone, sodium lactate, Butyrospermum Parkii (shea) butter, caprylic-capric-triglyceride, methylpropanediol, octyldodecanol, dicaprylyl ether, tapioca starch, glyceryl stearate SE, hydrogenated coco-glycerides, arginine HCl, sodium PCA, dimethiconol, lactic acid, Chondrus Crispus (carrageenan), carnitine ceramide NP, mannitol, serine, sucrose, citrulline, glycogen, histidine, alanine, threonine, glutamic acid, lysine, sodium chloride, sodium cetearyl sulfate, 1-2-hexanediol, phenoxyethanol.

Creams, pastes, and related agents can be made that combine NAC and related compounds with clays, such as any of the JARXOTIC® clays produced by Jarchem Industries, Newark, N.J. As one example, the formulation for a clay cleansing cream from Elementis (formulation F-027-02) at https://www.ulprospector.com/documents/1592071.pdf?bs=2561&b=1312796&st=20 &r=na&ind=personalcare was adapted for use with NAC. A thick, pleasant clay-based cream with an olive green color was made comprising NAC and related compounds, which had good stability, a smooth emollient-like feel, and good viscosity. When applied to facial skin, it appeared to have a firming effect and could remain for a prolonged time and still feel comfortable. Upon washing, there was no indication of any adverse effects.

The clay cleansing cream was made with three phases. Phase A had 18.23 g of glycerin combined with 7.2 g of Jarxotic® GC-NS, CAS #12173-60-3. Phase B was the aqueous phase with 35 g distilled water, 1.613 g NAC (a relatively low-odor version from Bulk Supplements (Henderson, Nev.), 0.791 g L-cysteine HCL monohydrate from Bulk Supplements, 0.395 g N-acetyl glucosamine from Bulk Supplements, 0.090 g L-methionine from Bulk Supplements, and 0.684 g NaOH. This was stirred and heated to accelerate dissolution, showing a pH of 7.5. This was brought to a temperature of 70° C. and blended with Phase A, and the mixture was brought to a temperature of about 75° C. Meanwhile the oil phase, Phase B, was prepared and melted together to a temperature of about 65° C. It comprises 7.00 g caprylic capric triglycerides MCT, 2.01 g Ecomulse emulsifier, 0.776 g Olivem 1000® emulsifier, 0.995 g cetyl alcohol, 1.270 g stearyl alcohol, 0.987 g Shea butter, and 2.00 g sunflower wax. Phase C was then poured into a heated (about 90° C.) 32-ounce glass Mason jar and the mixture of Phases A and B was also poured in, and a KitchenAid® (St. Joseph, Mich.) immersion blender, Model KHB1231ER with an immersion head that was heated in 90° C. water was then used at high speed (setting #2) to fully blend the mixture as it cooled gradually from about 75° C. to 45° C. At about 43° C., it was transferred from the jar to a broad open glass container and instead of filling the container like a liquid, it acted like a typical thick hand cream and was able to form a mound over a portion of the flat glass bottom of the container, covering an area of about 3 inches by 2 inches with a height of about 1.5 inches.

Without wishing to be bound by theory, it is believed that by elevating the pH of the solution with NAC, initially at a pH of about 1.9, the emulsifiers were able to function effectively in spite of the NAC and related compounds being present. In alternate embodiments, it is believed that the pH could be kept as low as 1.9, 2.0, 2.5, 3, 3.5, or 4 and still maintain good rheological properties and stability if suitable emulsifiers were used, including, for example, AqDot's AqStar M1® emulsifier at a level of 0.2% to 2%.

The use of NAC and related compounds is also envisioned in many other related creams, lotions, pastes, ointments, gels, salves, balms, lipsticks, etc. For example, NAC and related compounds may be effective agents in treating onychomycosis (nail fungus), particularly when combined with keratolyic agents (agents that soften keratin and may help remove outer layers of the skin) that can increase penetration of active agents through the nail. Such agents include urea, salicylic acid, lactic acid, allantoin, glycolic acid, and trichloroacetic acid. As a demonstration of a NAC compound treating nail fungus, a foot soak was prepared in which about 6 g of NAC was combined with about 20 g of urea and combined with a mixture of about 2 liters of hot water and 300 ml of distilled vinegar (5% acidity), wherein two feet from a person with onychomycosis were soaked for about 1 hour, a process that repeated several times, followed by application of various topical ointments. Another water soak was made with 8.75 g NAC, 18.9 g urea, 18.3 g Epsom salt, 3.2 g zinc citrate, 0.6 g trisodiumphosphate, 2.88 g SHMP, 2 g of liquid soap, 1 quart of distilled vinegar (5% acidifty), and 1 gallon of hot water. This mix was then used to soak two feet for 1 hour.

The water soak appears to reduce symptoms and is believed to be synergistic with the topical treatments, but further testing is needed to confirm. It is believed that the urea assists in improving toenail permeability and that the NAC assists in weakening a fungal biofilm so that other agents may be more effective in fighting onychomycosis. It is believed that a further ointment or cream comprising 0.5% to 3% NAC, 1% to 20% urea, and a known antifungal active such as tineacide and/or undecylenic acid, for example, a pH of 2.5 to 9.5 or from 3 to 8 or from 3 to 6, could be helpful in further controlling onychomycosis when applied periodically and topically. Such actives include undecylic acid (typically 25%, but lower levels may also be used), Tolnaftate (typically 1%), Miconazole nitrate (typically 2%), ciclopirox, Tavaborole (Kerydin), Efinaconazole (Jublia), ME 1111, Auriclosene, Luliconazole, camphor oil, application of Vick's Vapo Rub (high in camphor), various essential oils such as rosemary oil, teal oil, grapefruit oil, and/or oregano oil, etc.

In an exemplary trial, 1.13 g of Tineacide® Antifungal Cream from Blaine Labs (Santa Fe Springs, Calif.) was combined with 0.141 g urea, 0.162 g NAC, and 0.04 g N-acetyl D-glucosamine. The mixture was blended with a finger in a small weighing cup for about 5 minutes to dissolve the particles. The viscosity of the cream remained relatively high, though perhaps slightly lower than originally. It is believed that the added urea assisted in controlling the pH and stability of the emulsion.

This was applied to toes both before and after a toe soak with NAC and related ingredients. A powder mix was prepared comprising 10.5 g NAC, 2.50 g N-acetyl D-glucosamine, and 21 g urea. This was dissolved in one quart of warm water and 400 ml of Heinz® distilled vinegar, 5% acidity, and then place in a flat about 25 by 40 cm by 15 cm high. The back of the container was propped up to be about 4 cm off the ground, putting the bottom of the container an angle that pooled the water to fully immerse the toes at the front end of the container. After a soak lasting about 45 minutes and patting the feet to be relatively dry with a cloth, the NAC-laden topical ointment was then applied to the toes, using about 50% of the available amount.

Various personal care and health care products are likewise envisioned in which NAC or related compounds, optionally (when suitable) with acidic ingredients such as mandelic acid, may provide benefits in wound care, treatment of skin ailments such as acne, eczema, psoriasis, dermatitis, allergic reactions, injury from insect bites, relief from itching, healing of burns (particularly in combination with olive oil or olive oil extracts and optionally aloe vera extract in a cream, lotion, or spray), treatment of many wounds, prevention and/or treatment of diabetic ulcers or bed sore, scaliness, excessive keratin growth, prevention of scar tissue (especially when combined with other extracts of onion or garlic known to assist in scar reduction), etc. In such compounds, NAC and related compounds may be present at a level of 0.3% to 12%, for example, and may be dissolved in an aqueous phase at a suitable pH which is combined with an oil phase to form an emulsion (e.g., an 0/W or W/O emulsion) or a solid stick, thus giving a cosmetic, personal care, or health care product that can be conveniently applied, has good texture, and has good stability.

In a variety of tests of 1% to 4% NAC solution combined with proteins such as amylase, pectinase, papain, etc., we found that NAC has a potent effect in reducing microbial growth. Solutions that otherwise would show evidence of significant bacterial growth after days or weeks at room temperature would remain clear and apparently largely untouched by microbes when NAC was present, such that even after 6 months at room temperature such solutions could remain clear, even when the pH had been adjusted to be neutral (e.g., around 6.5 to 8). It is thus proposed that NAC may greatly reduce the need for preservatives in the creams, lotions, sticks, and related cosmetic products described herein. Thus, in one embodiment, NAC-laden cosmetics are proposed that are substantially free of preservatives or that have less than half or less than one-third the amount of preservative otherwise needed to meet common criteria for suppression of microbes such as bacteria and yeast. The PANNAC and PANNAC2 solutions remained clear and apparently free of bacteria for many months after being made, for example.

In another example, the role of NAC as an additive to protective gloves was considered. For example, some gloves have a powder coating on the inside to increase comfort or ease of use. One such powder coating is found in Medline's Restore® Nitrile Exam Gloves with Colloidal Oatmeal, comprising ground Avena sativa flour, paraffin, sodium benzoate, sodium dodecylbenzenesulfonate, sulphur, titanium dioxide, zinc di-nbutyldithiocarbamate, and zinc oxide. It is proposed that reduced antimicrobial agents could be used by taking advantage of NAC's antimicrobial properties, with further skin care benefits. Coupled with paraffin or other waxes and a small quantity of NAC-odor control agents such as polyphosphate ions, NAC may be an effective additive for exam gloves. To demonstrate, 1.65 g of oat flour from Bob's Red Mill Natural Products (Milwaukie, Oreg.) was combined with 0.17 g NAC, 0.124 g SHMP, 0.53 g zinc stearate, and 0.365 g sunflower wax and ground to a fine powder using a mortar and pestle. The powder was then filtered through a fine mesh screen, and 0.11 g of the powder was placed inside a powder-free nitrile exam glove from AMMEX Corp. (Kent, Wash.), “professional series,” and shaken, allowing excess powder to be removed. The glove went on and off more smoothly than an untreated glove and felt more comfortable. There was no noticeable NAC odor in the glove or on the hand after wearing the glove.

NAC added to hand sanitizer such as aqueous ethanol solutions or gels comprising ethanol or other antimicrobials can also be considered, especially in settings where biofilms on solid surfaces are a likely problem. NAC levels may be from, for example, 0.1% to 3% by weight or from 0.1% to 1%. Small amounts of NAC remaining on skin after use of such sanitizers may not only have a humectant and protective effect on the skin, but may assist in weakening biofilms that may be contacted. In such formulations, it may be desirable to add NAC-odor control materials such as SHMP, betaine, quaternary ammonium compounds particularly as antimicrobials, betaine compounds as surfactants or humectants, hydroxysultaine compounds as cleaning agents and moisturizers, etc., each often in the range of 0.3% to 5% or 0.3% to 3%. NAC can serve to protect skin, fight microbes, and undermine biofilm, while the NAC-odor control agent(s) can improve aroma while also providing secondary benefits in terms of skin health, cleaning efficacy, etc.

Lotion Q1: Phase A comprised 5.00 g water, 1.06 g NAC, 0.33 g panthenol, 0.095 g ectoin, 0.12 g allantoin, 0.94 g PuriShield CSA solution, and 0.08 g sodium carbonate. Phase B comprised 3.465 g aminosyl SCG, 0.76 g propanediol, 0.09 Olivem 1000, 0.895 g behenyl alcohol. The two phases were heated and blended, then homogenized with a rotary homogenizer at 13,000 rpm for 60 seconds to form a light lotion. The pH was 2.7.

Cream T5 as a toenail treatment: 0.719 g NAC, 0.285 g ethoxy diglycol, 0.50 g propanediol, 0.791 g PuriShield CSA solution, 0.18 g 25% undecylenic acid in isopropyl palmitate, 0.333 g behenyl alcohol, 0.126 Olivem 1000, 2.38 g Tineacide® antifungal cream (Blaine Labs, Santa Fe Springs, Calif.). The ingredients were blended to yield a relatively thick lotion for treatment of fungus infections in nails. The products was applied to toenails daily for one week with no apparent adverse effects. It is proposed that the antifungal properties of the CSA solution, the Tineacide® cream, and the undecylenic acid combined with the NAC may have the potential to provide synergy with an enhanced antifungal effect, although the permeability of nails can be a challenge. In a related embodiment, urea can be added to soften nails and potentially increase the permeation of active ingredients.

A shaving cream formulation was provided by combining 3.73 g of 2% NAC solution in water with 0.58 g propylene glycol and 3.1 g Barbasol® shaving cream.

An antimicrobial cream was prepared by combining 0.43 g of Nystatin® Cream (100,000 units per gram, from Taro Pharmaceuticals, Hawthorne, N.Y.) with 0.25 g of a 7% NAC solution and blended into a cream less viscous than the Nystatin Cream but still useful and apparently stable. Nystatin® Cream comprises aluminum hydroxide gel, ceteareth-15, glyceryl monostearate, polyethylene glycol 400 monostearate, propylene glycol, purified water, simethicone emulsion, sorbitol solution, TiO2, white petrolatum, methylparaben, propylparaben, and NaOH. This was tested by applying to toenails and skin with no adverse reaction.

### Hair Care

To reduce the effects of oxidation that can lead to the formation of compounds that induce inflammation on the scalp, NAC can be combined with several other ingredients to create hair care products for improved scalp health and reduced inflammation. In some aspects, polyglyceryl-10 laurate at a concentration of 0.1% to 20%, such as from 0.5% to 3%, can be combined with NAC solution and suitable ingredients such as surfactants, emollients, thickeners, etc., to create shampoos, dry shampoos, hair conditioners, and other hair aids, wherein the NAC in the hair product has a concentration of from 0.1% to 5% such as from 0.1% to 2% or from 0.2% to 1.4%, the pH ranges from 3 to 9 such as from 3.5 to 7.5 or from 4 to 7. NAC-odor control agents such as sodium phosphate compounds may be present. The resulting hair products can not only be effective in removing compounds that may contribute to inflammation when oxidized, but may also help prevent oxidation to reduce possible sources of inflammation.

An exemplary dry shampoo, for example, may comprise 0.3-2 parts of a phosphate salt, 0.5 to 1.5 parts NAC, 2 to 60 parts of a surfactant comprising at least 10%, 20%, 30%, 40%, or 50% polyglyceryl-10 laurate or other suitable surfactants, and 10 to 40 parts water. An exemplary serum or anti-dandruff shampoo for the scalp may adapt the formulations given in Pascal Yvon, “Scalp Care 101,” Cosmetics and Toiletries, vol. 136, no. 2 (February 2021): 26, which provides a serum in Formula 1 and a shampoo in Formula 2. Adapting these formulas may be done by adding 0.5 to 1.5% NAC, such as in place of all or part of the citric acid, and adjusting to a suitable pH as needed, and optionally adding 0.1% to 1% panthenol and optionally adding 0.1% to 1% of a diol such as 1-3 propanediol. With the NAC, the serum and the anti-dandruff shampoo may be especially effective in reducing dandruff or itching of the scalp associated with microbial biofilms, and the panthenol in combination with the NAC may help enhance the anti-biofilm effect as well as the comfort provided by the serum and the overall efficacy of the serum. If odor control is desired, both products may incorporate 0.2% to 1% of SHMP or other phosphate salt, or other NAC odor control agents described herein, such as from 0.2% to 2% of the total mass.

### Leather Permeation Tests

Given that the NAC-containing Vit. C serums appeared to become dry on the skin faster than expected compared with water alone, further testing was conducted. Six solutions were prepared as follows: A) 42 g water and 6 g pure Vitamin C powder. B) 42 g water, 5.5 g pure Vitamin C powder, and 0.6 g NAC powder. C) 24.8 g of solution A plus 042 g NAC and 0.200 g panthenol. D) 10.3 g of solution C plus 0.49 g panthenol. E) 19.7 g of solution B plus 0.28 g allantoin, 7.00 g of added water, and 1.80 g of added pure Vitamin C powder. F) 21.0 g og water, 2.73 g of Vitamin C powder, 0.311 g NAC powder, 0.117 g panthenol, 0.115 g allantoin, 0.26 g propanediol, and 0.11 g ethoxydiglycol. Two subjects compared the time required for application of 0.09 to 0.1 g of solution A and B, respectively to become dry after application to the skin, and both concurred that solution B was faster, though they contain similar amounts of solids. A similar test with one subject comparing solution F to A indicated faster absorption by solution F. However, for more clarity and less risk of subjective evaluation, simulations with leather products were conducted. Two types of leather were purchased at The Tailored Hide in Neenah, Wis., a “naked leather” comprising “Vegtan” leather (leather tanned with vegetable matter) and a grained pigmented brown Nassa cowhide. Both had a smoother finished side and a coarser unfinished side and both had a nominal basis weight of 2 to 3 ounces, later measured at 808 g/m² (Nassa) and 813 g/m² (vegtan).

In testing, a pipette was used to apply droplets of the various solutions to give from 0.05 to 0.15 g drops on the unfinished side of the leather, and the time required to absorb into the leather was measured. The moment of full absorption was taken as them time when the reflection of overhead lights could no longer be seen on the moisture on the droplet surface, meaning there was no longer a smooth liquid surface to reflect the light (six distinct lights were distributed over the ceiling above the test area). This was done for at least 6 drops on various locations on each leather material. For a given material, the absorption time versus drop mass was plotted and fit to a linear curve, and the time for absorption for a droplet of 0.09 g was taken from the linear curve fit as the “effective absorption time.”

For rough side of the Vegtan material, the effective absorption times for fluids A, B, C, and D (due to limited space on the leather sample, E and F were not tested) were A: 29 s, B: 29 s, C: 27 s, and D: 22.5 s. For the Nassa leather sample, the results were A: 160 s, B: 148 s, C: 110 s, D: 97 s, E: 145 s, and F: 80 s. Some testing on the smooth side of the Vegtan leather was also conducted, but with different results, possibly due to an interaction from the chemical or other treatment on that side. This gave A: 76 s, B: 93 s, C: 83 s, and D: 92 s. Further testing with A and B gave consistent values. Why the finished side of the Vegtan exhibited such different behavior is unclear, but since the objective was to test the agents on the unfinished, porous side of the leather, those results suggest that while B was better that A in the Nassa leather and essentially the same in the Vegtan leather, there was a more significant decrease in absorption time with the panthenol present in sample C for both leathers. A small amount of propanediol also appeared to give a significant boost in the absorption rate. It may be that the combination of NAC with panthenol is particularly suited at enhancing skin or leather penetration of a Vitamin C serum, and pronanediol or ethoxydiglycol may also assist. Leather testing is, of course, not an ideal analog for skin absorption, but may still provide useful insight.

### REMARKS

When introducing elements of aspects of the invention or the embodiments thereof, the articles “a,” “an,” “the,” and “said” are intended to mean that there are one or more of the elements, and thus may include plural referents unless the context clearly dictates otherwise. The terms “comprising,” “including,” and “having” are intended to be inclusive and mean that there may be additional elements other than the listed elements.

Unless otherwise indicated, any individual ingredient or grouping of two or more individual ingredients in a list of ingredients for any particular function may be considered optional and, in fact, may be excluded from the overall composition, as desired, and if excluded, the product or composition may be “substantially free” of that ingredient. For example, in listing various NAC odor control agents, any one or more of those listed may be selected to be substantially absent in the composition. Thus, unless otherwise indicated, a list of potential ingredients should be understood as also providing support for embodiments that exclude those ingredients or are “substantially free” of them.

Unless otherwise specified, all patents and patent applications mentioned herein should be understood to be hereby incorporated by reference to the extent they are non-contradictory herewith. Further, all aspects of any invention may be, when not clearly improper, combined with any other aspect, such that any limitation in one claim or other aspect can be inherently available for combination with other aspects and limitations.

Having described aspects of the invention in detail, it will be apparent that modifications and variations are possible without departing from the scope of aspects of the invention as defined in the appended claims. As various changes could be made in the above compositions, products, and methods without departing from the scope of aspects of the invention, it is intended that all matter contained in the above description shall be interpreted as illustrative and not in a limiting sense.

While the foregoing description makes reference to particular illustrative embodiments, these examples should not be construed as limitations. The inventive system, methods, and products can be adapted for other uses or provided in other forms not explicitly listed above, and can be modified in numerous ways within the spirit of the present disclosure. Thus, the present invention is not limited to the disclosed embodiments, but is to be accorded the widest scope consistent with the claims below.

We claim:

1. A skin-care serum comprising from 0.2% to 6% N-acetyl cysteine, from 5% to 20% Vitamin C or derivative thereof, and from 0.2% to 3% panthenol or derivatives thereof, having a pH from 2.4 to 4.3, being substantially free of acrylamide compounds and having less than 15% lipids.

2. The serum of claim 1 further comprising an agent effective at reducing the odor of N-acetyl cysteine selected from phosphate salts, polyphosphate compounds, betaine compounds, sultaine compounds, and EDTA.

3. The serum of claim 2, comprising phosphate salts or polyphosphate compounds having a mass of at least 20% the mass of the N-acetyl cysteine.

4. The serum of claim 1 further comprising at least 0.01% cationic steroidal antimicrobials.

5. The serum of claim 1 having a pH from 2.4 to 3.5 and comprising less than 10% lipids.

6. The serum of claim 1 wherein the lipids comprise a plant-derived emulsifier.

7. A cosmetic or cleaning preparation comprising at least 0.5% N-acetyl cysteine, at least 1% of a diol or polyol, and an agent effective at reducing the odor of N-acetyl cysteine selected from phosphate salts, polyphosphate compounds, betaine compounds, sultaine compounds, and EDTA.

8. The preparation of claim 7, wherein the ratio of the mass of phosphate salts and polyphosphate compounds is at least 20% of the mass of N-acetyl cysteine.

9. The preparation of claim 7, further comprising at least 0.1% panthenol or a derivative thereof.

10. The preparation of claim 7 in the form of a solid stick for application to a surface, further comprising 10% to 80% of a hydrophobic base comprising lipids.

11. The preparation of claim 10 comprising from 1% to 20% water, 0.5% to 5% N-acetyl cysteine, 25% to 80% lipids, having an effective pH of 2.5 to 4.

12. The preparation of claim 11 further comprising at least 4% of a starch and from 1% to 8% of an alpha-carboxylic acid.

13. The preparation of claim 10, further comprising silicone material selected from at least one of a dimethicone or dimethicone derivative, a siloxane, and polymethylsilsequioxane, wherein the silicone material comprises from 10% to 35% of the preparation.

14. The preparation of claim 10 comprising less than 8% water and from 5% to 40% silicone material and from 15% to 70% lipids.

15. The preparation of claim 7 further comprising an acidic material selected from Vitamin C and derivatives thereof and alpha hydroxy acids, wherein the acidic material plus the N-acetyl cysteine comprises from 3% to 16% of the preparation, and wherein the preparation comprises at least 40% of a hydrophobic material selected from lipids and silicone materials, and comprises from 0.5% to 10% emulsifiers and from 1% to 25% of a solvent that is liquid at 25° C. selected from water, alcohols, diols, and polyols, wherein the acidic material is soluble in the solvent and the preparation is a solid at 25° C. having an effective pH from 2 to 4.5.

16. The preparation of claim 15, wherein the preparation is provided with indicia directing users to apply the material to skin to reduce body odor.

17. The preparation of claim 7 comprising less than 10% water and having an effective pH of 2.5 to 4.0.

18. A personal care product comprising 0.5% to 5% N-acetyl cysteine, at least 10% water, an odor control agent effective at reducing the odor of N-acetyl cysteine selected from phosphate salts, polyphosphate compounds, betaine compounds, sultaine compounds, and EDTA, wherein the mass ratio of the odor control agent relative to N-acetyl cysteine is at least 20%.

19. The personal care product of claim 18, further comprising 0.1 to 3% panthenol or derivatives thereof.

20. The personal care product of claim 18, wherein the product further comprises antifungal agents and is provided with packaging having indicia directing the user to apply to the personal care product to the toes for at least one of treatment or prevention of fungal infection.
